(19) United States
(12) Patent Application Publication
(10) Pub. No.: US 2008/0146536 A1
(43) Pub. Date: Jun. 19, 2008

(54) 2-Aminoimidazopyridines for treating neurodegenerative diseases

(75) Inventors: Andrew G. Cole, Robbinsville, NJ (US); Brian F. McGuinness, Plainsboro, NJ (US); Marc-Raleigh Brescia, Dayton, NJ (US); Yuefei Shao, Princeton, NJ (US); Guizhen Dong, Dayton, NJ (US); Ian Henderson, Hopewell, NJ (US)

Correspondence Address: HESLIN ROTHENBERG FARLEY & MESITI PC, 5 COLUMBIA CIRCLE, ALBANY, NY, 12203, US

(73) Assignee: Pharmacopeia, Inc., Cranbury, NJ (US)

(21) Appl. No.: 11/464,951

(22) Filed: Aug. 16, 2006

Related U.S. Application Data
(60) Provisional application No. 60/708,667, filed on Aug. 16, 2005.

(51) Int. Cl. A61K 31/397; C07D 221/04; C07D 295/00; A61K 31/496; A61K 31/55; A61K 31/553; A61P 25/00; C07D 267/10; C07D 223/02; A61K 31/5377; A61K 31/437

(52) U.S. Cl. 514/210.18; 546/118; 514/303; 544/127; 514/234.2; 544/362; 514/253.04; 540/597; 514/217.07; 540/544; 514/211.15

(57) ABSTRACT

The invention relates to 2-aminoimidazopyridine derivatives useful in treating disorders that are mediated by A₂ₐ receptor function, including neurodegenerative diseases including Parkinson's disease and inflammation. The compounds have general formula I:

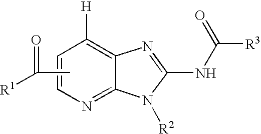

### CROSS REFERENCE TO RELATED APPLICATIONS

This application claims benefit from U.S. Provisional Application 60/708,667, filed Aug. 16, 2005, the entire contents of which are incorporated herein by reference.

### FIELD OF THE INVENTION

The invention relates to 2-aminoimidazopyridine derivatives useful in treating disorders that are mediated by adenosine receptor function, including neurodegenerative diseases and inflammation.

### BACKGROUND OF THE INVENTION

Adenosine is a modulator of multiple physiological functions, including cardiovascular, neurological, respiratory and renal functions. Adenosine mediates its effects through specific G-protein coupled membrane receptors A₁, A₂ₐ, A_{2b} and A₃.

Adenosine 2a (A₂ₐ) receptor antagonists useful in the treatment of Parkinson's disease have been disclosed in U.S. Pat. No. 6,875,772 and U.S. Pat. No. 6,787,541. Additionally, the application of A₂ₐ receptor antagonists in the treatment of restless leg syndrome is outlined in WO 2004019949. These disclosures are incorporated herein by reference as they relate to utility.

### SUMMARY OF THE INVENTION

In one aspect the present invention provides compounds according to formula I useful as adenosine 2a receptor antagonists:

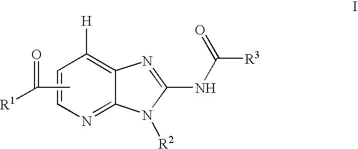

In these compounds
R¹ is selected from the group consisting of OR⁴, NR⁵R⁶, heterocyclyl and substituted heterocyclyl;
R² is selected from the group consisting of
   (a) C₁-C₂₀ hydrocarbon;
   (b) C₃-C₂₀ hydrocarbon in which
      (i) from one to three —CH₂— are replaced by —O—, —S(O)ₘ—, —NH— or —(C═(O)—, wherein m is 0, 1 or 2; or
      (ii) one

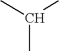

is replaced by

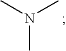

(c) heteroaryl and
(d) heteroarylalkyl;
R³ is selected from the group consisting of aryl, arylalkyl, heteroaryl, heteroarylalkyl, substituted aryl, substituted arylalkyl, substituted heteroaryl and substituted heteroarylalkyl;
R⁴ is selected from the group consisting of H, alkyl, aryl, arylalkyl, heteroaryl, heteroarylalkyl, substituted aryl substituted arylalkyl, substituted heteroaryl and substituted heteroarylalkyl;
R⁵ is selected from the group consisting of H, C₁-C₂₀ hydrocarbon, heterocyclyl, heterocyclylalkyl, substituted alkyl, oxaalkyl, substituted aryl, substituted arylalkyl, substituted heterocyclyl and substituted heterocyclylalkyl; and
R⁶ is selected from the group consisting of H, (C₁-C₆)alkyl, (C₁-C₆)alkoxy, aryl and substituted (C₁-C₆)alkyl.

In another aspect, the invention relates to pharmaceutical compositions comprising a therapeutically effective amount of at least one compound of general formula I or a pharmaceutically acceptable salt thereof, and a pharmaceutically acceptable carrier.

The compounds and pharmaceutical compositions described herein are useful in methods for preventing and treating a condition for which an antagonist of adenosine 2a receptor is indicated.

In a third aspect, the invention relates to a method for treating a disease by antagonizing a response mediated by adenosine 2a receptors. The method comprises bringing into contact with adenosine receptor at least one compound of general formula I or a pharmaceutically acceptable salt thereof.

In yet another aspect the present invention relates to a method of treating disease mediated by adenosine receptors (in addition to 2a) in a subject in need thereof comprising administering to the subject a therapeutically effective amount of at least one compound of general formula I or a pharmaceutically acceptable salt thereof.

The compounds of the present invention are useful in preventing and treating diseases and disorders mediated by adenosine 2a receptors, including neurological diseases and disorders.

The compounds of the present invention are useful in effecting neuroprotection and as such the present invention provides a method of neuroprotection in a subject in need thereof comprising administering to the subject a therapeutically effective amount of at least one compound of general formula I or a pharmaceutically acceptable salt thereof.

The compounds of the present invention are useful in treating movement disorders and dyskinesias and as such the present invention provides a method of neuroprotection in a subject in need thereof comprising administering to the subject a therapeutically effective amount of at least one compound of general formula I or a pharmaceutically acceptable salt thereof.

Other indications in which the adenosine antagonists are useful include central nervous system disorders, neurodegenerative diseases, and neurodevelopmental disorders.

In another aspect the present invention provides a method of treating a disorder associated with adenosine receptor function, including A₂ₐ receptors and one or more additional adenosine receptors, such as A₁, A_{2b} or A₃ receptors.

The compounds of the present invention are useful in stand alone treatments or in combination with one or more of (1) an agent useful in the treatment of Parkinson's disease, i.e. L-dopa, caffeine or other dopaminergic receptor agonist (2) an agent useful in the treatment of movement disorders, (3) an agent useful in the treatment of depression

### DETAILED DESCRIPTION OF THE INVENTION

Throughout this specification the substituents are defined when introduced and retain their definitions.

It has now been found that compounds of general formula I are potent adenosine 2a (A₂ₐ) receptor antagonists.

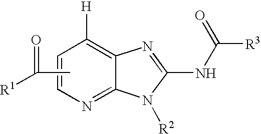

The compounds of formula I can be conveniently subdivided into three subgenera based on the value of R¹. When R¹ is NR⁵R⁶ a subgenus of amido 2-aminoimidazopyridines arises, having chemical formula II as shown below:

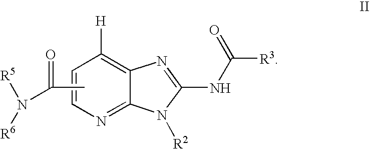

When R¹ is OR⁴ a subgenus of 2-aminoimiazopyridine esters arises, having chemical formula III as shown below:

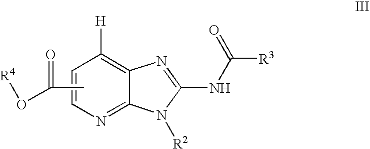

A third major subgenus IV is formed at the overlap of the two subgenera in which R¹ is NR⁵R⁶ and in which R¹ is heterocyclyl:

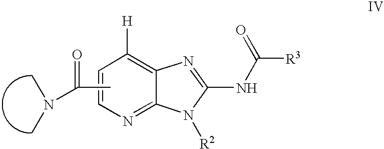

In this particular subgenus, R¹ is an N-attached heterocycle, such as piperidine, piperazine, pyrrolidine, morpholine, azepane, oxazepane, azetidine, indoline, isoindoline and tetrahydroisoquinoline, or a substituted N-attached heterocycle, such as N-benzylpiperazine, N-methylpiperazine, dimethylpiperidine, methoxypyrrolidine, dimethylaminopyrrolidine, pyrrolidinylpiperidine and trifluoromethylpyrrolidine. In certain embodiments the nitrogen-attached heterocycle may be described by the formuale:

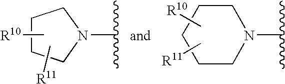

In these formulae, R¹⁰ and R¹¹ are independently chosen from H, (C₁-C₃)alkyl, halogen, halo(C₁-C₃)alkyl, hydroxy, hydroxy(C₁-C₃)alkyl, (C₁-C₃)oxaalkyl and (C₁-C₃)alkoxy, or taken together R¹⁰ and R¹¹ form a fused six-membered ring optionally substituted with (C₁-C₃)alkyl, halogen, halo(C₁-C₃)alkyl, hydroxy or (C₁-C₃)alkoxy.

In another embodiment, R¹ is optionally substituted aryl, such as phenyl, or optionally substituted heteroaryl, such as furan-3-yl, pyridin-3-yl, thiazol-2-yl, or isoxazol-5-yl.

A subgenus of I comprises compounds wherein R³ is heteroaryl, such as furanyl, thienyl, pyridinyl, indolyl and isoxazolyl; the heteroaryl may be optionally substituted. In another subgenus, R³ is selected from phenyl, phenylalkyl, substituted phenyl and substituted phenylalkyl, having chemical formula shown below:

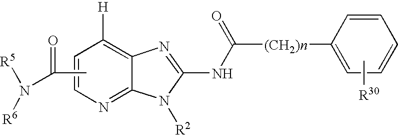

where n is 0 or an integer selected from 1-4; and
R³⁰ is selected from H, halogen (e.g. fluorine and chlorine), cyano, nitro, formyl, alkoxy (e.g. methoxy), alkyl, haloalkyl (e.g. trifluoromethyl), alkynyl (e.g. acetylenyl) and heteroaryl (e.g. pyrazolyl).

In some embodiments n is 0 and R³⁰ is cyano or fluoro.

In some embodiments R² is chosen from methoxyphenyl, 1-acetylpiperidinyl, 1-acetylpyrrolidinyl, 1-acetylazetidinyl tetrahydrofuranyl, tetrahydrofuranylmethyl, tetrahydropyranyl, pyridinylmethyl, (imidazolyl)ethyl, (methylimidazolyl)ethyl, methylpiperidinyl, pyrimidinylmethyl, (acetylamino)(C₁-C₆)alkyl, hydroxy(C₁-C₆)alkyl, methylthio(C₁-C₆)alkyl, α-pyridinylethyl, (oxopyrrolidinyl)propyl, cyclopropyl, [(C₁-C₆)alkoxycarbonyl](C₁-C₆)alkyl, (methylamino)(C₁-C₆)alkyl, α(methoxyphenyl)ethyl and (dimethoxyphenyl)methyl. In other embodiments, R² is alkoxyalkyl. In certain embodiments R² is methoxypropyl.

In some embodiments R³ is selected from phenyl, phenylalkyl, substituted phenyl and substituted phenylalkyl; and R² is methoxypropyl, having chemical formulae as shown below:

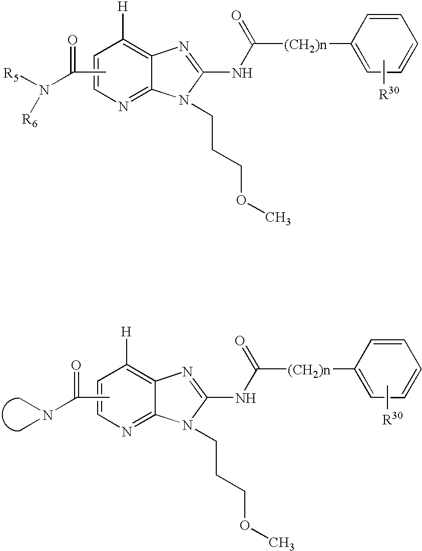

In specific embodiments, R³ is cyanophenyl and R² is methoxypropyl.

In some embodiments R⁶ is H.

In some embodiments R⁵ is selected from C₁-C₂₀ hydrocarbon, substituted aryl and substituted arylalkyl. In certain embodiments R⁵ is C₁-C₂₀ hydrocarbon. In specific embodiments R⁵ is selected from benzyl and substituted benzyl. In some embodiments R⁶ is hydrogen or (C₁-C₃)alkyl and R⁵ is —(CH₂)ₙ-cyc. In these compounds n is 1 to 4 and cyc is carbocyclyl or heterocyclyl (e.g. phenyl, pyridinyl, imidazolyl and pyrrolidinyl), which may be optionally substituted with from one to three halogen, (C₁-C₃)alkyl, hydroxy or (C₁-C₃)alkoxy.

Subgenus III comprises compounds wherein R³ is selected from phenyl, phenylalkyl, substituted phenyl and substituted phenylalkyl, having chemical formula shown below:

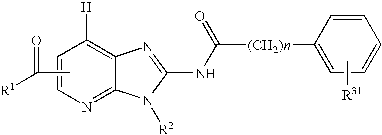

where n is 0 or an integer selected from 1-4; and
R³¹ is selected from H, halogen, cyano, nitro, alkoxy, alkyl and haloalkyl (e.g. trifluoromethyl).

In some embodiments R² is alkoxyalkyl. In certain embodiments R² is methoxypropyl.

In some embodiments R³ is selected from phenyl, phenylalkyl, substituted phenyl and substituted phenylalkyl; and R² is methoxypropyl, having chemical formulae as shown below:

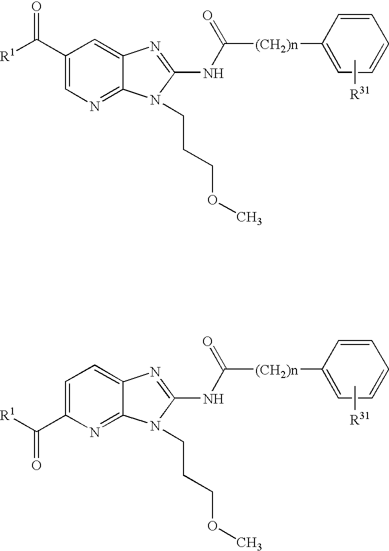

These two structures depict the two possible isomers that are encompassed by the earlier generic formula in which the RICO residue is shown indeterminately at 5 or 6, but not 7.

In some embodiments, n is 0. In some embodiments R³¹ is cyano. In some embodiments R⁴ is H. In other embodiments R⁴ is alkyl.

In certain embodiments, R² is methoxypropyl and R³ is cyanophenyl.

In a second aspect the present invention provides a pharmaceutical composition comprising a pharmaceutically acceptable carrier and a therapeutically effective amount of at least one compound according to formula I.

In a third aspect the present invention provides a method of treating a disorder, which is mediated by adenosine 2a (A₂ₐ) receptor function, which comprises administering to a subject in need of such treatment a therapeutically effective amount of a compound of formula I.

All though the compounds of the invention are selective A₂ₐ antagonists, some of them may exhibit sufficient residual affinity for other classes of adenosine receptors to be useful to treat conditions associated with additional adenosine receptors. As a result, the present invention also provides a method of treating a disorder associated with the A₂ₐ receptor and one or more of A₁, A_{2b} or A₃ receptors.

All of the compounds falling within the foregoing parent genera and their subgenera are useful as adenosine receptor antagonists.

For convenience and clarity certain terms employed in the specification, examples and claims are described below.

Alkyl is intended to include linear, branched, or cyclic hydrocarbon structures and combinations thereof. Lower alkyl refers to alkyl groups of from 1 to 6 carbon atoms. Examples of lower alkyl groups include methyl, ethyl, propyl, isopropyl, butyl, s- and t-butyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Examples include ethylaminoethyl and methylthiopropyl.

C₁ to C₂₀ hydrocarbon includes alkyl, cycloalkyl, alkenyl, alkynyl, aryl, arylalkyl and combinations thereof. Examples include benzyl, phenethyl, cyclohexylmethyl, camphoryl, adamantyl and naphthylethyl. The application refers to C₃-C₂₀ hydrocarbon in which from one to three —CH₂— are replaced by —O—, —S(O)ₘ—, —NH— or —(C═O)—. Examples would be:

| | (C₃-C₂₀)hydrocarbon |
|---|---|
| (C₃-C₂₀)hydrocarbon | with CH₂ replaced with —O— |
| 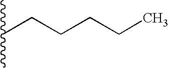 | 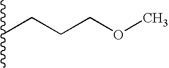 |
| 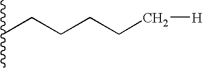 | 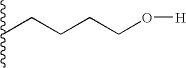 |
| 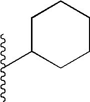 | 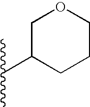 |
| 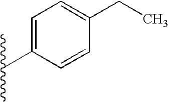 | 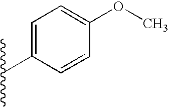 |

The other replacements follow the same pattern. Thus 3-(dimethylamino)propyl can be envisioned as 4-methylpentyl wherein one

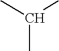

is replaced by

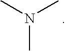

Alkoxy or alkoxyl refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons.

Alkoxyalkyl refers to ether groups of from 3 to 8 atoms of a straight, branched, cyclic configuration and combinations thereof attached to the parent structure through an alkyl. Examples include methoxymethyl, methoxyethyl, ethoxypropyl, and the like.

Alkoxyaryl refers to alkoxy substituents attached to an aryl, wherein the aryl is attached to the parent structure.

Acyl refers to groups of from 1 to 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. The aromatic 6- to 14-membered carbocyclic rings include, e.g., benzene and naphthalene, and according to the invention benzoxalane and residues in which one or more rings are aromatic, but not all need be. The 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

Arylalkyl refers to a substituent in which an aryl residue is attached to the parent structure through alkyl. Examples are benzyl, phenethyl and the like. Heteroarylalkyl refers to a substituent in which a heteroaryl residue is attached to the parent structure through alkyl. Examples include, e.g., pyridinylmethyl, pyrimidinylethyl and the like.

Heterocycle means a cycloalkyl or aryl residue in which from one to three carbons is replaced by a heteroatom selected from the group consisting of N, O and S. The nitrogen and sulfur heteroatoms may optionally be oxidized, and the nitrogen heteroatom may optionally be quaternized. Examples of heterocycles include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. It is to be noted that heteroaryl is a subset of heterocycle in which the heterocycle is aromatic. According to convention, the suffix “yl” indicates the moiety in question appearing as a residue on a parent structure. Thus, for example, heterocyclyl means a heterocycle appearing as a substituent rather than a parent. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

An oxygen heterocycle is a heterocycle containing at least one oxygen in the ring; it may contain additional oxygens, as well as other heteroatoms. A sulphur heterocycle is a heterocycle containing at least one sulphur in the ring; it may contain additional sulphurs, as well as other heteroatoms. Oxygen heteroaryl is a subset of oxygen heterocycle; examples include furan and oxazole. Sulphur heteroaryl is a subset of sulphur heterocycle; examples include thiophene and thiazine. A nitrogen heterocycle is a heterocycle containing at least one nitrogen in the ring; it may contain additional nitrogens, as well as other heteroatoms. Examples include piperidine, piperazine, morpholine, pyrrolidine and thiomorpholine. Nitrogen heteroaryl is a subset of nitrogen heterocycle; examples include pyridine, pyrrole and thiazole.

Substituted alkyl, aryl, cycloalkyl, heterocyclyl etc. refer to alkyl aryl, cycloalkyl, or heterocyclyl wherein up to three H atoms in each residue are replaced with halogen, haloalkyl, alkyl hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heterocyclyl (including heteroaryl), phenoxy, benzyloxy, or heteroaryloxy. When, in the above list, the substituent is phenyl, phenoxy or heteroaryl, the phenyl, phenoxy or heteroaryl may itself be substituted with halogen, haloalkyl, alkyl hydroxy or loweralkoxy.

The terms “halogen” and “halo” refer to fluorine, chlorine, bromine or iodine.

Some of the compounds described herein may contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, as well as, their racemic and optically pure forms. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration; thus a carbon-carbon double bond depicted arbitrarily herein as trans may be Z, E or a mixture of the two in any proportion.

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but denoting racemic character, and wedge outlines and dotted or broken lines denote enantiomerically pure compounds of indeterminate absolute configuration.

It will be recognized that the compounds of this invention can exist in radiolabeled form, i.e., the compounds may contain one or more atoms containing an atomic mass or mass number different from the atomic mass or mass number usually found in nature. Radioisotopes of hydrogen, carbon, phosphorous, fluorine, chlorine and iodine include ³H, ¹⁴C, ³⁵S, ¹⁸F, ³⁶Cl and ¹²⁵I, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Radiolabeled compounds of this invention can generally be prepared by methods well known to those skilled in the art. Conveniently, such radiolabeled compounds can be prepared by carrying out the procedures disclosed in the Examples by substituting a readily available radiolabeled reagent for a non-radiolabeled reagent. Because of the high affinity for the A2a receptor, radiolabeled compounds of the invention are useful for A2a receptor assays.

Terminology related to “protecting”, “deprotecting” and “protected” functionalities occurs throughout this application. Such terminology is well understood by persons of skill in the art and is used in the context of processes that involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry, such as Protective Groups in Organic Synthesis by T. W. Greene [John Wiley & Sons, New York, 1991], which is incorporated herein by reference.

A comprehensive list of abbreviations utilized by organic chemists appears in the first issue of each volume of the Journal of Organic Chemistry. The list, which is typically presented in a table entitled “Standard List of Abbreviations”, is incorporated herein by reference.

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here. The starting materials, for example in the case of suitably substituted imidazopyridine ring compounds, are either commercially available, synthesized as described in the examples or may be obtained by the methods well known to persons of skill in the art.

The present invention further provides pharmaceutical compositions comprising as active agents, the compounds described herein.

As used herein a “pharmaceutical composition” refers to a preparation of one or more of the compounds described herein, or physiologically acceptable salts or solvents thereof, with other chemical components such as physiologically suitable carriers and excipients.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

Compounds that antagonize the adenosine receptor can be formulated as pharmaceutical compositions and administered to a mammalian subject, such as a human patient in a variety of forms adapted to the chosen route of administration, i.e., orally or parenterally, by intravenous, intramuscular, topical, transdermal or subcutaneous routes.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar or alginic acid or a salt thereof such as sodium alginate.

In addition, enteric coating may be useful as it is may be desirable to prevent exposure of the compounds of the invention to the gastric environment.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For injection, the compounds of the invention may be formulated in aqueous solutions, preferably in physiologically compatible buffers such as Hank's or Ringer's solution or physiological saline buffer. For transmucosal and transdermal administration, penetrants appropriate to the barrier to be permeated may be used in the composition. Such penetrants, including for example DMSO or polyethylene glycol, are known in the art.

For administration by inhalation, the compounds for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds, to allow for the preparation of highly concentrated solutions.

The compounds of the present invention may also be formulated in rectal compositions such as suppositories or retention enemas, using, e.g., conventional suppository bases such as cocoa butter or other glycerides.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on many factors including the subject being treated, the severity of the affliction, the manner of administration, the judgment of the prescribing physician. The compounds of the invention may be administered orally or via injection at a dose from 0.001 to 2500 mg/kg per day. The dose range for adult humans is generally from 0.005 mg to 10 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg. The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity.

As used herein, and as would be understood by the person of skill in the art, the recitation of “a compound” is intended to include salts, solvates and inclusion complexes of that compound. The term “solvate” refers to a compound of Formula I in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions. Inclusion complexes are described in Remington: The Science and Practice of Pharmacy 19th Ed. (1995) volume 1, page 176-177, which is incorporated herein by reference. The most commonly employed inclusion complexes are those with cyclodextrins, and all cyclodextrin complexes, natural and synthetic, are specifically encompassed within the claims.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine.

The term “preventing” as used herein refers to administering a medicament beforehand to forestall or obtund an attack. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended herein.

It should be understood that in addition to the ingredients particularly mentioned above, the formulations of this invention may include other agents conventional in the art having regard to the type of formulation in question, for example those suitable for oral administration may include flavoring agents.

The compositions may be presented in a packaging device or dispenser, which may contain one or more unit dosage forms containing the active ingredient. Examples of a packaging device include metal or plastic foil, such as a blister pack and a nebulizer for inhalation. The packaging device or dispenser may be accompanied by instructions for administration. Compositions comprising a compound of the present invention formulated in a compatible pharmaceutical carrier may also be placed in an appropriate container and labeled for treatment of an indicated condition.

The compounds and compositions of the present invention may be used or administered in combination with additional agents useful in treating neurodegenerative disorders, movement disorders and the like. Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within any number of hours of each other or within any number or days or weeks of each other. In some cases even longer intervals are possible.

While in many cases it is desirable that the two or more agents used in a combination therapy be present in within the patient's body at the same time, this need not be so. Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

As antagonists of A₂ₐ receptors, the compounds of formula I have utility in treating and preventing inter alia neurodegenerative disorders and depression. The compounds and compositions can be used advantageously in combination with other agents useful in treating neurodegenerative disorders and depression. For example, a compound or compounds of formula I may be used in preparing a composition further comprising L-dopa and or caffeine for utility in the treatment of Parkinson's and related diseases.

The compounds of the present invention are useful in inhibiting the activity of A₂ₐ receptors or in inhibiting A₂ₐ receptor-mediated activity and are useful in treating complications arising therefrom.

According to the present invention, the A₂ₐ receptor antagonists may be administered prophylactically, i.e., prior to onset of acute allergic reaction, or they may be administered after onset of the reaction, or at both times.

A₂ₐ antagonists have been shown to produce an increase in locomotor activity, a decrease of neuroleptic-induced catalepsy, decrease of MPTP-induced hypomotility, reversal of cocaine withdrawal-induced anhedonia and several indications of neuroprotection in response to brain injury. These observation support therapeutic indications of A₂ₐ antagonists for inter alia Parkinson's disease (PD) and cocaine abuse, and neurodegenerative disorders such as Alzheimer's disease.

A₂ₐ antagonists, such as SCH 58261 and KW-6002, are particularly compelling for the treatment of PD since they not only enhance locomotor activity in animal models as a stand-alone treatment, but they potentiate the activity of L-dopa so that levels of L-dopa with reduced propensity to elicit dyskenesias can be given (Chen, Drug News Perspect. 2003, 16, 597; Morelli et al, Drug Dev. Res. 2001, 52, 387; Bara-Jimenez et al, Neurology 2003, 61, 293). Furthermore, the efficacy of A₂ₐ antagonists does not diminish upon repeated exposure, as seen for L-dopa (Halldner et al, Eur. J. Pharmacol. 2000, 406, 345). A distinct advantage of A₂ₐ antagonists over L-dopa is the propensity for neuroprotection (Morelli et al, Neurotox. Res. 2001, 3, 545).

The adenosine receptor antagonists of the present invention are useful in effecting neuroprotection and in treating central nervous system and peripheral nervous system diseases, neurodegenerative diseases, cardiovascular diseases, cognitive disorders, CNS injury, renal ischemia; acute and chronic pain; affective disorders; cognitive disorders; central nervous system injury; cerebral ischemia; myocardial ischemia; muscle ischemia; sleep disorders; eye disorders and diabetic neuropathy.

In some embodiments the CNS and PNS disorders are movement disorders. A movement disorder may be a disorder of the basal ganglia which results in dyskinesias. Non-limitative disorders include Huntington's disease, multiple system atrophy, progressive supernuclear palsy, essential tremor, myoclonus, corticobasal degeneration, Wilson's disease, progressive pallidal atrophy, Dopa-responsive dystoma-Parkinsonism, spasticity, Alzheimer's disease and Parkinson's disease. Parkinson's disease further includes early-onset Parkinson's disease, drug-induced Parkinsonism, post-encephalitic Parkinsonism, Parkinsonism induced by poisoning and post-traumatic Parkinson's disease.

The compounds of the present invention have utility as neuroprotectants and may be useful in preventing or treating traumatic brain injury (TBI) and for the attenuation of cognitive impairment in coronary artery bypass graft (CABG) patients. As such the compounds and compositions may be administered to a subject at risk of neural ischemia.

The following examples will further describe the invention, and are used for the purposes of illustration only, and should not be considered as limiting the invention being disclosed.

### EXAMPLES

### Examples

Abbreviations: The following abbreviations and terms have the indicated meaning throughout, unless otherwise stated:

Ac—acetyl
AcOH—Acetic acid
Boc—tert-butoxycarbonyl
Boc₂O—tert-butoxycarbonic anhydride
Bu—butyl
C—carbon
c—cyclo
CDCl₃—Deuterated chloroform
CD₃OD—Deuterated methanol
δ—NMR chemical shift referenced to tetramethylsilane
DCE—1,2-dichloroethane
DCM—dichloromethane=methylene chloride=CH₂Cl₂
DIC—Diisopropyl carbodiimide

### DIPEA—Diisopropylethylamine

DMAP—4-Dimethylamino pyridine

### DMF—N,N-dimethylformamide

DMSO—Dimethyl sulfoxide
EDC—N-(3-Dimethylaminopropyl)ethylcarbodiimide hydrochloride salt

### Et—Ethyl

EtOAc—Ethyl acetate
ESI—Electrospray ionization

### Et₃N —Triethylamine

### Et₃SiH —Triethylsilane

### ¹H NMR—Proton Nuclear Magnetic Resonance

h—hours
Hexanes —HPLC grade isomeric hexanes
HOBt—hydroxybenzotriazole
i—iso

### LCMS—Liquid Chromatography Mass Spectroscopy

m—meta
Me—methyl
MeOH—methanol=CH₃OH
min—minutes
n—normal
N—nitrogen

### NMR—Nuclear Magnetic Resonance

NaBH₄—sodium borohydride
NaCNBH₃—sodium cyano borohydride
Na(OAc)₃BH—sodium triacetoxy borohydride
o—ortho
p—para

### Ph—Phenyl

r.t. —room temperature
sat. —saturated
s—secondary
t—tertiary
TFA—trifluoro acetic acid
THF—tetrahydrofuran

### Synthesis of 2-aminoimidazopyridines

Compounds of type I can be synthesized by means of conventional organic synthesis employing solid-phase and solution-phase chemistries, executable by those skilled in the art. The illustration of examples, but not the limitation, of the synthesis of compounds of type I is detailed below:

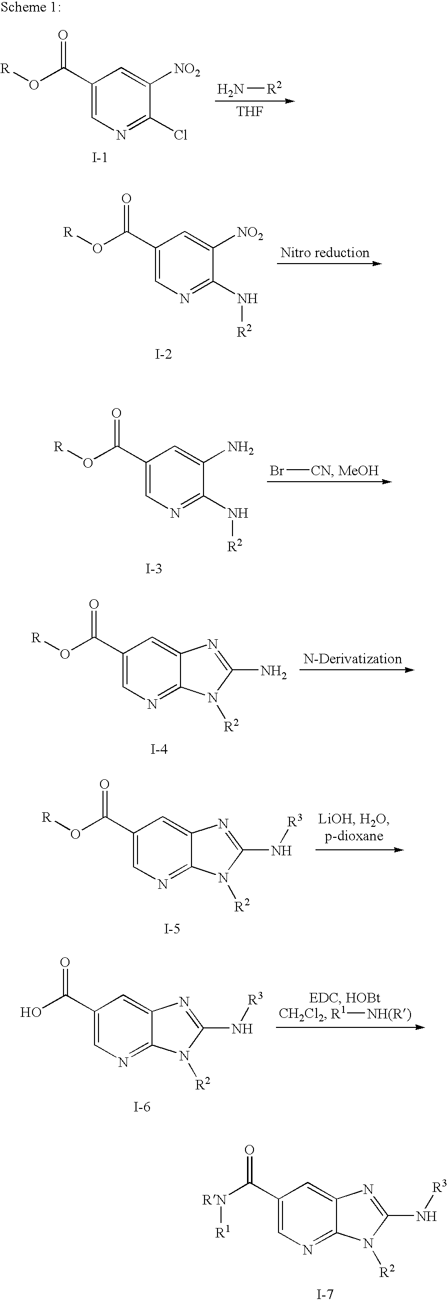

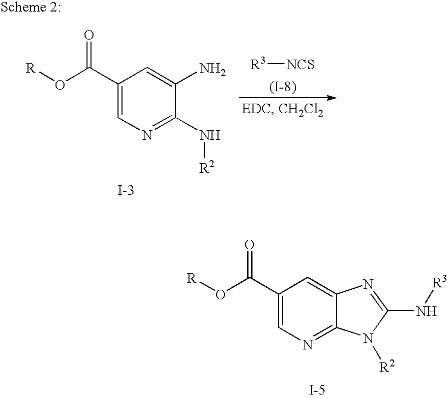

### General Procedures

Compounds of formula I (type I-7) were synthesized in a multi-step synthesis from commercially available 6-hydroxy-nicotinic acid. Nitration of 6-hydroxy-nicotinic acid and subsequent conversion to ethyl 6-chloro-5-nitronicotinate (I-1) is detailed by Berrie et al, (J. Chem. Soc. 2590, 1951). The synthesis of methyl 6-chloro-5-nitronicotinate can be readily accomplished using a similar procedure. Arylation of a primary amine with I-1 followed by nitro reduction allows amino imidazopyridine formation with cyanogen bromide to provide I-4 (Scheme 1). Functionalization of the amino group followed by ester hydrolysis with lithium hydroxide allows amide formation with an amine and EDC to provide compounds of formula I (type I-7). Alternatively, imidazopyridine formation to generate I-5 can be achieved directly from I-3 via reaction with functionalized isothiocyanates (Scheme 2). Functionalized isothiocyanates are either obtained from commercial sources or synthesized from the corresponding acid chloride. Analogous compounds of formula I can be synthesized using similar experimental procedures.

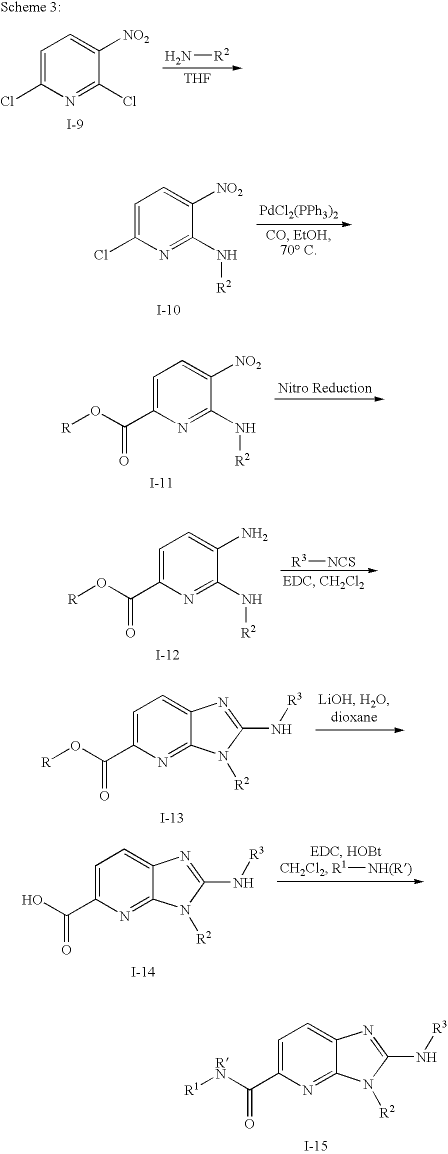

Compounds of formula I (type I-15) were synthesized in a multi-step synthesis from commercially available 2,6-dichloro-3-nitropyridine (Scheme 3). Arylation of a primary amine with 2,6-dichloro-3-nitropyridine (I-9) is followed by palladium catalyzed ester formation to generate I-11 (Scheme 3). Further synthetic manipulation is conducted following a similar sequence of reactions to those detailed previously. Nitro reduction to generate I-12 is followed by reaction with a functionalized isothiocyanate (I-8) to provide I-13. Ester hydrolysis followed by amide formation provides compounds of formula I (type I-15). Analogous compounds of formula I can be synthesized using similar experimental procedures.

### Intermediate 2 (I-2) Procedure A

### Ethyl 6-(3-methoxypropylamino)-5-nitronicotinate

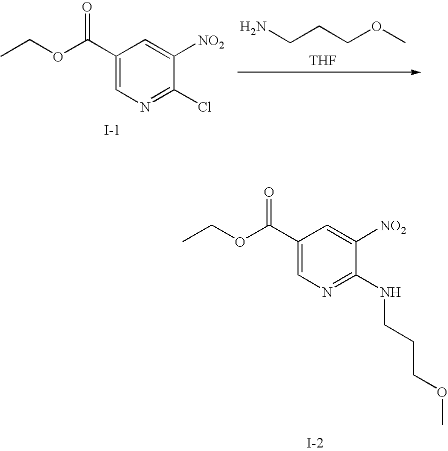

To a solution of 0.5 g (2.17 mmol, 1.0 eq.) of ethyl 6-chloro-5-nitronicotinate (I-1) in 15 mL of THF at 0° C. was added 1.5 mL (14.7 mmol, 6.7 eq.) of 3-methoxypropyl amine and the mixture stirred at room temperature for 24 h. The solvent was removed in vacuo, and the residue was purified by flash chromatography (30%-50% EtOAc/hexanes) to provide 0.44 g (1.55 mmol, 71%) of ethyl 6-(3-methoxypropylamino)-5-nitronicotinate (I-2) as a yellow solid. (δ_{H}, 300 MHz, CDCl₃) 1.38 (t, 3H), 1.95 (m, 2H), 3.40 (s, 3H), 3.55 (t, 2H), 3.80 (q, 2H), 4.36 (q, 2H), 8.86 (m, 2H); m/z (ESI) found 284.2 [M+H]⁺.

### Intermediate 3 (I-3) Procedure B

### Ethyl-amino-6-(3-methoxypropylamino)nicotinate

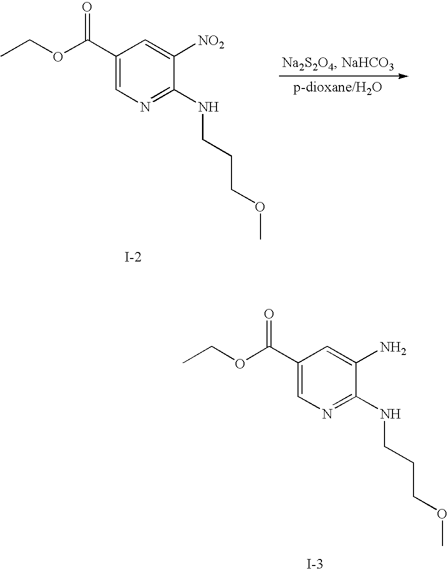

To a solution of 0.35 g (1.24 mmol, 1.0 eq.) of ethyl 6-(3-methoxypropylamino)-5-nitronicotinate (I-2) in 10 mL of p-dioxane was added a solution of 1.2 g (tech. grade ˜85%, ˜5.8 mmol, ˜4.6 eq.) of sodium hydrosulfite and 0.72 g (8.57 mmol, 6.9 eq.) of NaHCO₃ in 10 mL of water and the mixture stirred at room temperature for 1 h. The mixture was diluted with 50 mL of EtOAc and 30 mL of sat. NaHCO₃ (aq) and the layers separated. The organic phase was washed with 30 mL of sat. brine, dried (Na₂SO₄) and the solvent removed in vacuo to provide 0.20 g (0.79 mmol, 64%) of ethyl 5-amino-6-(3-methoxypropylamino)nicotinate (I-3). m/z (ESI) found 254.1 [M+H]⁺.

### Intermediate 5 (I-5) Procedure C

### Ethyl 2-amino-3-(3-methoxypropyl)-3H-imidazo[4,5-b]pyridine-6-carboxylate

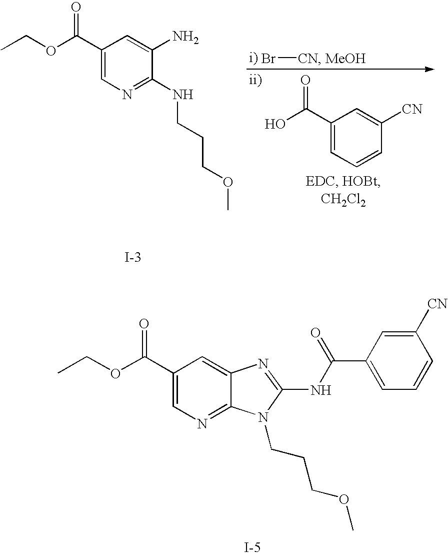

To a solution of 0.20 g (0.79 mmol, 1.0 eq.) of ethyl 5-amino-6-(3-methoxypropylamino)nicotinate (I-3) in 5 mL of MeOH was added 0.20 g (1.89 mmol, 2.4 eq.) of cyanogen bromide and the mixture stirred at room temperature for 2 h. The solvent was removed in vacuo and the residue dried under high vacuum. The crude aminoimidazopyridine was dissolved in 5 mL of CH₂Cl₂ and added to a suspension of 0.2 g (1.4 mmol, 1.8 eq.) of 3-cyanobenzoic acid, 0.2 g (1.31 mmol, 1.7 eq.) of HOBt monohydrate and 0.2 g (1.04 mmol, 1.3 eq.) of EDC in 5 mL of CH₂Cl₂ and the mixture stirred at room temperature for 3 h. The mixture was diluted with 20 mL of CH₂Cl₂ and washed with 10 mL of sat. Na₂CO₃ (aq). The organic phase was dried (Na₂SO₄) and the solvent removed in vacuo. The residue was purified by flash chromatography (20-60% EtOAc/hexanes) to provide 0.18 g (0.44 mmol, 56%) of ethyl 2-amino-3-(3-methoxypropyl)-3H-imidazo[4,5-b]pyridine-6-carboxylate (I-5) as a white solid. (δ_{H}, 300 MHz, CDCl₃) 1.43 (t, 3H), 2.22 (m, 2H), 3.30 (s, 3H), 3.50 (t, 2H), 4.45 (q, 2H), 4.52 (t, 2H), 7.59 (m, 1H), 7.80 (dd, 1H), 8.18 (d, 1H), 8.56 (dd, 1H), 8.68 (s, 1H), 9.00 (d, 1H); m/z (ESI), found 408 [M+H].

### Intermediate 6 (16) Procedure D

### 2-(3-cyanobenzamido)-3-(3-methoxypropyl)-3H-imidazo[4,5-b]pyridine-6-carboxylic acid

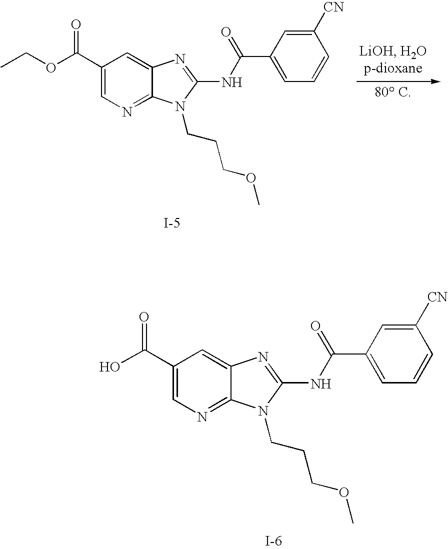

To a suspension of 0.15 g (0.37 mmol, 1.0 eq.) of ethyl 2-amino-3-(3-methoxypropyl)-3H-imidazo[4,5-b]pyridine-6-carboxylate (I-5) in 10 mL of p-dioxane was added a solution of 0.10 g (2.3 mmol, 6.4 eq.) of lithium hydroxide monohydrate in 5 mL of water and the mixture stirred at 80° C. for 30 min. The mixture was allowed to cool to room temperature and diluted with 50 mL of EtOAc and 10 mL of water. The mixture was acidified with 30 mL of 1 M HCl and the layers separated. The aqueous phase was extracted with 2×20 mL of EtOAc and the combined organic extracts dried (Na₂SO₄). The solvent was removed in vacuo to provide 0.12 g (0.32 mmol, 86%) of 2-(3-cyanobenzamido)-3-(3-methoxypropyl)-3H-imidazo[4,5-b]pyridine-6-carboxylic acid (I-6) as a white solid. (δ_{H}, 300 MHz, d⁶DMSO) 2.00 (m, 2H), 3.08 (s, 3H), 3.35 (t, 2H), 4.28 (t, 2H), 7.60 (m, 1H), 7.90 (d, 1H), 8.10 (d, 1H), 8.43 (d, 1H), 8.50 (s, 1H), 8.71 (d, 1H), 12.8 (bs, 1H), 13.1 (bs, 1H); m/z (ESI) found 380.3 [M+H]⁺.

### Intermediate 7 (I-7) Procedure E

### N-Butyl-2-(3-cyanobenzamido)-3-(3-methoxypropyl)-3H-imidazo[4,5-b]pyridine-6-carboxamide

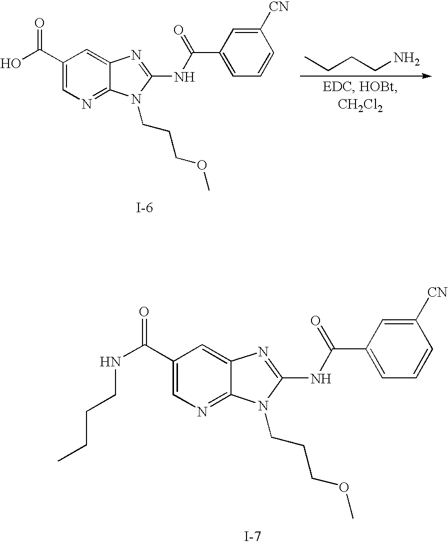

To a suspension of 15 mg (40 μmol, 1.0 eq.) of 2-(3-cyanobenzamido)-3-(3-methoxypropyl)-3H-imidazo[4,5-b]pyridine-6-carboxylic acid (I-6), 15 mg (0.1 mmol, 2.5 eq.) of HOBt monohydrate and 15 mg (80 umol, 2.0 eq.) of EDC in 1.5 mL of CH₂Cl₂ was added 30 uL (0.3 mmol, 7.6 eq.) of n-butyl amine and the mixture stirred at room temperature for 16 h. The mixture was diluted with 1 mL of CH₂Cl₂ and 1 mL of sat. NaHCO₃ (aq) and the layers separated. The organic phase was dried (Na₂SO₄) and the solvent removed in vacuo. The residue was purified by flash chromatography (40-80% EtOAc/hexanes) to provide 12 mg (28 μmol, 70%) of N-butyl-2-(3-cyanobenzamido)-3-(3-methoxypropyl)-3H-imidazo[4,5-b]pyridine-6-carboxamide (I-7) as a white solid. (δ_{H}, 300 MHz, CDCl₃) 0.98 (t, 3H), 1.45 (m, 2H), 1.65 (m, 2H), 2.23 (m, 2H), 3.40 (s, 3H), 3.52 (m, 4H), 4.51 (t, 2H), 6.35 (bt, 1H), 7.59 (t, 1H), 7.80 (m, 1H), 8.03 (d, 1H), 8.55 (m, 1H), 8.64 (d, 1H), 8.68 (m, 1H); nm/z (ESI) found 435.2 [M+H]⁺.

### Intermediate 8 (I-8) Procedure F

### 3-Cyanobenzoyl isothiocyanate

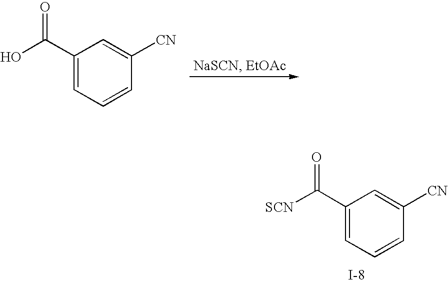

To a solution of 4.0 g (24 mmol. 1.0 eq.) of 3-cyanobenzoyl chloride in 100 mL of ethyl acetate was added 4.9 g (60 mmol, 2.5 eq.) of sodium thiocyanate and the mixture was stirred at room temperature for 16 h. The suspension was then diluted with 100 mL of hexanes and filtered through a Celite® plug. The filtrate was concentrated in vacuo and the residue purified by flash chromatography (50% EtOAc/hexanes) to provide 3.5 g (18.6 mmol 78%) of 3-cyanobenzoyl isothiocyanate (I-8) as pale a yellow solid.

### Intermediate 5 (I-5) Procedure G

### Methyl 2-(3-cyanobenzamido)-3-(3-methoxypropyl)-3H-imidazo[4,5-b]pyridine-6-carboxylate

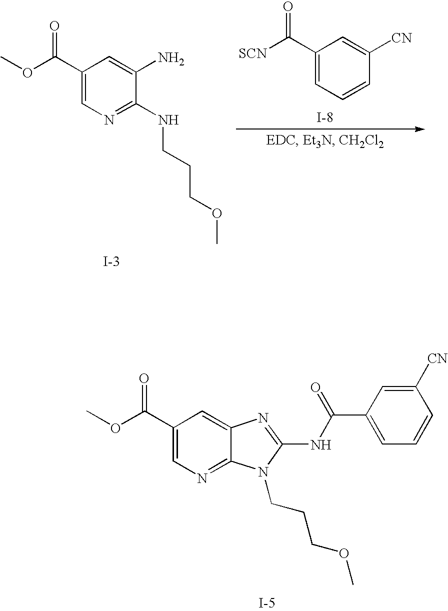

To a solution of 1.0 g (4.2 mmol, 1.0 eq.) of methyl 5-amino-6-(3-methoxypropylamino)nicotinate (I-3) in 20 mL of anhydrous CH₂Cl₂ was added 1.9 mL (13.7 mmol 3.0 eq.) of triethylamine followed by 0.78 g (4.2 mmol 1.0 eq.) of 3-cyanobenzoyl isothiocyanate (I-8) and 1.6 g (8.3 mmol 2.0 eq.) of EDC. The mixture was stirred at room temperature for 16 h. The reaction mixture was diluted with 20 mL of water the layers separated. The aqueous phase was extracted twice with CH₂Cl₂ and the combined organic extracts were washed with sat. brine, dried (Na₂SO₄) and the solvent removed in vacuo to yield the crude product. The crude product was washed with methanol to yield 0.66 g (1.7 mmol 40%) of methyl 2-(3-cyanobenzamido)-3-(3-methoxypropyl)-3H-imidazo[4,5-b]pyridine-6-carboxylate (I-5).

### Intermediate 10 (I-10) Procedure H

### 6-Chloro-N-(3-methoxypropyl)-3-nitropyridin-2-amine

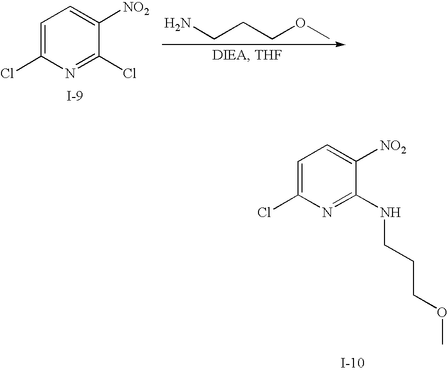

To a solution of 5 g (24 mmol, 1 eq.) of 2,6-dichloro-3-nitropyridine (I-9) in 95 mL of CH₂Cl₂ was added a solution of 2.3 mL (24 mmol, 1 eq.) of 3-methoxypropylamine in 5 mL of CH₂Cl₂ followed by 4.6 mL (36 mmol, 1.5 eq.) of diisopropylethylamine The resulting reaction mixture was stirred at room temperature for 16 h and then concentrated in vacuo. The residue was purified by flash chromatography (CH₂Cl₂) to afford 4.9 g (20 mmol, 83%) of 6-chloro-N-(3-methoxypropyl)-3-nitropyridin-2-amine (I-10) as a yellow solid; m/z (ESI) found 246, [M+H]⁺.

### Intermediate 11 (I-11) Procedure I

### Ethyl 6-(3-methoxypropylamino)-5-nitropicolinate

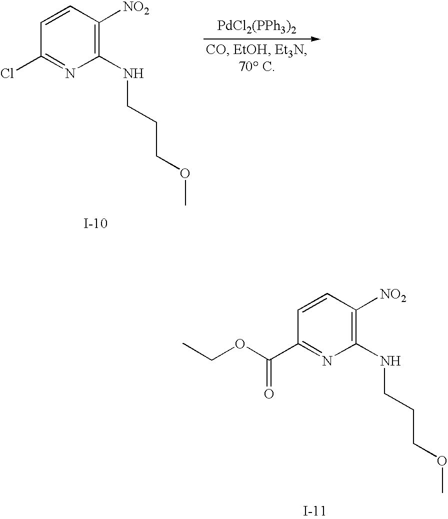

To a solution of 4.9 g (20 mmol, 1 eq.) of 6-chloro-N-(3-methoxypropyl)-3-nitropyridin-2-amine (I-10) in 90 mL of 2:1 v/v EtOH:Et₃N was added 1.4 g (2 mmol, 0.1 eq.) of PdCl₂(PPh₃)₂. Carbon monoxide gas was bubbled through the solution for 10 min and the mixture was then heated to 70° C. for 4 h with continuous CO bubbling. The reaction mixture was cooled to room temperature and then heated again at 70° C. for 16 h under carbon monoxide atmosphere. Celite® was added to the black reaction mixture and the volatiles were removed in vacuo. Purification of the residue on Celite® by flash chromatography (hexanes to 20% EtOAc/hexanes) afforded 2.1 g (7.4 mmol, 37%) of ethyl 6-(3-methoxypropylamino)-5-nitropicolinate (I-11) as a yellow solid. m/z (ESI) found 284, [M+H]⁺.

### Intermediate 13 (I-13) Procedure J

### Ethyl 2-(3-cyanophenyl)-3-(3-methoxypropyl)-3H-imidazo[4,5-b]pyridine-5-carboxylate

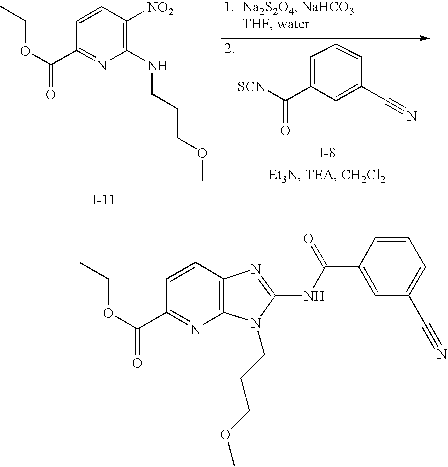

To a mixture of 0.55 g (1.9 mmol, 1.0 eq.) of ethyl 6-(3-methoxypropyl amino)-5-nitropicolinate (I-11) in 100 mL of 1:1 v/v THF:water was added 3.3 g (tech. grade ˜85%, ˜19 mmol, ˜10 eq.) of sodium hydrosulfite and 1.6 g (19 mmol, 10 eq.) of NaHCO₃. The resulting mixture was stirred at room temperature for 1 h. The layers were separated and the organic layer was washed with sat. brine and then dried (Na₂SO₄). The volatiles were removed in vacuo. To a solution of the resulting residue in 100 mL of CH₂Cl₂ was added 0.46 g (2.4 mmol, 1.3 eq.) of 3-cyanobenzoyl isothiocyanate (I-8). The mixture was stirred for 10 min and 0.73 g (3.8 mmol, 2.0 eq.) of EDC and 0.79 mL (5.1 mmol, 3.0 eq.) of triethylamine was added. The reaction mixture was stirred at room temperature for 16 h, and then diluted with sat. NaHCO₃ solution. The organic layer was separated and dried over Na₂SO₄ and the volatiles were removed in vacuo. The resulting residue was purified by flash chromatography (CH₂Cl₂ to 5% MeOH/CH₂Cl₂) to afford ethyl 2-(3-cyanophenyl)-3-(3-methoxypropyl)-3H-imidazo[4,5-b]pyridine-5-carboxylate (I-13). (δ_{H}, 300 MHz, CDCl₃) 1.43 (t, 3H), 2.23 (m, 2H), 3.28 (s, 3H), 3.50 (t, 2H), 4.46 (q, 2H), 4.55 (t, 2H), 7.55 (t, 1H), 7.62 (d, 1H), 7.88 (d, 1H), 8.08 (d, 1H), 8.54 (d, 1H), 8.66 (s, 1H). m/z (ESI) found 408, [M+H]⁺

### Analysis: Analytical HPLC Analysis:

Method A: Waters Millenium 2690/996PDA separations system employing a Phenomonex Luna 3u C8 50×4.6 mm analytical column. The aqueous acetonitrile based solvent gradient involves;
0-1 min—Isocratic 10% of (0.1% TFA/acetonitrile); 1 min-7 min—Linear gradient of 10-90% of (0.1% TFA/acetonitrile): 7 min-9 min—Isocratic 90% of (0.1% TFA/acetonitrile); 9 min-10 min—Linear gradient of 90-10% of (0.1% TFA/acetonitrile); 10 min-12 min—Isocratic 10% of (0.1% TFA/acetonitrile). Flow rate=1 mL/min

Method B: Waters Millenium 2690/996PDA separations system employing a Phenomenex Columbus 5u C18 column 50×4.60 mm analytical column. The aqueous acetonitrile based solvent gradient involves;
0-0.5 min—Isocratic 10% of (0.05% TFA/acetonitrile); 0.5 min-5.5 min—Linear gradient of 10-90% of (0.05% TFA/acetonitrile): 5.5 min-7.5 min—Isocratic 90% of (0.05% TFA/acetonitrile); 7.5 min-8 min—Linear gradient of 90—10% of (0.05% TFA/acetonitrile); 8 min-10 min—Isocratic 10% of (0.05% TFA/acetonitrile). Flow rate=0.4 mL/min

Method C: Waters Millenium 600/996PDA separations system employing a Waters Sunfire 5u C18 column 100×4.60 mm analytical column. The aqueous acetonitrile based solvent gradient involves;
0 min-5.5 min—Linear gradient of 20-90% of (0.05% TFA/acetonitrile): 5.5 min-7.0 min—Isocratic 90% of (0.05% TFA/acetonitrile); 7.0 min-8 min—Linear gradient of 90—20% of (0.05% TFA/acetonitrile); 8 min-10 min—Isocratic 20% of (0.05% TFA/acetonitrile). Flow rate=1.0 ml/min

### Analysis: Mass Spectroscopy

Mass Spectroscopy was conducted using a Thermo-electron LCQ classic or an Applied Biosciences PE Sciex API150ex. Liquid Chromatography Mass Spectroscopy was conducted using a Waters Millenium 2690/996PDA linked Thermo-electron LCQ classic.

### Analysis: NMR Spectroscopy

¹H NMR spectroscopy was conducted using a Varian 300 MHz Gemini 2000, a Bruker 300 MHz AVANCE 300 or Bruker 400 MHz AVANCE^{II} 400 FTNMR.

### A2a Binding Assay:

Membranes prepared from HEK-293 cells that express human A₂ₐ (0.04 mg/mL final, PerkinElmer Life and Analytical Sciences, Boston, Mass.) were mixed with yttrium oxide wheatgerm-agglutinin (WGA)-coated SPA beads (4 mg/mL final, Amersham Biosciences, Piscataway, N.J.) and adenosine deaminase (0.01 mg/ml final) in assay buffer (Dulbecco's phosphate-buffered saline containing 10 mM MgCl₂) for 15 minutes at 4° C. This mixture (10 μL) was added with continuous agitation to the test compounds (10 μL) prepared in 2.5% DMSO or to 2.5% DMSO (1% final) in 384-well assay plates (Corning #3710).

Binding was initiated with the addition of 5 μL [³H]-SCH 58261 (2 nM final, Amersham Biosciences) immediately followed by centrifugation at 1000 rpm for 2 min. The assay plates were incubated in the dark, overnight at room temperature and the signal was detected using a ViewLux CCD Imager (PerkinElmer). Compounds were tested at 11 different concentrations ranging from 0.1 nM to 10 μM. Nonspecific binding was determined in the presence of 10 μM CGS 15943. Assays were performed in duplicate and compounds were tested at least twice. The data were fit to a one-site competition binding model for IC₅₀ determination using the program GraphPad Prism (GraphPad Software, Inc., San Diego, Calif.) and Kᵢ values were calculated using the Cheng-Prusoff equation (Cheng, Y, Prusoff, W. H. Biochem. Pharmacol. 1973, 22, 3099).

### A₁ Binding Assay:

As described in Matasi et al. (Bioorg. Med. Chem. Lett. 2005, 15, 1333), membranes (10 μg) prepared from CHO (Chinese Hamster Ovary) cells that express human A₁ were mixed with 1 nM (final) [³H]-DPCPX in 200 μL assay buffer (2.7 mM KCl, 1.1 mM KH₂PO₄, 137 mM NaCl, 7.6 mM Na₂HPO₄, 10 mM MgCl₂, 0.04% methyl cellulose, 20 ug/mL adenosine deaminase) containing 4% DMSO with or without test compounds. Reactions were carried out for 60 min at room temperature and were terminated by rapid filtration over GF/B filters. Filters were washed seven times with 1 mL cold distilled H₂O, air dried, and radioactivity retained on filters were counted in a Packard TopCount® NXT microplate scintillation counter (Global Medical Instrumentation, Inc., Ramsey, Minn.). Compounds were tested at 10 different concentrations ranging from 0.1 nM to 10 μM. Nonspecific binding was determined in the presence of 10 μM NECA (5′-(N-Ethylcarboxamido)adenosine). Assays were preformed in duplicate and compounds were tested two times. Data were fit to a one-site competition binding model for IC₅₀ determination using the program GraphPad Prism (GraphPad Software, Inc., San Diego, Calif.) and Kᵢ values were calculated using the Cheng-Prusoff equation (Cheng, Y, Prusoff, W. H. Biochem. Pharmacol. 1973, 22, 3099).

Compounds are considered active if they exhibit K for the A₂ₐ receptor less than 10 μM. All the compounds of the examples below exhibited Kᵢ for the A₂ₐ receptor less than 10 μM.

| Compound | hplc (min)/Method | m/z [M + H] |
|---|---|---|
| 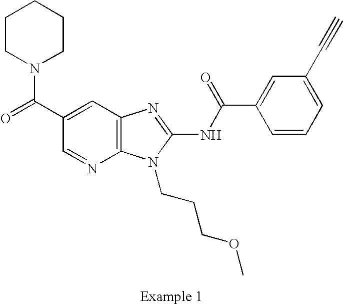 | 6.99 min/Method B | 446.2 |
| 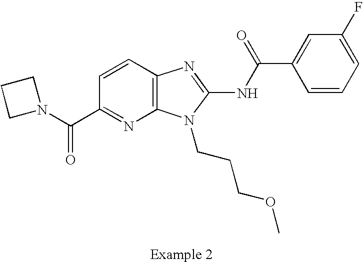 | 6.7 min/Method B | 412.2 |
| 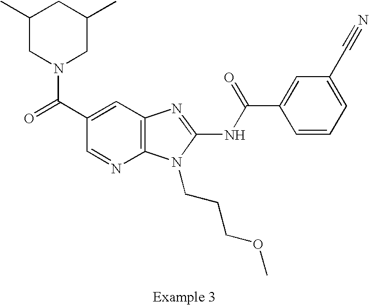 | 7.40 min/Method B | 475.2 |
| 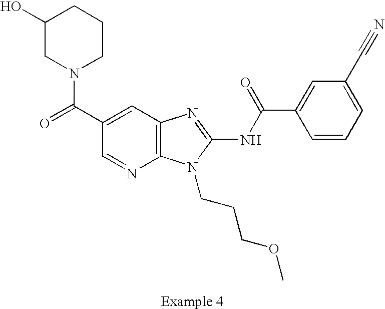 | 5.84 min/Method B | 463.2 |
| 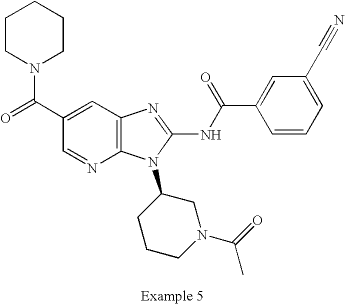 | 6.21 min/Method B | 500.1 |
| 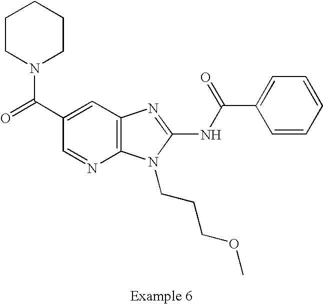 | 6.51 min/Method B | 422.2 |
| 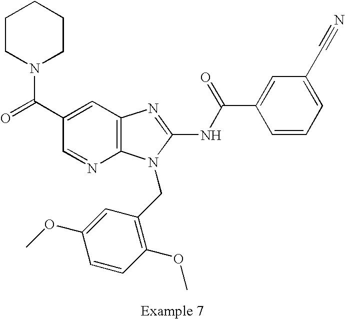 | 7.26 min/Method B | 525.1 |
| 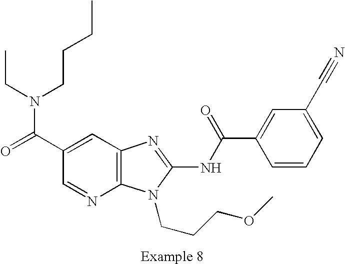 | 7.03 min/Method B | 463.2 |
| 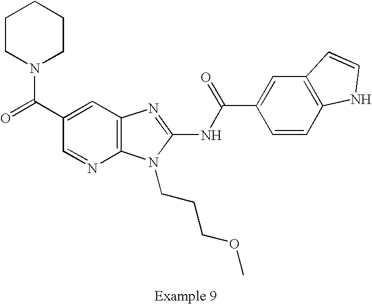 | 5.40 min/Method C | 461.4 |
| 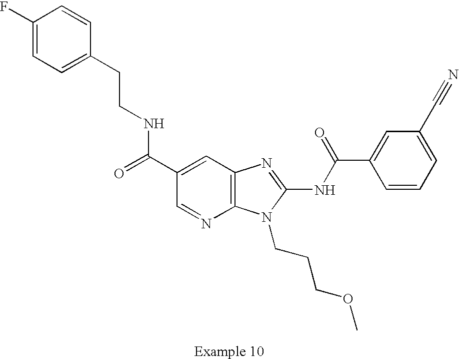 | 6.89 min/Method B | 501.2 |
| 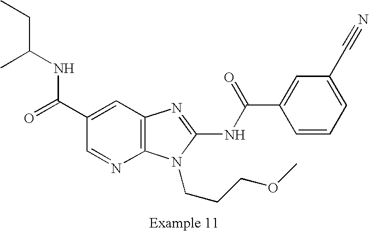 | 6.63 min/Method B | 435.2 |
| 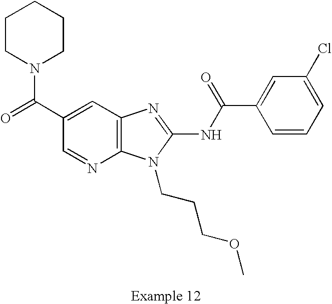 | 7.19 min/Method B | 456.2 |
| 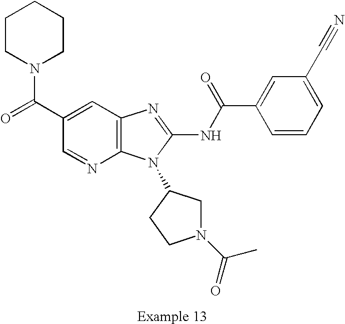 | 5.83 min/Method B | 486.1 |
| 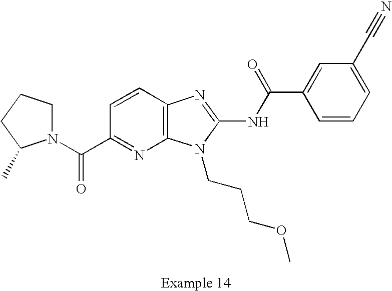 | 6.72 min/Method A | 447.1 |
| 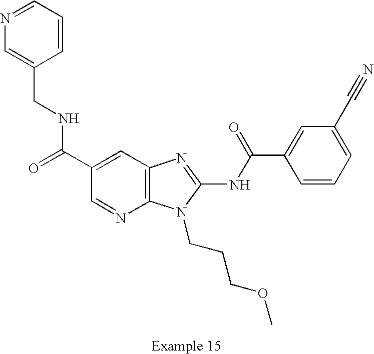 | 4.84 min/Method A | 470.3 |
| 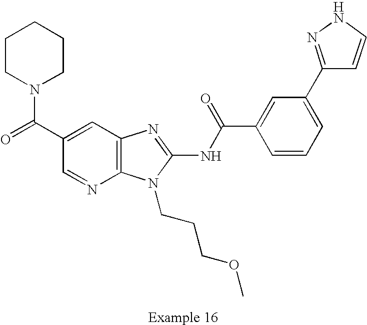 | 5.89 min/Method B | 488.2 |
| 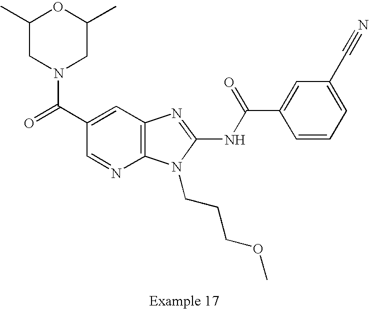 | 6.42 min/Method B | 477.1 |
| 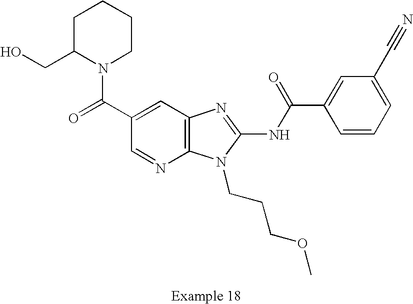 | 5.51 min/Method B | 477.1 |
| 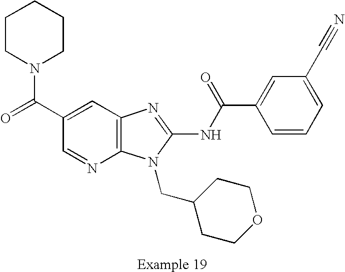 | 6.60 min/Method B | 473.2 |
| 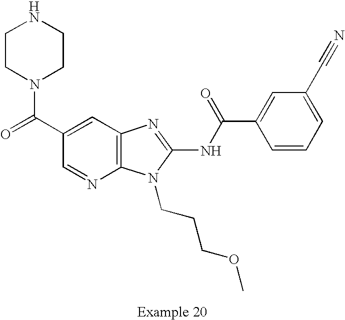 | 4.55 min/Method A | 448.3 |
| 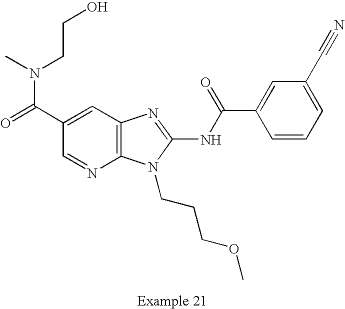 | 5.19 min/Method B | 437.1 |
| 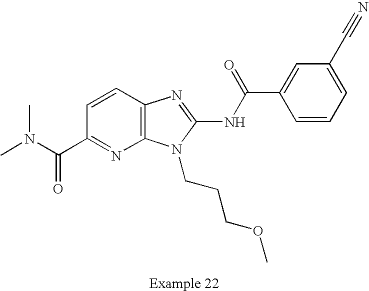 | 5.81 min/Method A | 407.1 |
| 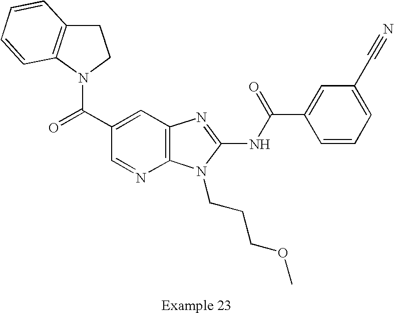 | 7.16 min/Method B | 481.1 |
| 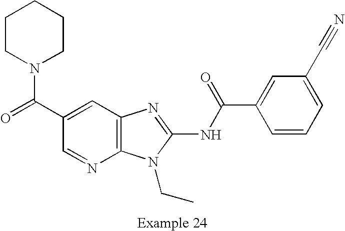 | 6.76 min/Method B | 403.2 |
| 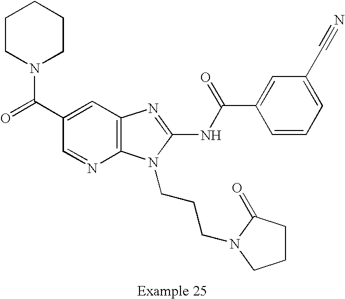 | 5.97 min/Method B | 500.2 |
| 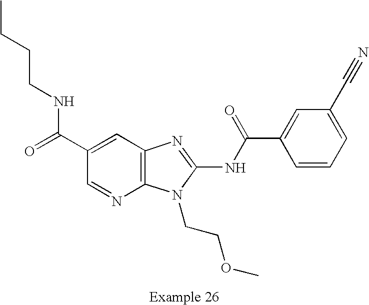 | 6.45 min/Method A | 421.2 |
| 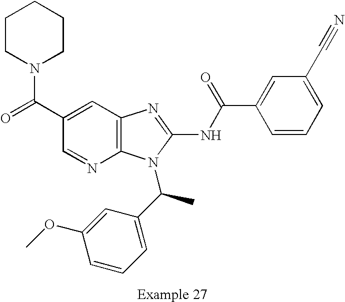 | 7.62 min/Method B | 509.1 |
| 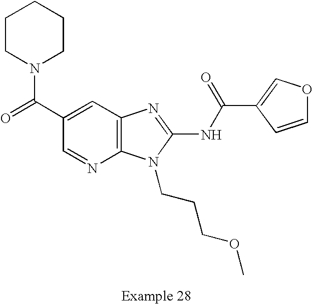 | 5.66 min/Method C | 412.4 |
| 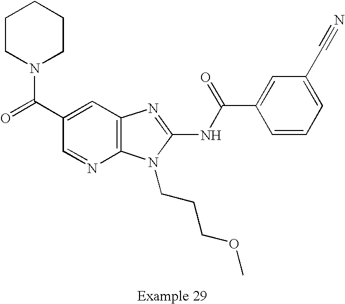 | 5.93 min/Method A | 447.2 |
| 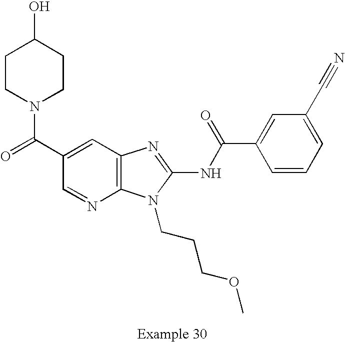 | 5.53 min/Method B | 463.2 |
| 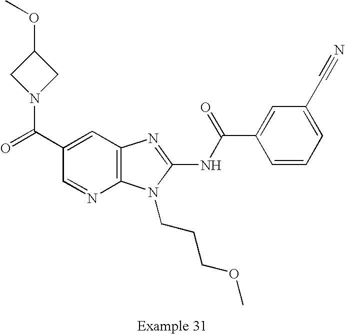 | 6.07 min/Method B | 449.1 |
| 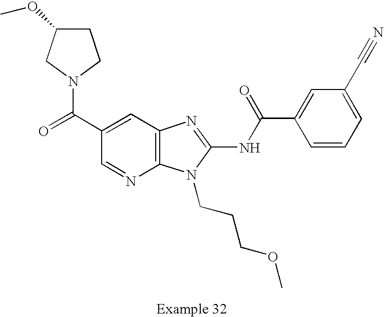 | 6.04 min/Method B | 463.2 |
| 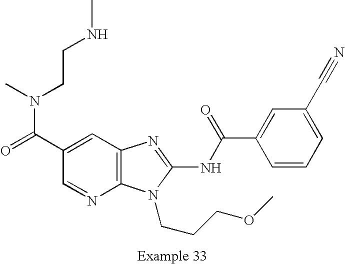 | 4.81 min/Method B | 450.1 |
| 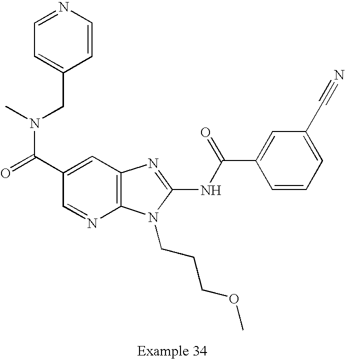 | 4.89 min/Method B | 484.2 |
| 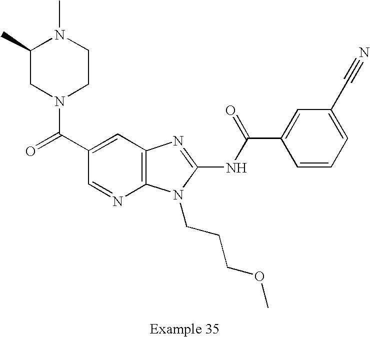 | 5.17 min/Method B | 476.2 |
| 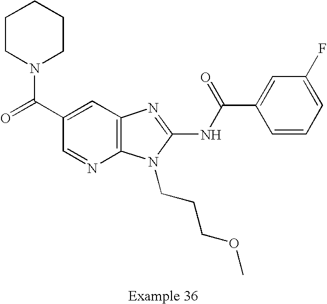 | 6.80 min/Method B | 440.2 |
| 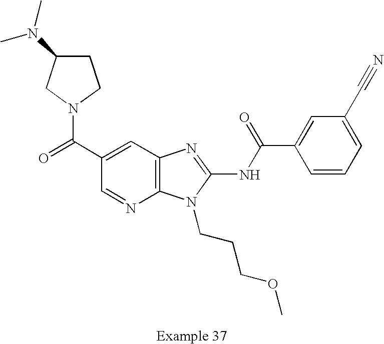 | 4.92 min/Method B | 476.1 |
| 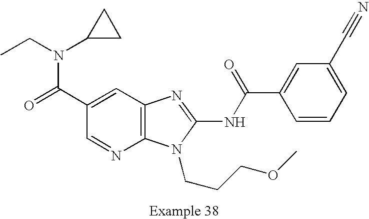 | 6.71 min/Method B | 447.2 |
| 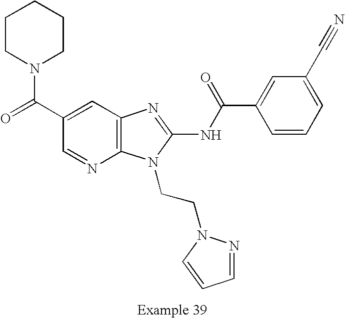 | 6.15 min/Method B | 469.2 |
| 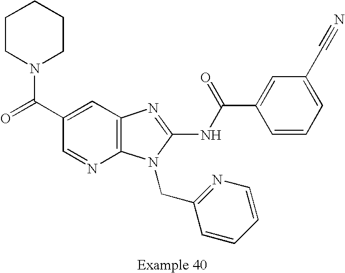 | 6.06 min/Method B | 466.2 |
| 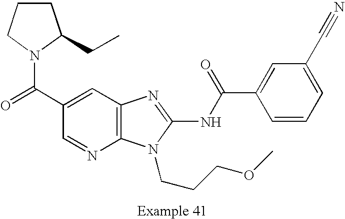 | 6.92 min/Method B | 461.2 |
| 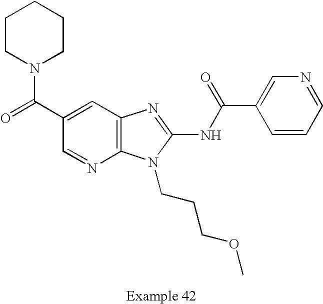 | 4.75 min/Method B | 423.2 |
| 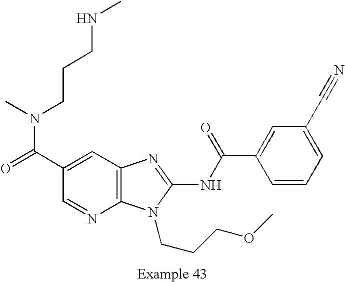 | 4.86 min/Method B | 464.2 |
| 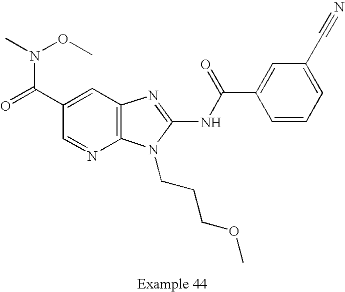 | 6.31 min/Method B | 423.1 |
| 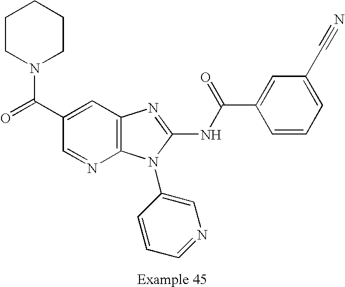 | 5.78 min/Method B | 452.2 |
| 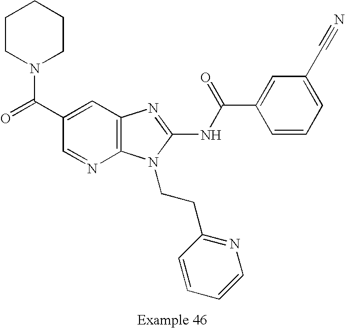 | 5.15 min/Method B | 480.0 |
| 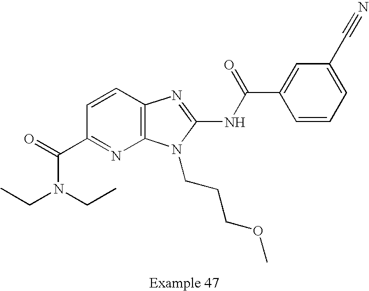 | 6.63 min/Method A | 435.1 |
| 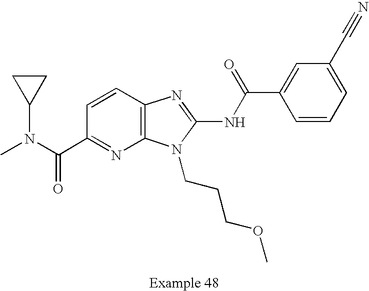 | 6.32 min/Method A | 433.1 |
| 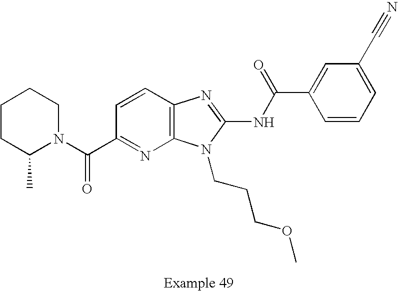 | 6.96 min/Method A | 461.2 |
| 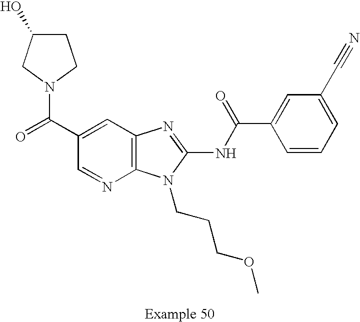 | 5.49 min/Method B | 449.1 |
| 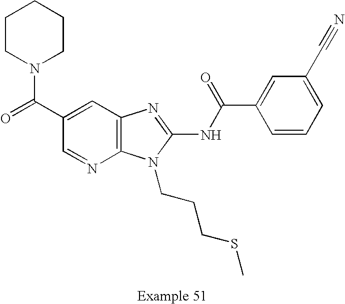 | 7.13 min/Method B | 463.1 |
| 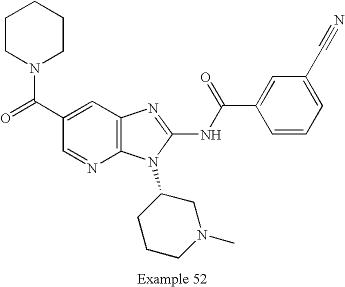 | 5.19 min/Method B | 472.2 |
| 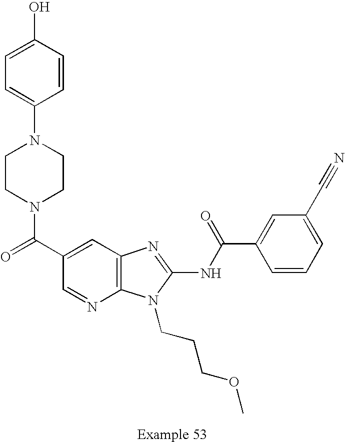 | 5.42 min/Method B | 540.1 |
| 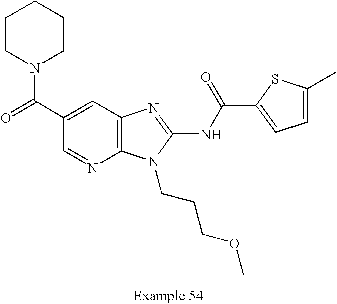 | 6.74 min/Method C | 442.5 |
| 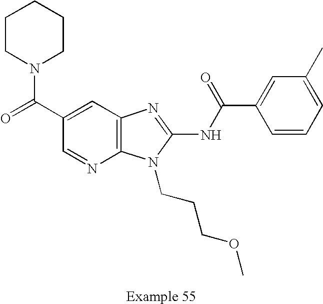 | 6.86 min/Method B | 436.2 |
| 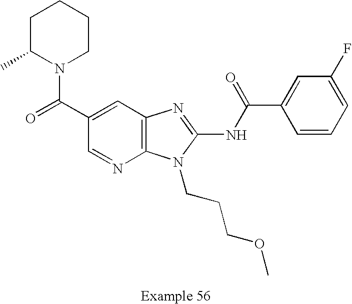 | 7.16 min/Method B | 454.2 |
| 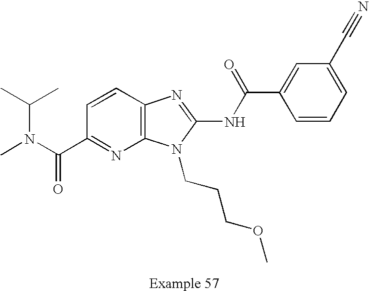 | 6.51 min/Method A | 435.1 |
| 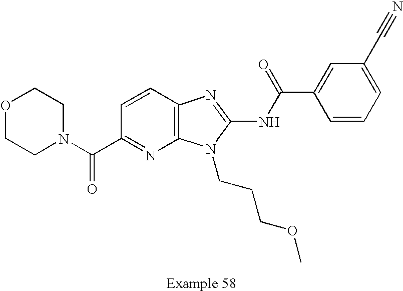 | 6.00 min/Method B | 449.1 |
| 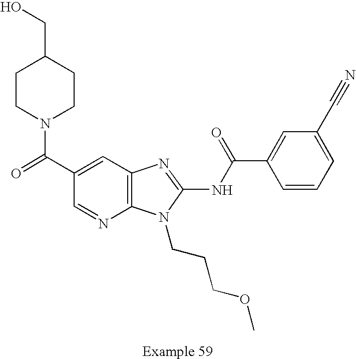 | 5.70 min/Method B | 477.2 |
| 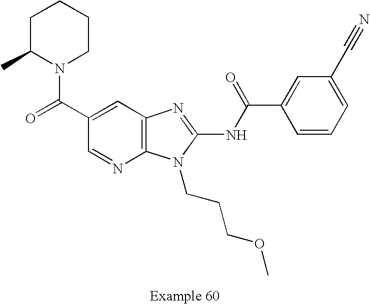 | 6.77 min/Method B | 461.2 |
| 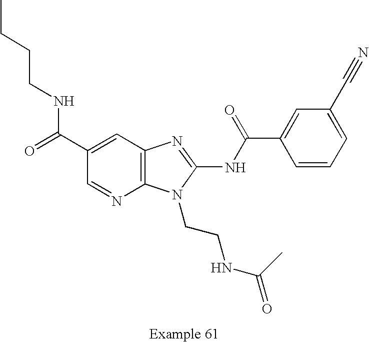 | 5.52 min/Method A | 448.1 |
| 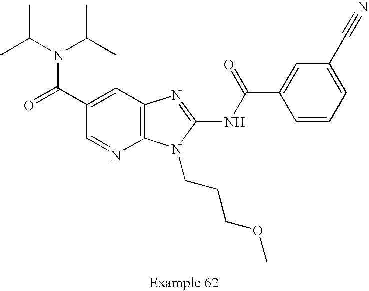 | 7.01 min/Method B | 463.2 |
| 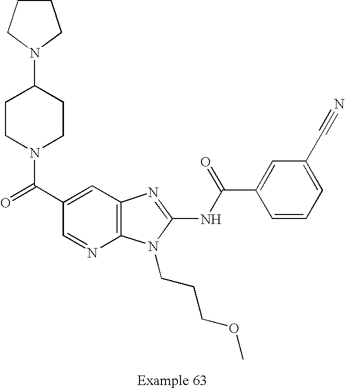 | 5.17 min/Method B | 516.2 |
| 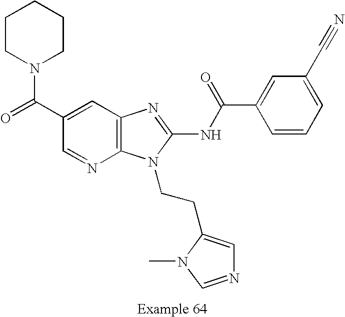 | 5.17 min/Method B | 483.1 |
| 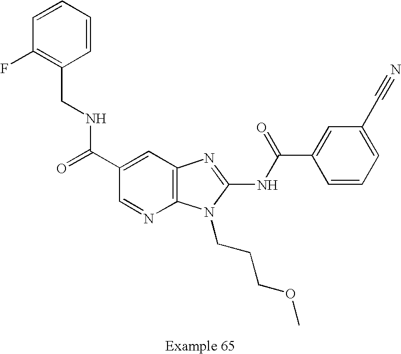 | 6.89 min/Method A | 487.3 |
| 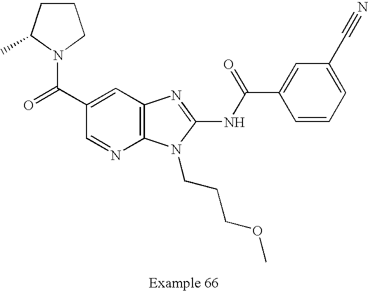 | 6.57 min/Method B | 447.2 |
| 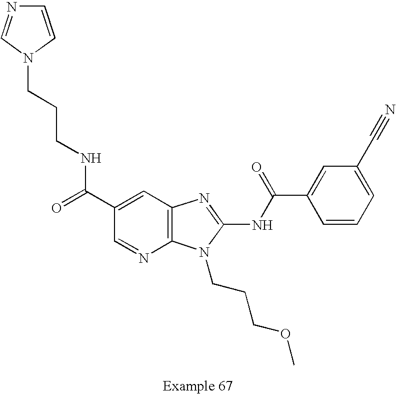 | 4.71 min/Method A | 487.3 |
| 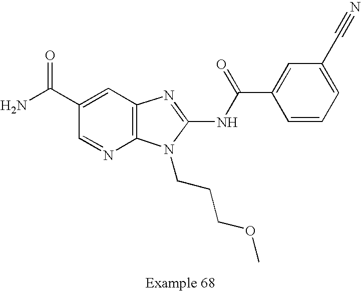 | 5.62 min/Method B | 379.2 |
| 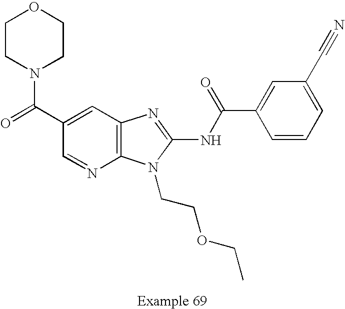 | 5.99 min/Method B | 449.2 |
| 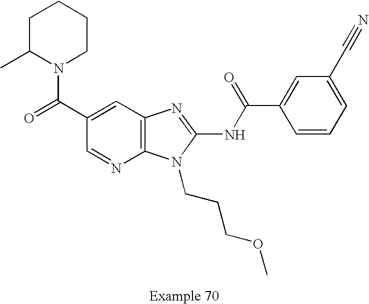 | 6.94 min/Method B | 461.2 |
| 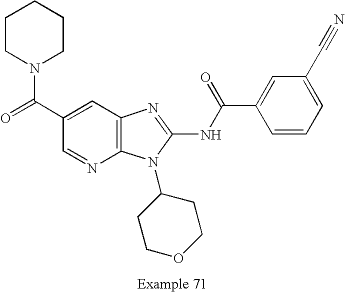 | 6.70 min/Method B | 459.1 |
| 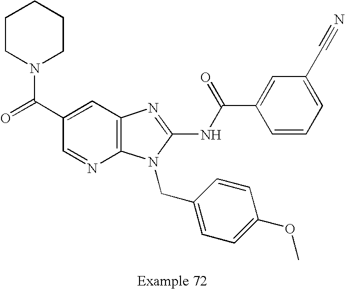 | 7.31 min/Method B | 495.1 |
| 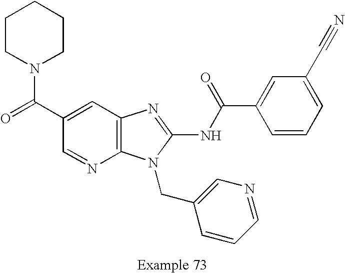 | 5.20 min/Method B | 466.2 |
| 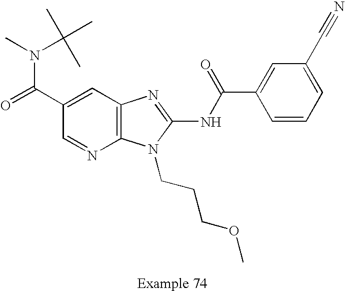 | 7.03 min/Method B | 449.2 |
| 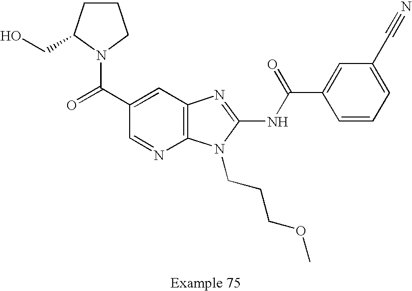 | 5.83 min/Method B | 463.1 |
| 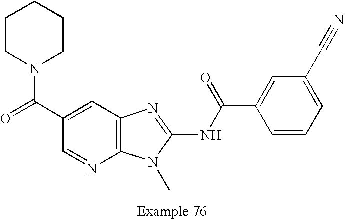 | 6.34 min/Method B | 389.3 |
| 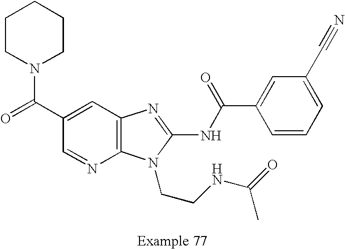 | 5.72 min/Method B | 460.1 |
| 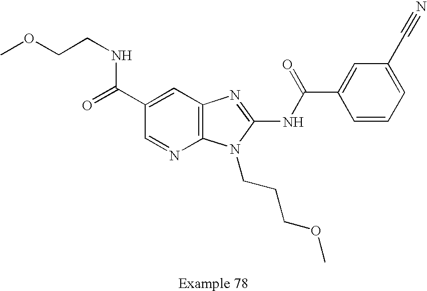 | 5.77 min/Method B | 437.1 |
| 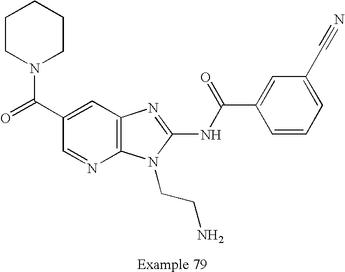 | 4.97 min/Method B | 418.1 |
| 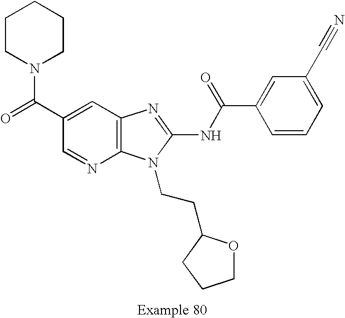 | 6.88 min/Method B | 473.2 |
| 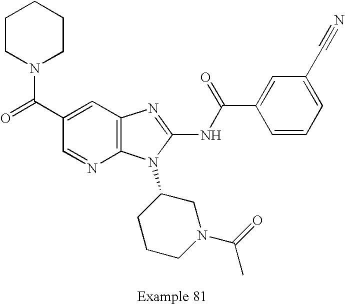 | 6.20 min/Method B | 500.1 |
| 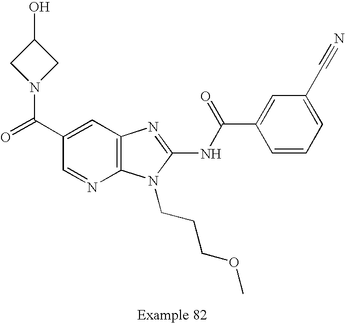 | 5.53 min/Method B | 435.2 |
| 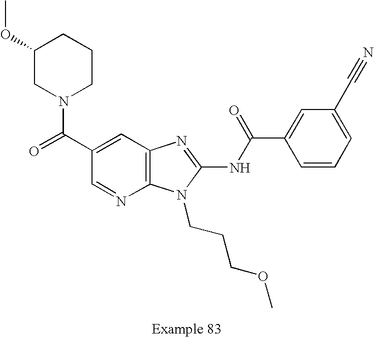 | 6.14 min/Method B | 477.2 |
| 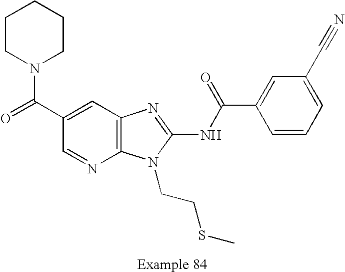 | 6.97 min/Method B | 449.1 |
| 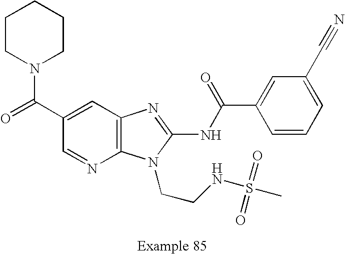 | 5.89 min/Method B | 496.1 |
| 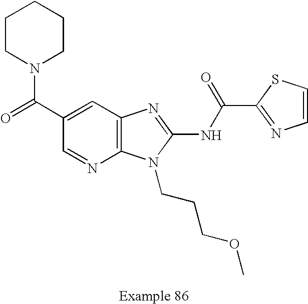 | 5.52 min/Method B | 429.1 |
| 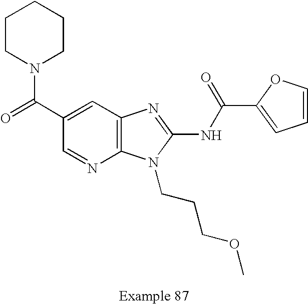 | 5.46 min/Method C | 412.4 |
| 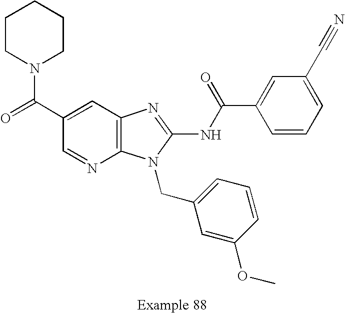 | 7.31 min/Method B | 495.2 |
| 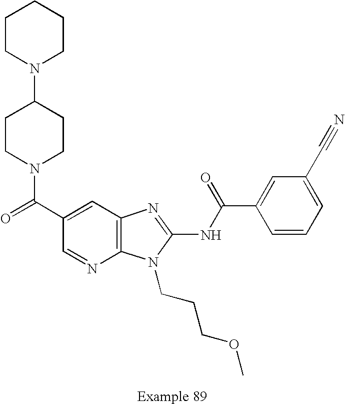 | 5.27 min/Method B | 530 |
| 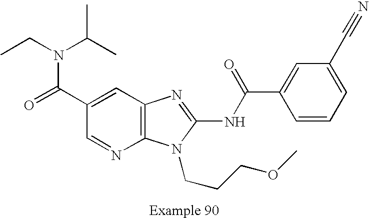 | 6.69 min/Method B | 449.2 |
| 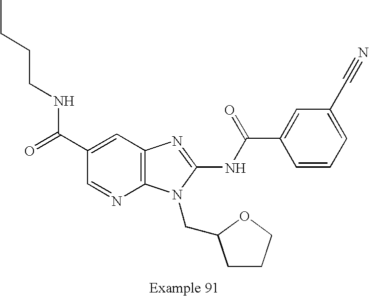 | 6.60 min/Method A | 447.2 |
| 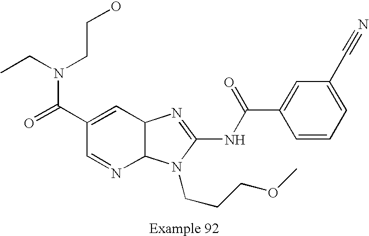 | 6.32 min/Method B | 465.1 |
| 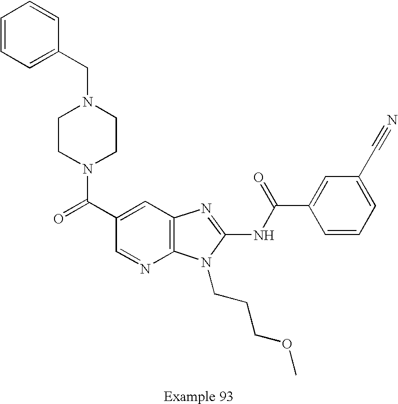 | 5.16 min/Method A | 538.1 |
| 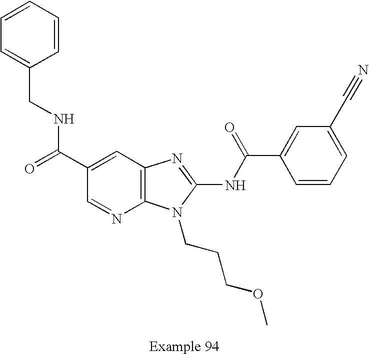 | 6.79 min/Method A | 469.3 |
| 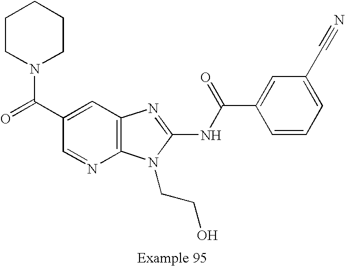 | 5.76 min/Method B | 419.2 |
| 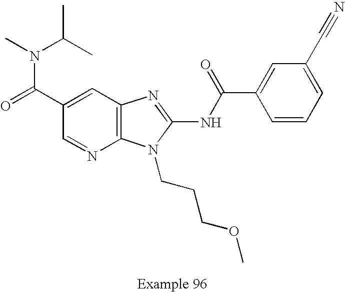 | 6.35 min/Method B | 435.2 |
| 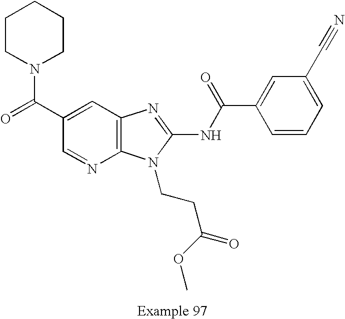 | 6.43 min/Method B | 461.1 |
| 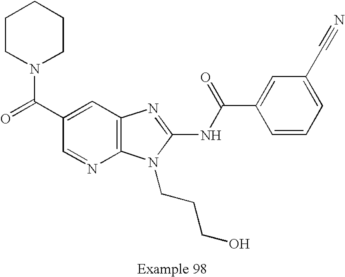 | 5.91 min/Method B | 433.2 |
| 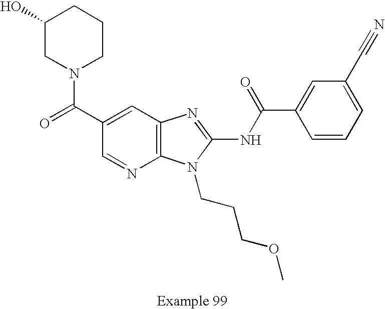 | 5.57 min/Method B | 463.2 |
| 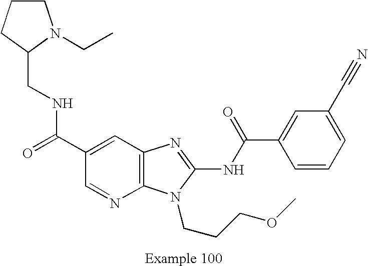 | 4.86 min/Method A | 490.2 |
| 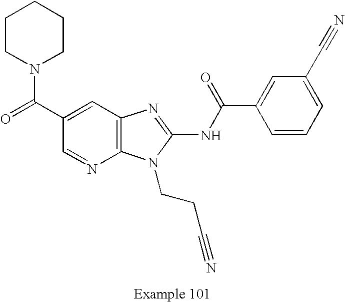 | 6.18 min/Method B | 428.1 |
| 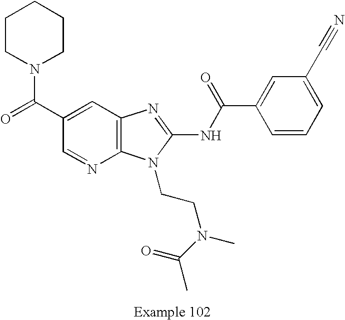 | 5.84 min/Method B | 474.1 |
| 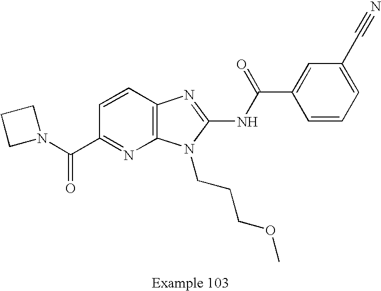 | 6.15 min/Method A | 419.1 |
| 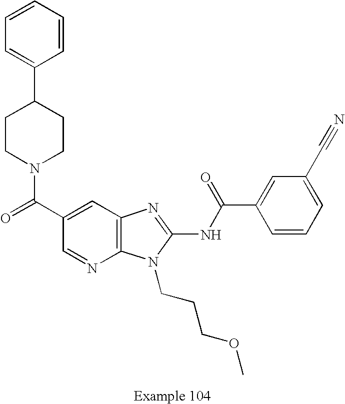 | 7.51 min/Method B | 523.2 |
| 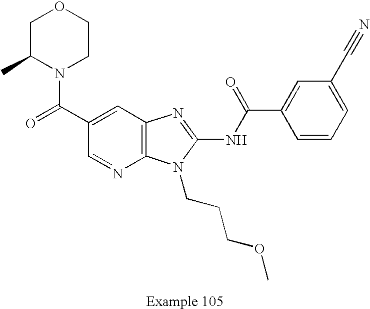 | 6.15 min/Method B | 463.2 |
| 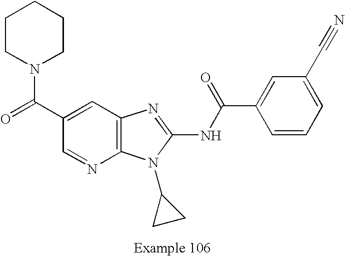 | 6.52 min/Method B | 415.2 |
| 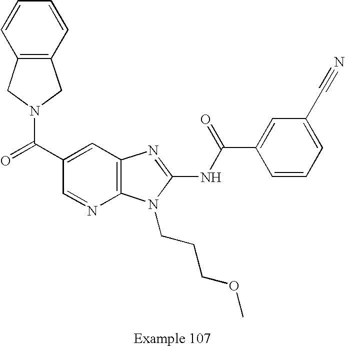 | 6.9 min/Method B | 481.2 |
| 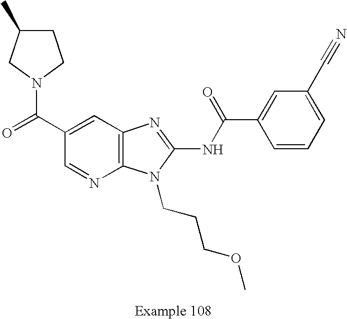 | 6.61 min/Method B | 447.2 |
| 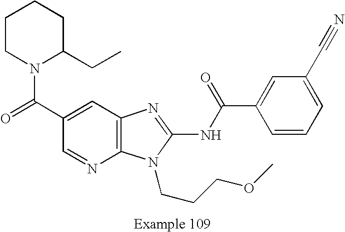 | 7.24 min/Method B | 475.2 |
| 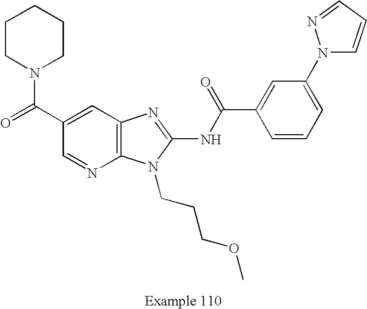 | 6.54 min/Method B | 488.2 |
| 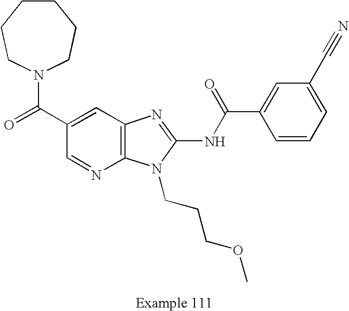 | 6.96 min/Method B | 461.2 |
| 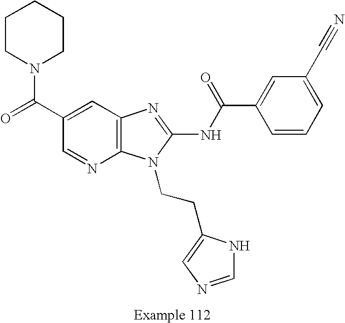 | 5.12 min/Method B | 469.2 |
| 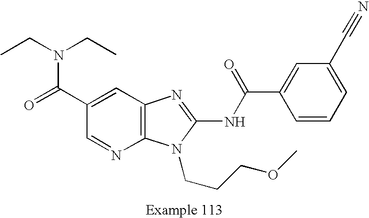 | 6.45 min/Method B | 435.2 |
| 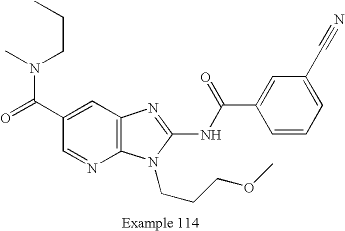 | 6.44 min/Method B | 435.2 |
| 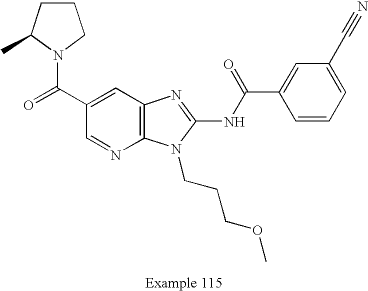 | 6.43 min/Method B | 447.2 |
| 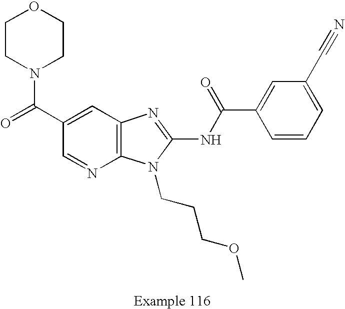 | 5.96 min/Method B | 449.1 |
| 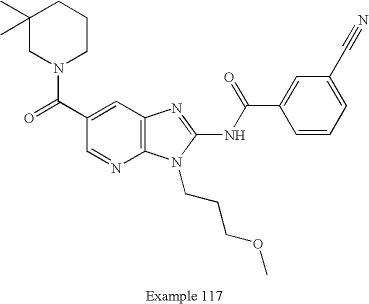 | 7.05 min/Method B | 475.2 |
| 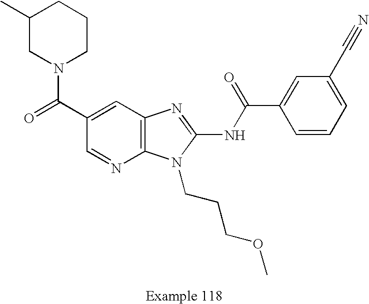 | 7.14 min/Method B | 461.2 |
| 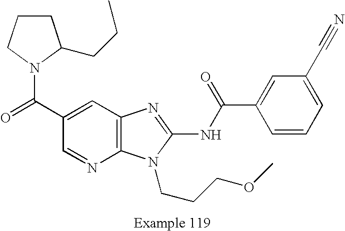 | 7.25 min/Method B | 475.2 |
| 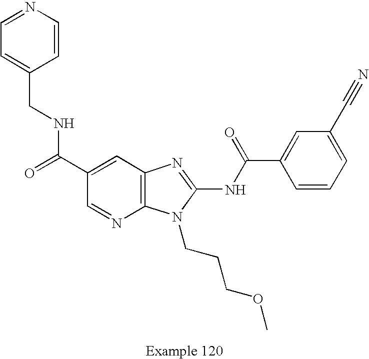 | 4.95 min/Method A | 470.3 |
| 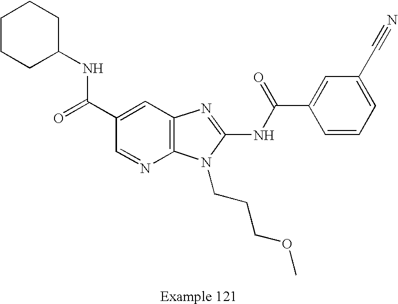 | 7.00 min/Method B | 461.2 |
| 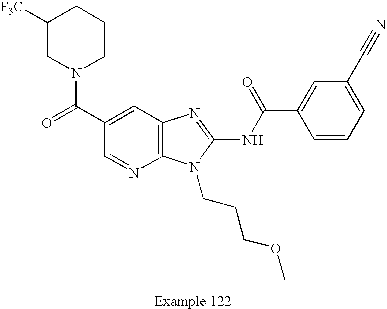 | 6.93 min/Method B | 515.2 |
| 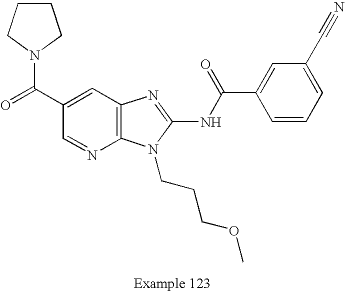 | 5.83 min/Method A | 433.2 |
| 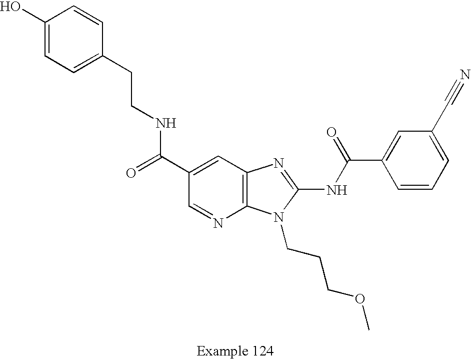 | 6.12 min/Method B | 489.2 |
| 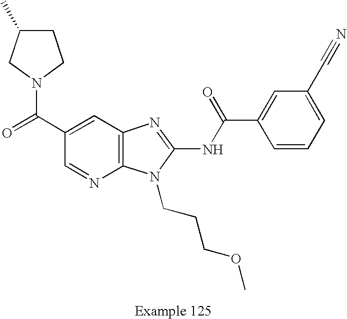 | 6.61 min/Method B | 447.2 |
| 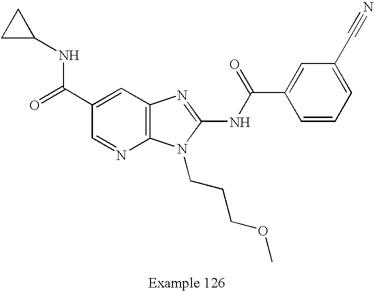 | 5.84 min/Method A | 419.2 |
| 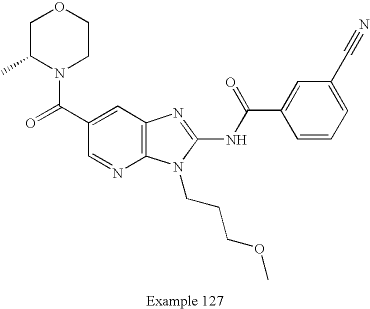 | 6.12 min/Method B | 463.2 |
| 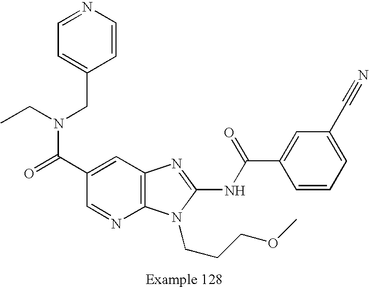 | 5.23 min/Method B | 498.2 |
| 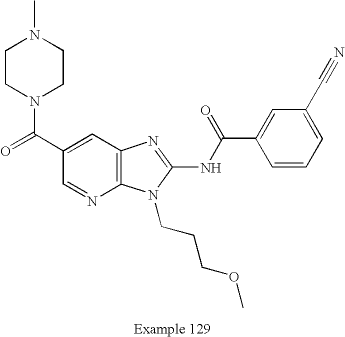 | 4.56 min/Method A | 462.3 |
| 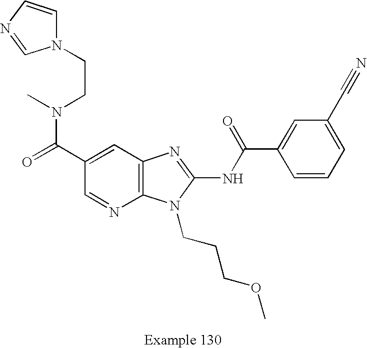 | 5.00 min/Method B | 487 |
| 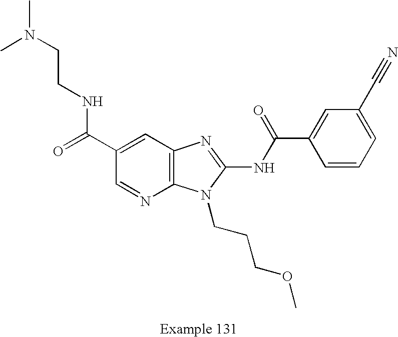 | 4.76 min/Method A | 450.1 |
| 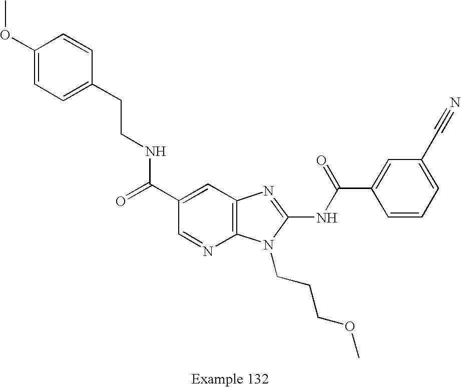 | 6.77 min/Method B | 513.1 |
| 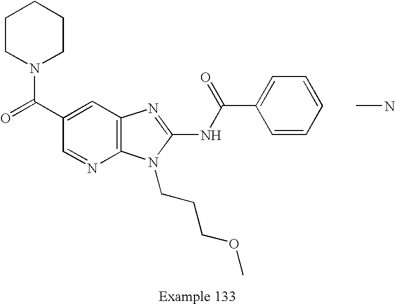 | 6.48 min/Method B | 447.2 |
| 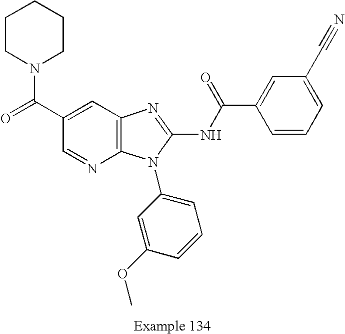 | 7.00 min/Method B | 481.2 |
| 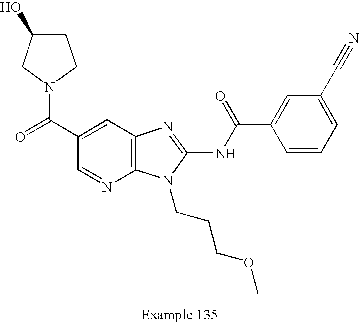 | 5.48 min/Method B | 449.2 |
| 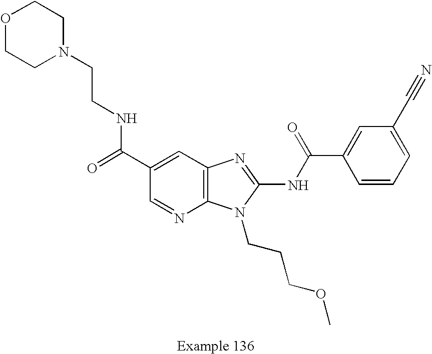 | 4.68 min/Method A | 492.3 |
| 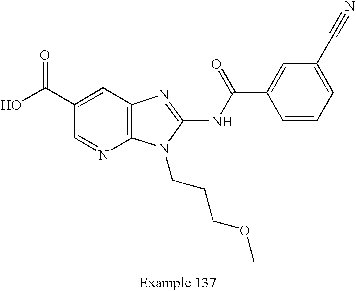 | 5.97 min/Method A | 380.3 |
| 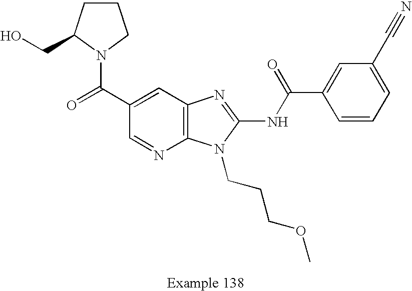 | 5.81 min/Method B | 463.2 |
| 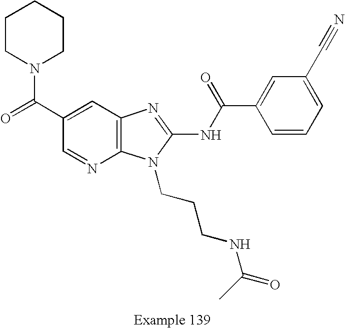 | 5.68 min/Method B | 474.1 |
| 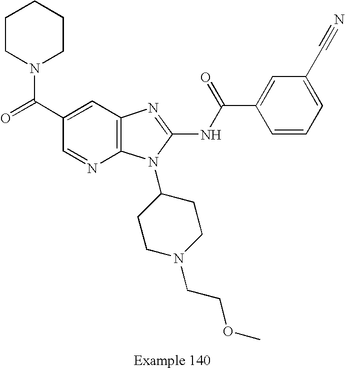 | 5.25 min/Method B | 516.1 |
| 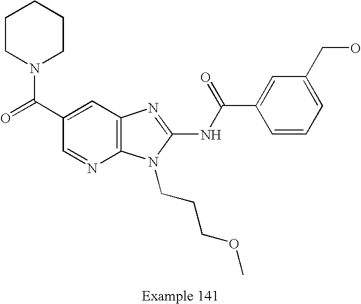 | 6.38 min/Method B | 450.2 |
| 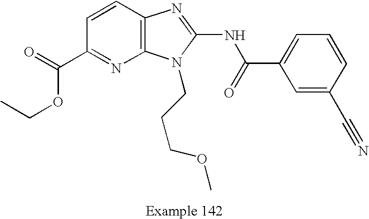 | 6.90 min/Method B | 408.1 |
| 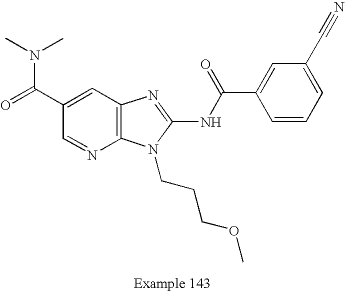 | 5.93 min/Method A | 407.3 |
| 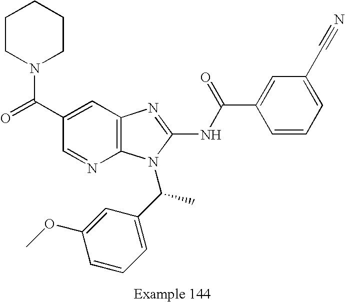 | 7.62 min/Method B | 509.1 |
| 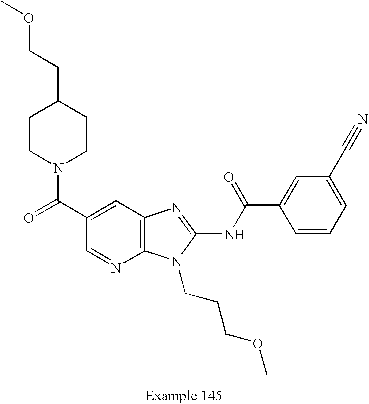 | 6.66 min/Method B | 505.2 |
| 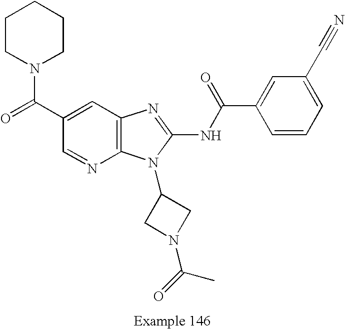 | 5.65 min/Method B | 472.1 |
| 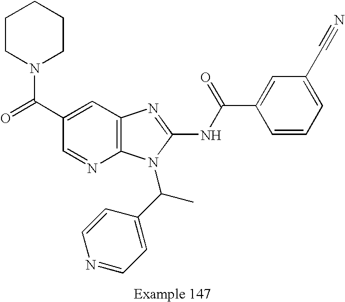 | 5.36 min/Method B | 480.2 |
| 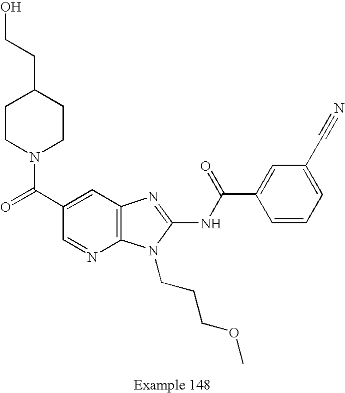 | 5.84 min/Method B | 491.2 |
| 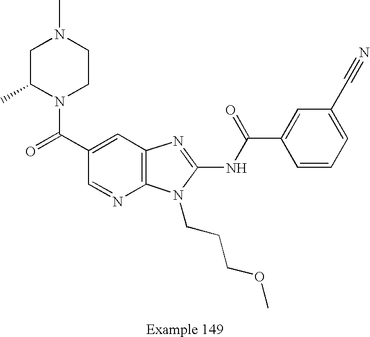 | 5.11 min/Method B | 476.2 |
| 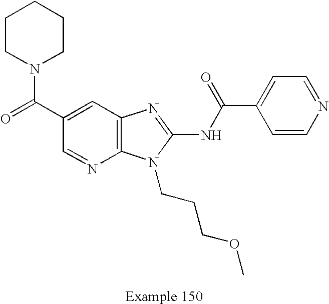 | 4.68 min/Method B | 423.2 |
| 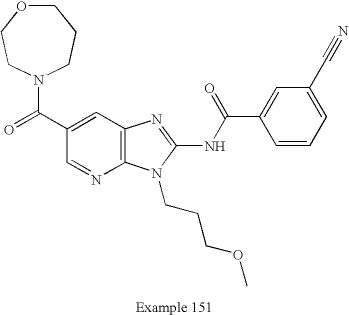 | 5.80 min/Method B | 463.1 |
| 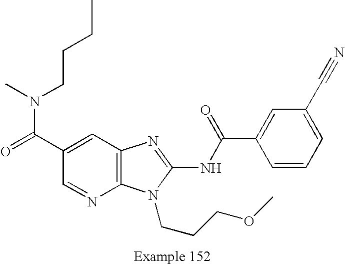 | 6.91 min/Method B | 449.2 |
| 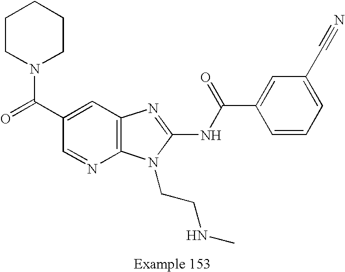 | 5.06 min/Method B | 432.1 |
| 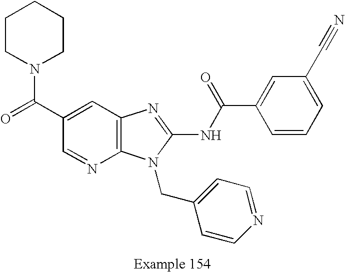 | 5.15 min/Method B | 466.2 |
| 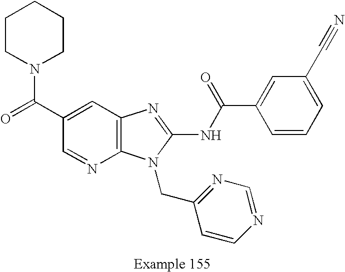 | 5.90 min/Method B | 467.2 |
| 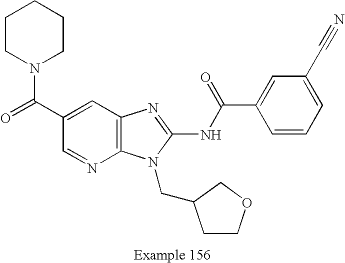 | 6.44 min/Method B | 459.2 |
| 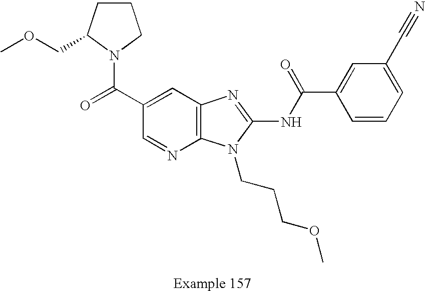 | 6.03 min/Method B | 477.2 |
| 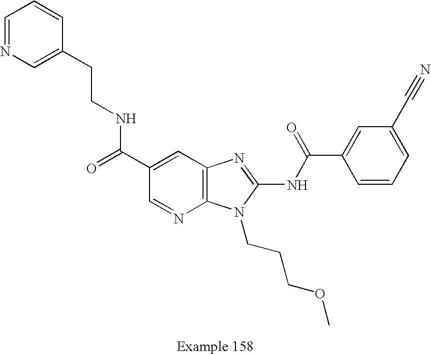 | 4.89 min/Method B | 484.2 |
| 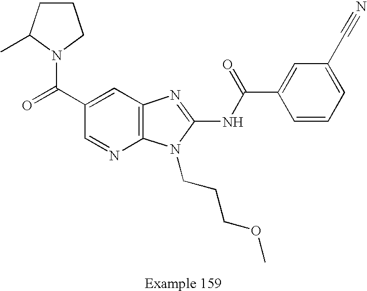 | 6.55 min/Method B | 447.2 |
| 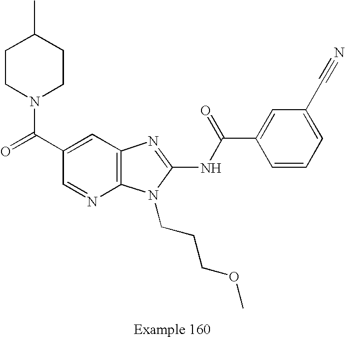 | 6.98 min/Method B | 461.2 |
| 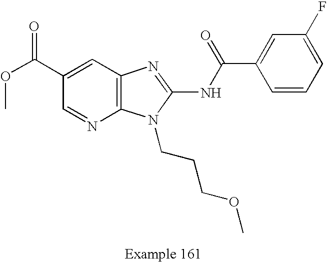 | 7.25 min/Method B | 387.2 |
| 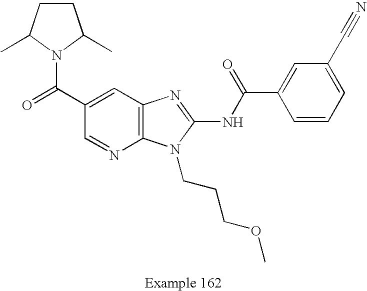 | 6.90 min/Method B | 461.2 |
| 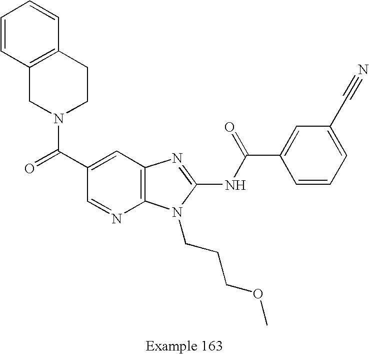 | 7.10 min/Method B | 495.2 |
| 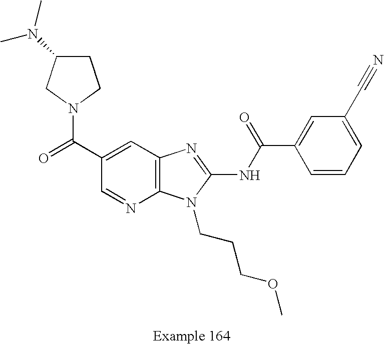 | 4.91 min/Method B | 476.1 |
| 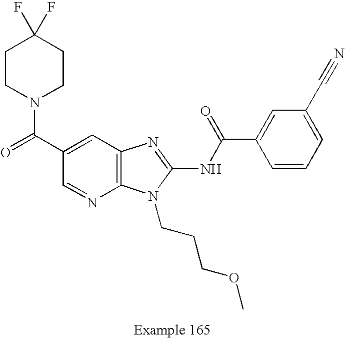 | 6.53 min/Method B | 483.2 |
| 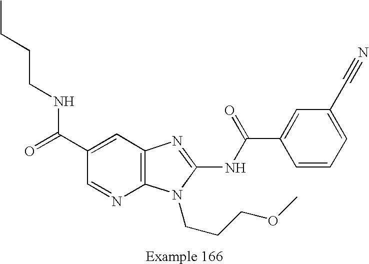 | 6.60 min/Method A | 435.2 |
| 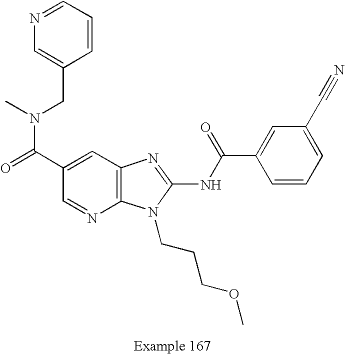 | 4.98 min/Method B | 484.2 |
| 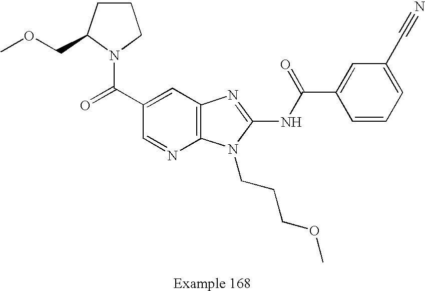 | 6.44 min/Method B | 477.2 |
| 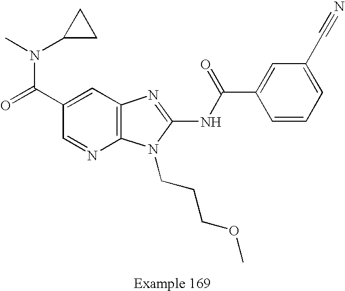 | 6.37 min/Method B | 433.2 |
| 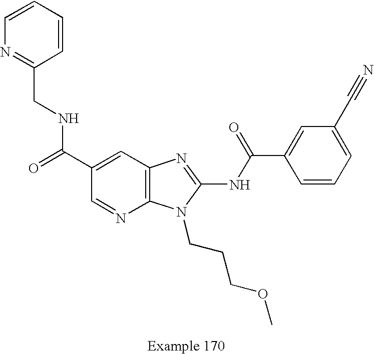 | 4.91 min/Method A | 470.3 |
| 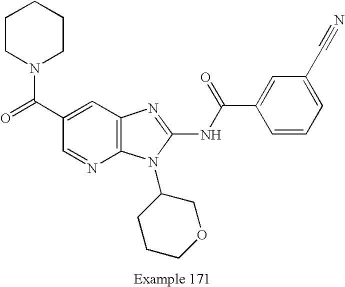 | 6.89 min/Method B | 459.1 |
| 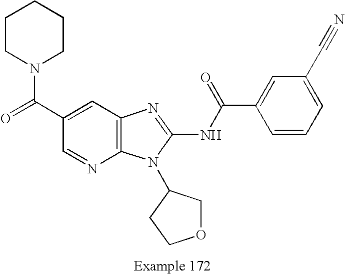 | 6.49 min/Method B | 445.1 |
| 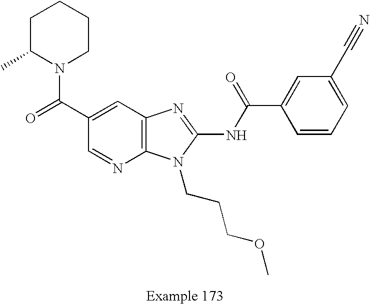 | 6.89 min/Method B | 461.2 |
| 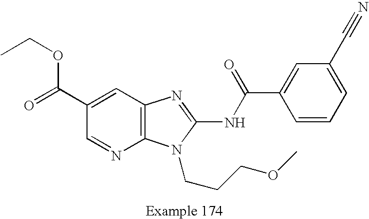 | 7.12 min/Method A | 408.2 |
| 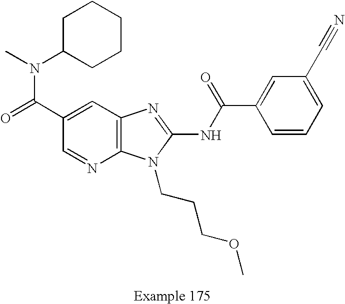 | 7.18 min/Method B | 475.2 |
| 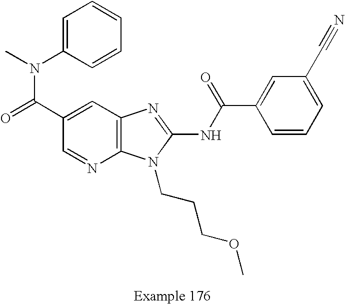 | 6.71 min/Method B | 469.2 |
| 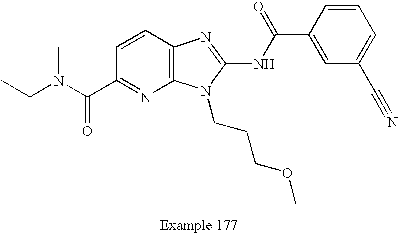 | 6.14 min/Method A | 421.1 |
| 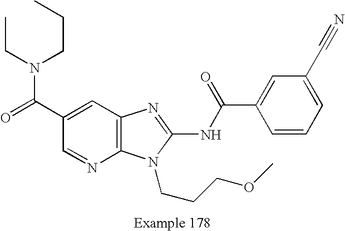 | 6.71 min/Method B | 449.2 |
| 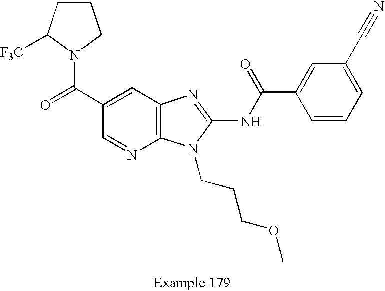 | 6.92 min/Method B | 501.2 |
| 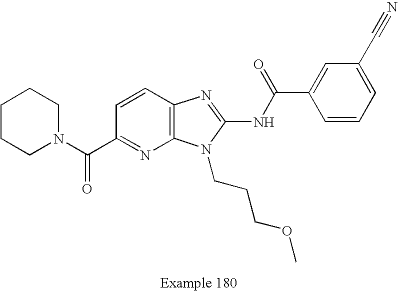 | 6.70 min/Method A | 447.1 |
| 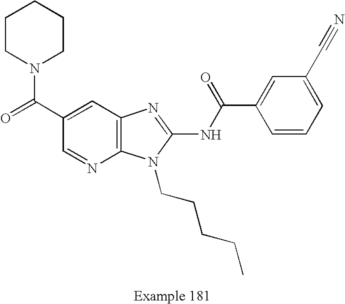 | 7.80 min/Method B | 445.2 |
| 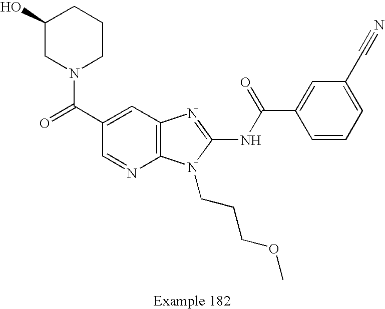 | 5.71 min/Method B | 463.2 |
| 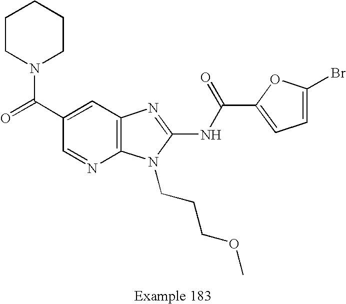 | 6.46 min/Method B | 492.1 |
| 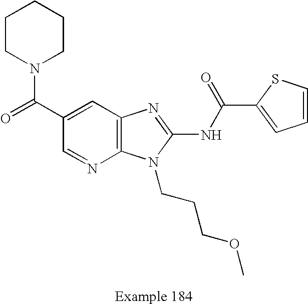 | 6.37 min/Method C | 428.4 |
| 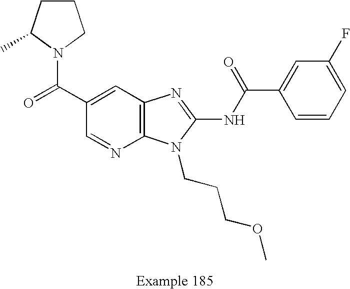 | 6.83 min/Method B | 440.2 |
| 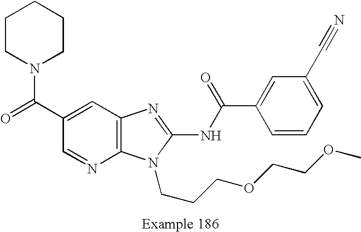 | 6.55 min/Method B | 491.2 |
| 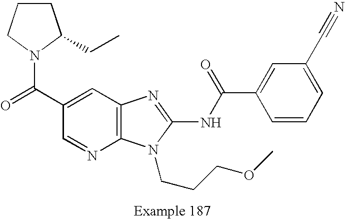 | 6.91 min/Method B | 461.2 |
| 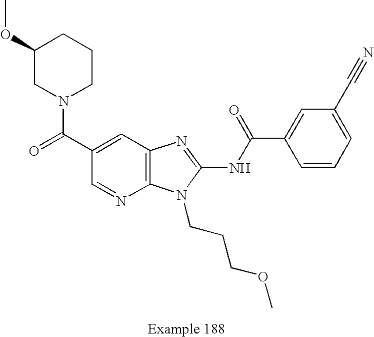 | 6.12 min/Method B | 477.2 |
| 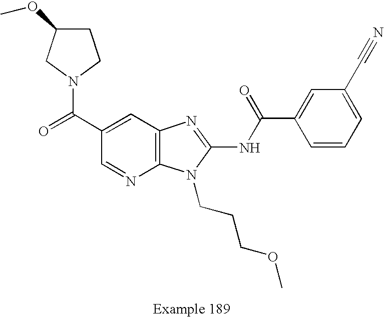 | 6.03 min/Method B | 463.2 |
| 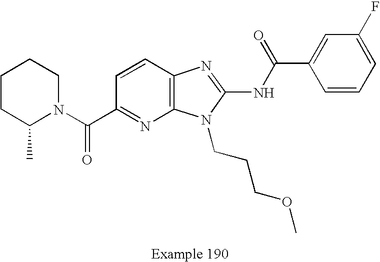 | 7.35 min/Method B | 454.2 |
| 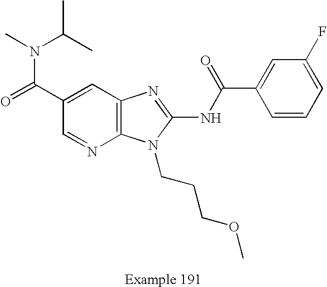 | 6.80 min/Method B | 428.2 |
| 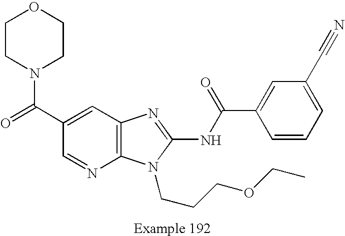 | 6.19 min/Method B | 463.2 |
| 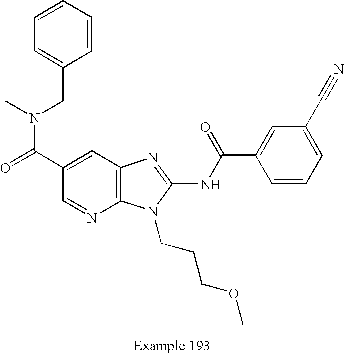 | 6.86 min/Method A | 483.2 |
| 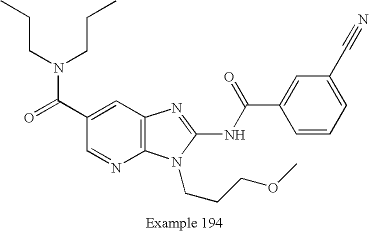 | 7.03 min/Method B | 463.2 |
| 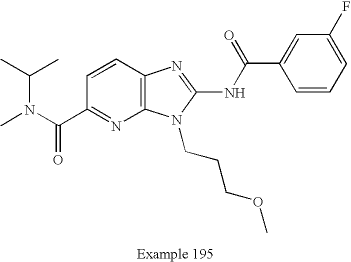 | 6.89 min/Method B | 428.1 |
| 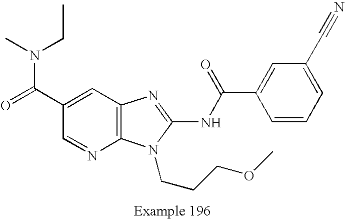 | 6.07 min/Method B | 421.1 |
| 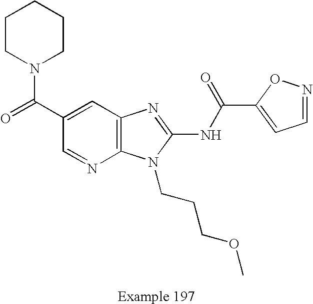 | 5.61 min/Method B | 413.2 |
| 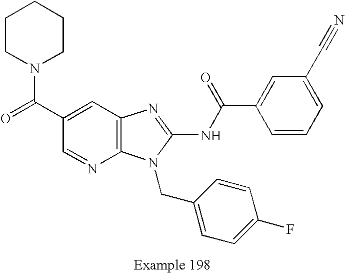 | 7.42 min/Method B | 483.1 |
| 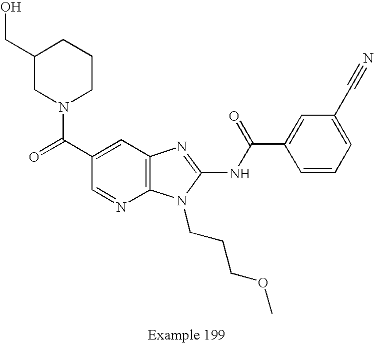 | 5.86 min/Method B | 477.2 |
| 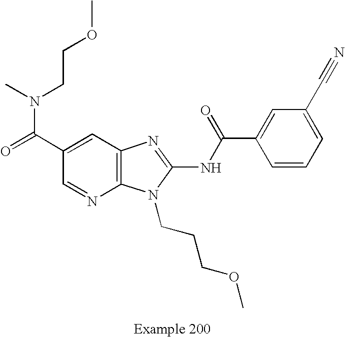 | 5.93 min/Method B | 451.1 |

### 3-Ethynyl-N-(3-(3-methoxypropyl)-6-(piperidine-1-carbonyl)-3H-imidazo[4,5-b]pyridin-2-yl)benzamide

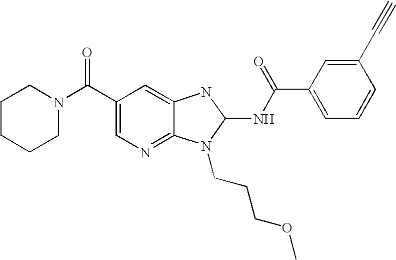

(δ_{H}, 300 MHz, CDCl₃) 1.53 (m, 6H), 2.16 (m, 2H), 3.07 (s, 1H), 3.26 (s, 3H), 3.44 (m, 6H), 4.42 (t, 2H), 7.36 (t, 1H), 7.94 (d, 1H), 7.56 (m, 1H), 8.27 (m, 2H), 8.45 (s, 1H); ESI, 446.2 [M+H].

### N-[6-(Azetidine-1-carbonyl)-3-(3-methoxy-propyl)-3H-imidazo[4,5-b]pyridin-2-yl]-3-fluoro-benzamide

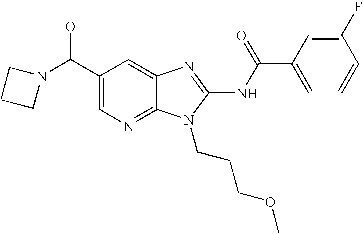

(δ_{H}, 300 MHz, CDCl₃) 2.20 (m, 2H), 2.21 (m, 2H), 3.33 (s, 3H), 3.45 (m, 2H), 4.31 (t, 2H), 4.44 (t, 2H), 4.88 (t, 2H), 7.20 (m, 1H), 7.40 (m, 1H), 7.65 (d, 1H), 7.98 (m, 1H), 8.09 (m, 2H); ESI, 412.2 [M+H].

### (+/−)-3-Cyano-N-[6-(3-hydroxy-piperidine-1-carbonyl)-3-(3-methoxy-propyl)-3H-imidazo[4,5-b]pyridin-2-yl]-benzamide

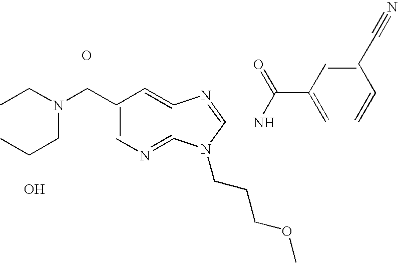

(δ_{H}, CDCl₃) 1.55 (4H), 2.28 (m, 2H), 2.70 (s, 1H), 3.59 (s, 3H), 3.45 (m, 4H), 4.34 (m, 2H), 4.46 (t, 2H), 7.55 (t, 1H), 7.75 (d, 1H), 7.88 (s, 1H), 8.39 (s, 1H), 8.50 (d, 2H), 8.62 (s, 1H); ESI, 463.2 [M+H].

### N-(3-(3-Methoxypropyl)-6-(piperidine 1-carbonyl)-3H-imidazo[4,5-b]pyridin-2-yl)benzamide

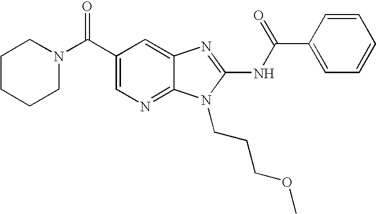

(δ_{H}, 300 MHz, CDCl₃) 150˜1.70 (m, 6H), 2.13 (m, 2H), 3.22 (s, 3H), 3.39 (bt, 2H), 3.43 (t, 2H), 3.68 (bt, 2H), 4.48 (t, 2H), 7.44 (t, 2H), 7.53 (t, 1H), 7.84 (s, 1H), 8.12 (d, 2H), 8.33 (s, 1H); ESI 422.2 [M+H].

### N-Butyl-2-(3-cyano-benzoylamino)-N-ethyl-3-(3-methoxypropyl)-3H-imidazo[4,5-b]pyridine-6-carboxamide

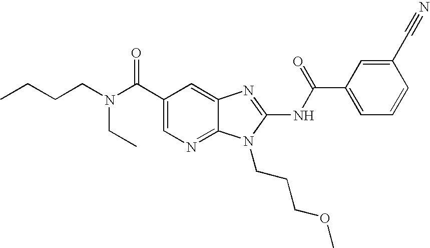

(δ_{H}, 300 MHz, CD₃OD) 0.8˜1.65 (m, 10H), 2.12 (m, 2H), 3.18 (s, 3H), 3.21 (t, 2H), 3.42 (bm, 4H), 4.40 (t, 2H), 7.55 (m, 1H), 7.69 (d, 1H), 7.76 (m, 1H), 8.01 (d, 1H), 8.46 (m, 1H), 8.54 (s, 1H); ESI 463.2 [M+H].

### N-(3-(3-Methoxypropyl)-6-(piperidine-1-carbonyl)-3H-imidazo[4,5-b]pyridin-2-yl)-1H-indole-5-carboxamide

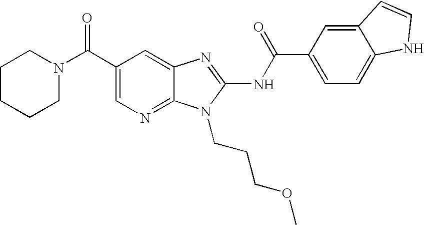

(δ_{H}, 300 MHz, CD₃OD) 1.7˜1.9 (m, 6H), 2.47 (m, 2H), 3.40 (s, 3H), 3.49 (s, 1H), 3.61 (bm, 2H), 3.67 (t, 2H), 3.89 (bm, 2H), 4.74 (t, 2H), 6.78 (d, 1H), 7.52 (d, 1H), 7.64 (d, 1H), 8.09 (d, 1H), 8.13 (d, 1H), 8.58 (d, 1H), 8.66 (s 1H); ESI 461 [M+H].

### N-(4-fluorophenethyl)-2-(3-cyanobenzamido)-3-(3-methoxypropyl)-3H-imidazo[4,5-b]pyridine-6-carboxamide

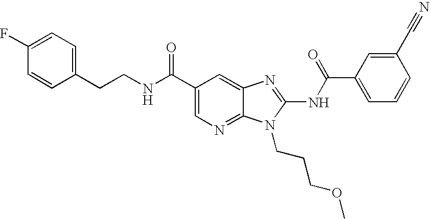

(δ_{H}, 300 MHz, d₆DMSO) 2.00 (m, 2H), 2.77 (t, 2H), 3.10 (s, 3H), 3.34 (t, 2H), 3.39 (t, 2H), 4.28 (t, 2H), 7.02 (t, 2H), 7.19 (m, 2H), 7.64 (t, 1H), 7.92 (d, 1H), 8.12 (s, 1H), 8.42 (d, 1H), 8.50 (s, 1H), 8.59 (d, 1H); ESI 501.2 [M+H].

### 3-Chloro-N-(3-(3-methoxy-propyl)-6-(piperidine-1-carbonyl)-3H-imidazo[4,5-b]pyridine-2-yl)-benzamide

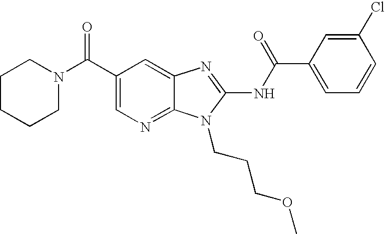

(δ_{H}, 300 MHz, CD₃OD) 1.5˜1.7 (m, 6H), 2.11 (m, 2H), 3.17 (s, 3H), 3.42 (t, 4H), 3.50 (m, 2H), 4.40 (t, 2H), 7.40 (m, 2H), 7.71 (d, 1H), 8.11 (m, 1H), 8.22 (m, 2H); ESI 456.2 [M+H].

### (S)—N43-(˜1-Acetylpyrrolidin-3-yl)-6-(piperidine-1-carbonyl)-3H-imidazo[4,5-b]pyridin-2-yl)-3-cyanobenzamide

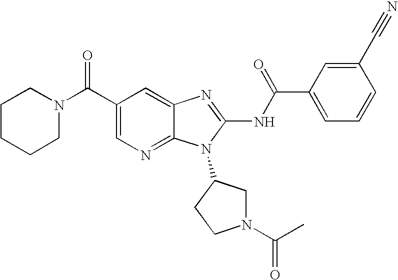

(δ_{H}, 300 MHz, CDCl₃) 1.64 (m, 6H), 2.13 (d, 3H), 2.38 (m, 1H), 3.09 (m, 1H), 3.58 (m, 5H), 3.97 (m, 2H), 4.31 (m, 1H), 5.72 (m, 1H), 7.58 (m, 1H), 7.66 (m, 1H), 7.77 (m, 1H), 8.29 (m, 1H), 8.47 (m, 1H), 8.59 (s, 1H); ESI, 486.1 [M+H].

### N-(3-(3-methoxypropyl)-6-(piperidine-1-carbonyl)-3H-imidazo[4,5-b]pyridin-2-yl)-3-(3H-pyrazol-3-yl)benzamide

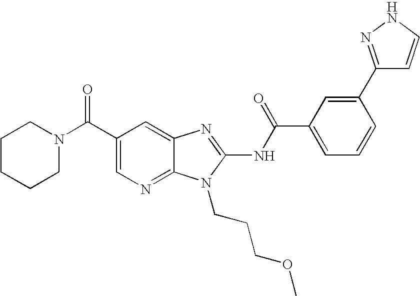

(δ_{H}, 300 MHz, CD₃OD) 1.5˜1.9 (m, 6H), 2.38 (m, 2H), 3.39 (s, 3H), 3.69 (t, 2H), 3.50 (m, 2H), 3.89 (m, 2H), 4.67 (t, 2H), 7.12 (d, 1H), 7.69 (t, 1H), 7.87 (d, 1H), 7.97 (d, 1H), 8.15 (m, 1H), 8.40 (m 1H), 8.48 (d, 1H), 8.84 (t, 1H); ESI, 488.2 [M+H].

### 3-Cyano-N-[6-(2,6-dimethyl-morpholine-4-carbonyl)-3-(3-methoxy-propyl)-3H-imidazo[4,5-b]pyridin-2-yl]-benzamide

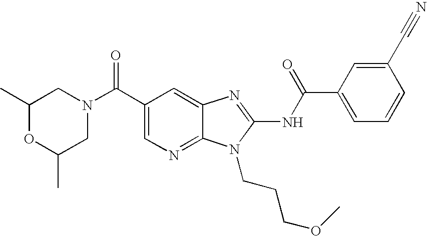

(δ_{H}, 300 MHz, CDCl₃) 1.20 (bs, 6H), 2.20 (m, 2H), 2.79 (m, 2H), 3.30 (s, 3H), 3.52 (m, 5H), 4.49 (m, 3H), 7.57 (t, 1H), 7.70 (s, 1H), 7.77 (d, 1H), 8.35 (d, 1H), 8.52 (d, 1H), 8.65 (s, 1H), ESI, 477.1 [M+H].

### 3-Cyano-N-[6-(piperidine-1-carbonyl)-3-(tetrahydro-pyran-4-ylmethyl)-3H-imidazo[4,5-b]pyridin-2-yl]-benzamide

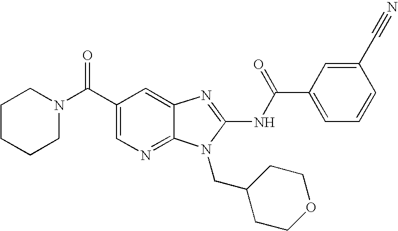

(δ_{H}, 300 MHz, CDCl₃) 1.60 (m, 10H), 2.39 (s, 1H), 3.50 (m, 6H), 4.01 (d, 2H), 4.29 (d, 2H), 7.60 (t, 1H), 7.70 (s, 1H), 7.80 (d, 1H), 8.37 (s, 1H), 8.51 (d, 1H), 8.59 (s, 1H); ESI, 473.2 [M+H].

### 3-Cyano-N-[3-(3-methoxy-propyl)-6-(piperazine-1-carbonyl)-3H-imidazo[4,5-b]pyridin-2-yl]-benzamide

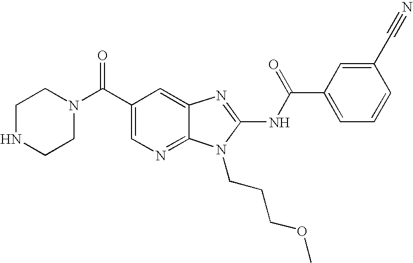

(δ_{H}, 300 MHz, d₆DMSO) 2.00 (m, 2H), 3.10 (s, 3H), 3.34 (t, 2H), 3.55 (m, 8H), 4.26 (t, 2H), 7.65 (m, 1H), 7.75 (d, 1H), 7.93 (m, 1H), 8.30 (d, 1H), 8.43 (d, 1H), 8.50 (s, 1H), 8.75 (bs, 2H); ESI, 448 [M+H].

### 2-(3-Cyano-benzoylamino)-3-(3-methoxy-propyl)-3H-imidazo[4,5-b]pyridine-6-carboxylic acid (2-hydroxy-ethyl)-methyl-amide

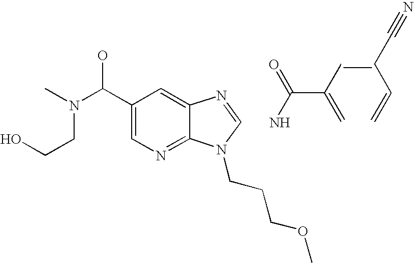

(δ_{H}, 300 MHz, d₆DMSO) 2.08 (m, 2H), 2.67 (s, 3H), 3.16 (s, 3H), 3.41 (m, 4H), 4.37 (bt, 2H), 4.55 (bt, 2H), 7.71 (t, 1H), 8.01 (d, 1H), 8.22 (s, 1H), 8.50 (d, 1H), 8.57 (s, 1H), 8.87 (bs, 1H), 8.97 (s, 1H); ESI, 437.1 [M+H].

### 2-(3-Cyanobenzamido)-3-(3-methoxypropyl)-N,N-dimethyl-3H-imidazo[4,5-b]pyridine-5-carboxamide

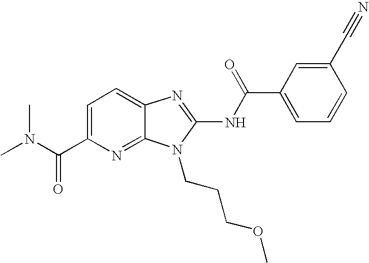

(δ_{H}, 300 MHz, CDCl₃) 2.20 (m, 2H), 3.19 (s, 6H), 3.28 (s, 3H), 3.48 (t, 2H), 4.48 (t, 2H), 7.56 (t, 1H), 7.66 (m, 2H), 7.78 (d, 1H), 8.51 (d, 1H), 8.65 (brs, 1H); ESI, 407 [M+H).

### 3-Cyano-N-[6-(2,3-dihydro-indole-1-carbonyl)-3-(3-methoxy-propyl)-3H-imidazo[4,5-b]pyridin-2-yl]-benzamide

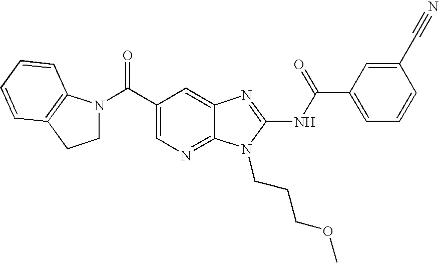

(δ_{H}, 300 MHz, CDCl₃) 2.24 (m, 2H), 3.17 (t, 2H), 3.31 (s, 3H), 3.51 (t, 2H), 4.15 (t, 2H), 4.51 (t, 2H), 7.09 (m, 4H), 7.57 (t, 1H), 7.78 (m, 2H), 8.54 (m, 2H), 8.68 (s, 1H); ESI, 481.1 [M+H].

### 3-Cyano-N-(3-ethyl-6-(piperidine-1-carbonyl)-3H-imidazo[4,5-b]pyridin-2-yl)benzamide

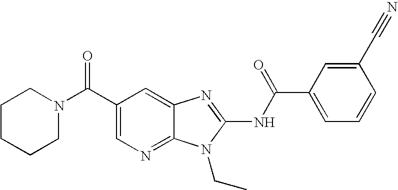

(δ_{H}, 300 MHz, CDCl₃) 1.52 (t, 3H), 1.65 (m, 6H), 3.47 (m, 4H), 4.42 (q, 2H), 7.55 (t, 1H), 7.63 (d, 1H), 7.76 (m, 1H), 8.34 (d, 1H), 8.52 (m, 1H), 8.64 (m, 1H); ESI, 403.2 [M+H].

### 3-Cyano-N-[3-[3-(2-oxo-pyrrolidin-1-yl)-propyl]-6-(piperidine-1-carbonyl)-3H-imidazo[4,5-b]pyridin-2-yl]-benzamide

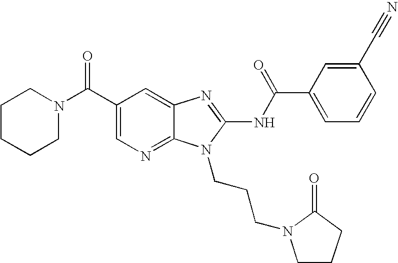

(δ_{H}, 300 MHz, CDCl₃) 1.65 (m, 6H), 2.06 (m, 2H), 2.18 (m, 2H), 2.39 (t, 2H), 3.46 (m, 6H), 3.72 (bs, 2H), 4.38 (t, 2H), 7.58 (m, 2H), 7.77 (d, 1H), 8.32 (d, 1H), 8.51 (d, 1H), 8.59 (s, 1H); ESI, 500.2 [M+H].

### N-Butyl-2-(3-cyanobenzamido)-3-(2-methoxyethyl)-3H-imidazo[4,5-b]pyridine-6-carboxamide

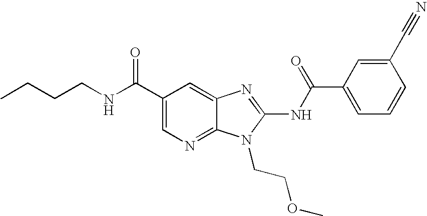

(δ_{H}, 300 MHz, CDCl₃) 0.97 (t, 3H), 1.45-1.65 (m, 4H), 3.35 (s, 3H), 3.50 (q, 2H), 3.94 (t, 2H), 4.58 (t, 2H), 6.07 (bt, 1H), 7.58 (t, 1H), 7.77 (m, 1H), 8.02 (d, 1H), 8.50 (m, 1H), 8.61 (m, 1H), 8.62 (d, 1H); ESI, 421 [M+H].

### N-[3-(3-Methoxy-propyl)-6-(piperidine-1-carbonyl)-3H-imidazo[4,5-b]pyridin-2-yl]-furan-2-carboxamide

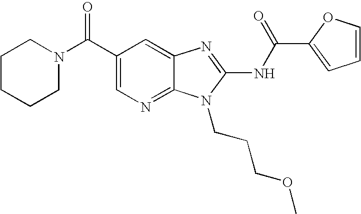

(δ_{H}, 300 MHz, CD₃OD) 1.5˜1.7 (m, 6H), 2.08 (m, 2H), 3.17 (s, 3H), 3.41 (t, 4H), 3.62 (bs, 2H), 4.37 (t, 2H), 6.83 (d, 1H), 7.50 (t, 1H), 7.73 (d, 1H), 8.19 (s, 1H), 8.25 (d, 1H); ESI, 412.4 [M+H].

### 3-Cyano-N-[6-(3-methoxy-azetidine-1-carbonyl)-3-(3-methoxy-propyl)-3H-imidazo[4,5-b]pyridin-2-yl]-benzamide

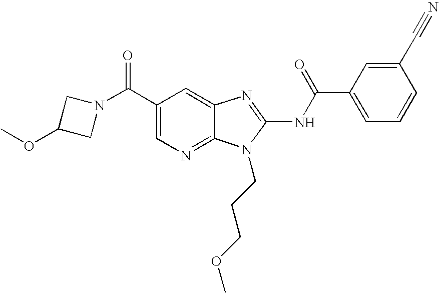

(δ_{H}, 400 MHz, CDCl₃) 2.20 (m, 2H), 3.29 (s, 3H), 3.32 (s, 3H), 3.48 (t, 2H), 4.27 (m, 3H), 4.48 (m, 4H), 7.56 (t, 1H), 7.77 (d, 1H), 7.91 (s, 1H), 8.53 (m, 2H), 8.66 (s, 1H); ESI, 449.1 [M+H].

### 2-(3-Cyano-benzamido)-3-(3-methoxypropyl)-N-(2-(methylamino)ethyl)-3H-imidazo[4,5-b]pyridin-6-carboxamide

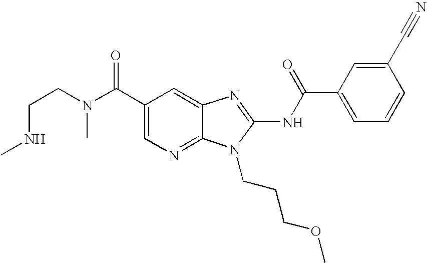

(δ_{H}, 300 MHz, CD₃OD) 2.19 (m, 2H), 2.80 (s, 3H), 3.16 (s, 3H), 3.29 (s, 3H), 3.36 (t, 2H), 3.51 (t, 2H), 3.89 (t, 2H), 4.49 (t, 2H), 7.64 (t, 1H), 7.85 (m, 1H), 7.90 (d, 1H), 8.45 (d, 1H), 8.55 (m, 1H), 8.61 (t, 1H); ESI, 450.1 [M+H].

### 2-(3-Cyano-benzamido)-3-(3-methoxypropyl)-N-methyl-N-(pyridine-4-ylmethyl)-3H-imidazo[4,5-b]pyridin-6-carboxamide

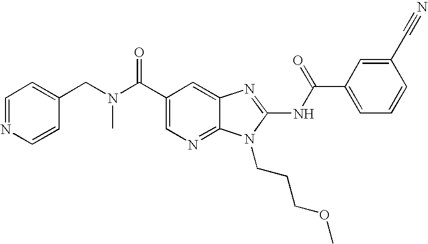

(δ_{H}, 300 MHz, CD₃OD) 2.34 (m, 2H), 3.38 (s, 3H), 3.40 (s, 3H), 3.60 (t, 2H), 4.62 (s, 2H), 5.21 (s, 2H), 7.79 (t, 1H), 8.00 (m, 1H), 8.09 (m, 1H), 8.22 (m, 2H), 8.70 (m, 2H), 8.76 (s, 1H), 8.97 (d, 1H), 9.00 (d, 1H); ESI, 484.2 [M+H].

### 3-Fluoro-N-(3-(3-methoxy-propyl)-6-piperidine-1-carbonyl)-3H-imidazo[4,5-b]pyridine-2-yl)-benzamide

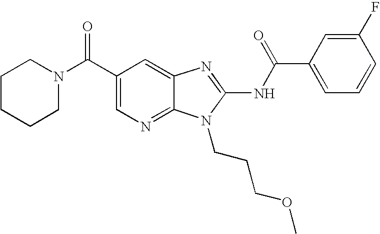

(δ_{H}, 300 MHz, CD₃OD) 1.5-1.7 (m, 6H), 2.10 (m, 2H), 3.16 (s, 3H), 3.41 (m, 2H), 3.42 (t, 2H), 3.64 (m, 2H), 4.42 (t, 2H), 7.17 (m, 1H), 7.38 (m, 1H), 7.72 (d, 1H), 7.88 (m, 1H), 8.00 (d, 1H), 8.24 (d, 1H); ESI, 440.2 [M+H].

### 3-Cyano-N-[6-(3-dimethylamino-pyrrolidine-1-carbonyl)-3-(3-methoxy-propyl)-3H-imidazo[4,5-b]pyridin-2-yl]-benzamide

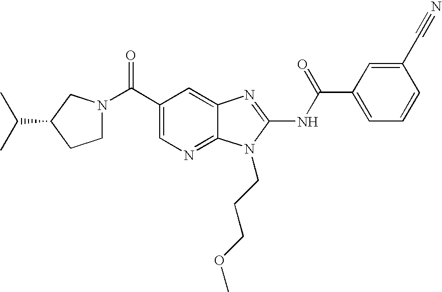

(δ_{H}, 300 MHz, CDCl₃) 2.21 (m, 2H), 2.49 (m, 2H), 2.96 (s, 6H), 3.30 (s, 3H), 3.50 (t, 2H), 3.70 (m, 2H), 3.92 (m, 1H), 4.08 (m, 2H), 4.49 (t, 2H), 7.58 (t, 1H), 7.80 (m, 2H), 8.45 (s, 1H), 8.53 (d, 1H), 8.66 (s, 1H); ESI, 476.1 [M+H].

### 2-(3-Cyano-benzoylamino)-3-(3-methoxy-propyl)-3H-imidazo[4,5-b]pyridine-6-carboxylic acid cyclopropyl-ethyl-amide

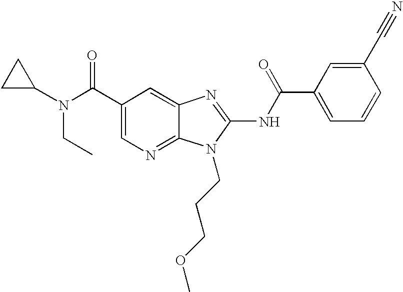

(δ_{H}, 300 MHz, d₆-DMSO) 0.44 (s, 2H), 0.60 (d, 2H), 1.20 (t, 3H), 2.09 (m, 2H), 2.92 (s, 1H), 3.17 (s, 3H), 3.45 (m, 4H), 4.36 (t, 2H), 7.72 (t, 1H), 7.85 (s, 1H), 8.02 (d, 1H), 8.44 (s, 1H), 8.52 (d, 1H), 8.59 (s, 1H); ESI, 447.2 [M+H].

### 3-Cyano-N-[6-(piperidine-1-carbonyl)-3-(2-pyrazol-1-yl-ethyl)-3H-imidazo[4,5-b]pyridin-2-yl]-benzamide

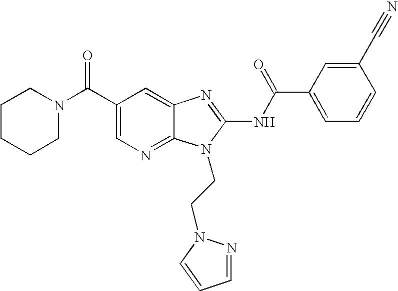

(δ_{H}, 300 MHz, CDCl₃) 1.70 (m, 6H), 3.65 (m, 4H), 4.75 (m, 4H), 6.08 (s, 1H), 7.35 (m, 2H), 7.58 (m, 2H), 7.80 (m, 2H), 8.27 (s, 1H), 8.52 (m, 2H); ESI, 469.2 [M+H].

### 3-Cyano-N-[6-(piperidine-1-carbonyl)-3-pyridin-2-ylmethyl-3H-imidazo[4,5-b]pyridin-2-yl]-benzamide

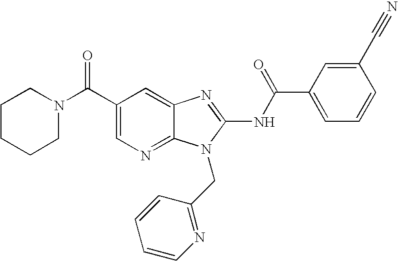

(δ_{H}, 300 MHz, CDCl₃) 1.64 (m, 6H), 3.63 (m, 4H), 5.69 (s, 2H), 7.26 (s, 1H), 7.38 (d, 1H), 7.53 (t, 1H), 7.73 (m, 3H), 8.47 (m, 4H); ESL 466.2 [M+H].

### N-(3-(3-Methoxypropyl)-6-(piperidine-1-carbonyl)-3H-imidazo[4,5-b]pyridin-2-yl)nicotinamide

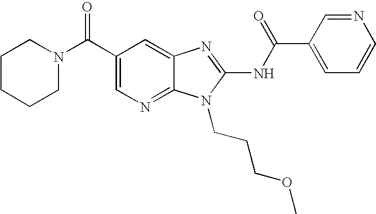

(δ_{H}, 300 MHz, CD₃OD) 1.70˜1.90 (m, 6H), 2.35 (m, 2H), 3.40 (s, 3H), 3.64 (m, 4H), 3.90 (bs, 2H), 4.68 (t, 2H), 7.95 (d, 1H), 8.32 (m, 1H), 8.51 (d, 1H), 9.12 (d, 1H), 9.48 (m, 1H), 9.73 (s, 1H); ESI 423.2 [M+H].

### 2-(3-Cyano-benzoylamino)-3-(3-methoxy-propyl)-3H-imidazo[4,5-b]pyridine-6-carboxylic acid methoxy-methyl-amide

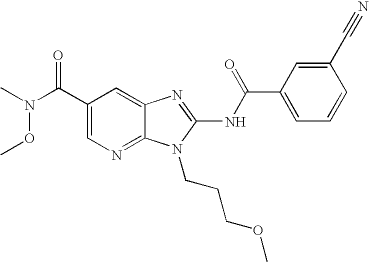

(δ_{H}, 300 MHz, CDCl₃) 2.23 (t, 2H), 3.31 (s, 3H), 3.42 (s, 3H), 3.51 (t, 2H), 3.59 (s, 3H), 4.51 (t, 2H), 7.60 (t, 1H), 7.78 (d, 1H), 7.99 (s, 1H), 8.56 (d, 1H), 8.68 (s, 1H), 8.75 (s, 1H); ESI, 423 [M+H].

### 3-Cyano-N-(6-(piperidine-1-carbonyl)-3-(2-(pyridin-2-yl)ethyl)-3H-imidazo[4,5-b]pyridin-2-yl)benzamide

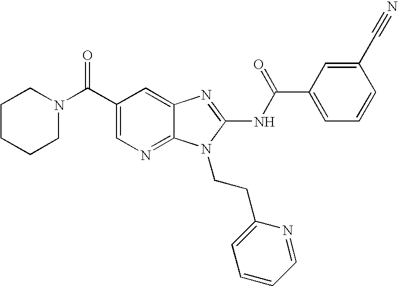

(δ_{H}, 300 MHz, CDCl₃) 1.65 (m, 6H), 3.47 (m, 6H), 4.70 (t, 2H), 6.98 (m, 1H), 7.08 (d, 1H), 7.48 (m, 2H), 7.58 (d, 1H), 7.69 (m, 1H), 8.23 (d, 1H), 8.40 (m, 2H), 8.49 (s, 1H); ESI, 480.0 [M+H].

### 2-(3-cyanobenzamido)-N,N-diethyl-3-(3-methoxypropyl)-3H-imidazo[4,5-b]pyridine-5-carboxamide

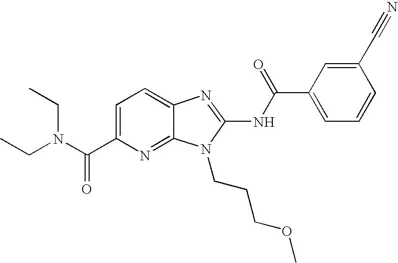

(δ_{H}, 300 MHz, CDCl₃) 1.30 (m, 6H), 2.18 (m, 2H), 3.28 (s, 3H), 3.47 (m, 4H), 3.58 (m, 2H), 4.46 (t, 2H), 7.56 (t, 1H), 7.62 (s, 2H), 7.77 (d, 1H), 8.53 (d, 1H), 8.66 (s, 1H); m/z ESI 435 [M+H].

### 3-Cyano-N-[6-(3-hydroxy-pyrrolidine-1-carbonyl)-3-(3-methoxy-propyl)-3H-imidazo[4,5-b]pyridin-2-yl]-benzamide

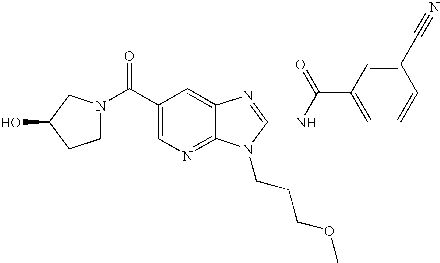

(δ_{H}, 300 MHz, CDCl₃) 2.11 (m, 4H), 3.28 (s, 3H), 3.50 (s, 2H), 3.80 (m, 4H), 4.56 (m, 3H), 7.64 (s, 1H), 7.85 (m, 2H), 8.55 (m, 3H); ESI, 449.1 [M+H].

### N-(3-(3-Methoxypropyl)-6-(piperidine-1-carbonyl)-3H-imidazo[4,5-b]pyridin-2-yl)-5-methylthiophene-2-carboxamide

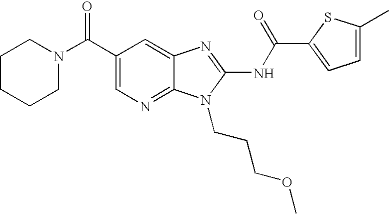

(δ_{H}, 300 MHz, CD₃OD) 1.50˜1.70 (m, 6H), 2.09 (m, 2H), 2.44 (s, 3H), 3.16 (s, 3H), 3.39 (bt, 2H), 3.42 (t, 2H), 3.64 (m, 2H), 4.34 (t, 2H), 6.74 (m, 1H), 7.60 (d, 1H), 7.68 (d, 1H), 8.21 (d, 1H); ESI 442.4 [M+H].

### N-(3-(3-Methoxypropyl)-6-(piperidine-1-carbonyl)-3H-imidazo[4,5-b]pyridin-2-yl)-3-methylbenzamide

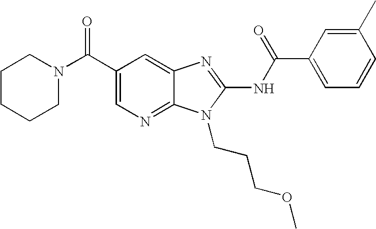

(δ_{H}, 300 MHz, CDCl₃) 1.50˜1.70 (, 6H), 2.11 (m, 2H), 2.35 (s, 3H), 3.16 (s, 3H), 3.42 (t, 4H), 3.65 (m, 2H), 4.41 (t, 2H), 7.29 (m, 2H), 7.75 (s, 1H), 7.95 (m, 1H), 7.98 (s, 1H), 8.26 (s, 1H);

ESI 436.2 [M+H].

### 3-Cyano-N-(3-(3-methoxypropyl)-5-(morpholine-4-carbonyl)-3H-imidazo[4,5-b]pyridin-2-yl)benzamide

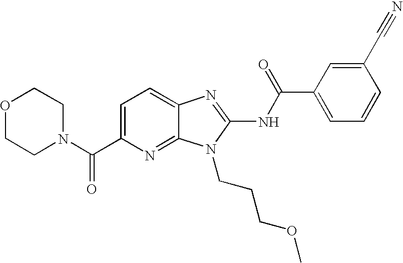

(δ_{H}, 300 MHz, d₆DMSO) 2.09 (m, 2H), 3.19 (s, 3H), 3.43 (t, 2H), 3.64 (m, 8H), 4.35 (t, 2H), 7.54 (d, 1H), 7.73 (t, 1H), 7.87 (d, 1H), 8.02 (d, 1H), 8.53 (d, 1H), 8.60 (s, 1H); ESI, 449 [M+H].

### 3-Cyano-N-[6-(4-hydroxymethyl-piperidine-1-carbonyl)-3-(3-methoxy-propyl)-3H-imidazo[4,5-b]pyridin-2-yl]-benzamide

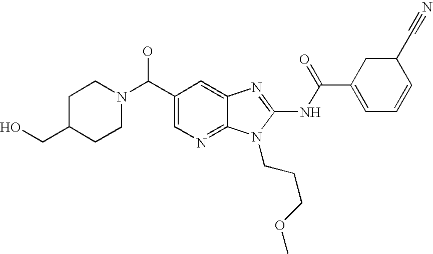

(δ_{H}, 300 MHz, CDCl₃) 1.29 (bs, 2H), 1.82 (bs, 3H), 2.21 (t, 2H), 2.98 (m, 2H), 3.34 (s, 3H), 3.54 (m, 4H), 3.94 (bs, 1H), 4.47 (t, 2H), 4.71 (bs, 1H), 7.56 (t, 1H), 7.70 (s, 1H), 7.78 (d, 1H), 8.32 (s, 1H), 8.51 (d, 1H), 8.62 (s, 1H); ESI, 477.2 [M+H].

### 2-(3-Cyanobenzamido)-N,N-diisopropyl-3-(3-methoxypropyl)-3H-imidazo[4,5-b]pyridin-6-carboxamide

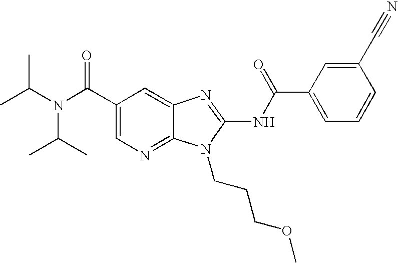

(δ_{H}, 300 MHz, CDCl₃) 1.40 (s, 12H), 2.23 (m, 2H), 3.32 (s, 3H), 3.51 (t, 2H), 3.74 (m, 2H), 4.51 (t, 2H), 7.59 (t, 2H), 7.80 (m, 1H), 8.29 (d, 1H), 8.55 (m, 1H), 8.68 (s, 1H); ESI, 463.2 [M+H].

### 3-Cyano-N-(3-(3-methoxypropyl)-6-(4-(pyrrolidin-1-yl)piperidine-1-carbonyl)-3H-imidazo[4,5-b]pyridin-2-yl)benzamide

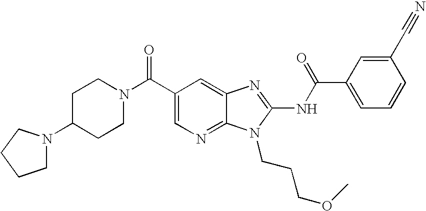

(δ_{H}, 300 MHz, CDCl₃) 1.95-2.25 (m, 10H), 3.00 (m, 4H), 3.30 (m, 4H), 3.40 (m, 2H), 3.82 (m, 2H), 4.48 (m, 4H), 7.54 (t, 1H), 7.70 (s, 1H), 7.78 (d, 1H), 8.36 (s, 1H), 8.53 (d, 1H), 8.65 (s, 1H), 12.60 (bs, 1H); ESI, 516 [M+H].

### (R)-3-Cyano-N-[3-(3-methoxy-propyl)-6-(2-methyl-pyrrolidine-1-carbonyl)-3H-imidazo[4,5-b]pyridin-2-yl]-benzamide

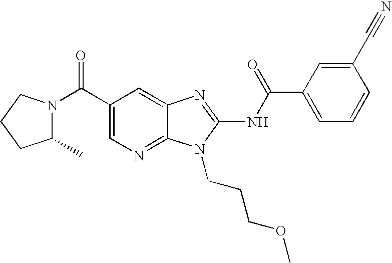

(δ_{H}, 300 MHz, CDCl₃) 1.41 (d, 3H), 1.60-2.30 (m, 6H), 3.30 (s, 3H), 3.54 (m, 4H), 4.40 (m, 1H), 4.52 (t, 2H), 7.62 (m, 1H), 7.82 (m, 2H), 8.51 (m, 2H), 8.64 (s, 1H); ESI, 447 [M+H].

### 2-(3-Cyano-benzoylamino)-3-(3-methoxy-propyl)-3H-imidazo[4,5-b]pyridine-6-carboxylic acid (3-imidazol-1-yl-propyl)-amide

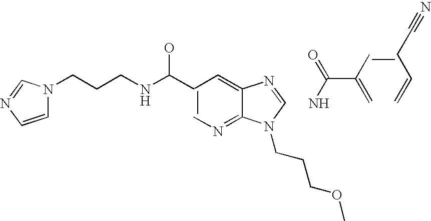

(δ_{H}, 300 MHz, d₆DMSO) 2.10 (m, 4H), 3.20 (s, 3H), 3.32 (m, 2H), 3.44 (t, 2H), 4.27 (t, 2H), 4.38 (t, 2H), 7.54 (m, 2H), 7.84 (m, 1H), 8.03 (d, 1H), 8.14 (d, 1H), 8.52 (d, 1H), 8.60 (s, 1H), 8.73 (d, 1H), 8.77 (m, 1H), 9.14 (s, 1H); ESI, 487 [M+H].

### 3-Cyano-N-(3-(3-methoxypropyl)-6-(4-(2-methylpiperidin-1-yl)piperidine-1-carbonyl)-3H-imidazo[4,5-b]pyridin-2-yl)benzamide

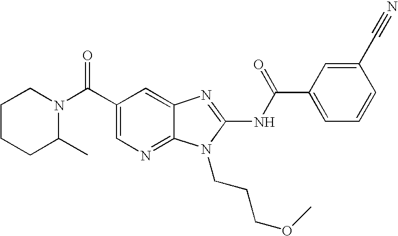

(δ_{H}, 400 MHz, CDCl₃) 1.30 (d, 3H), 1.40-1.78 (m, 7H), 2.21 (m, 2H), 3.10 (m, 1H), 3.30 (s, 3H), 3.48 (m, 2H), 4.49 (m, 3H), 7.58 (t, 1H), 7.66 (s, 1H), 7.79 (d, 1H), 8.34 (s, 1H), 8.53 (d, 1H), 8.67 (s, 1H); ESI, 461 [M+H].

### 3-Cyano-N-(6-(piperidine-1-carbonyl)-3-(tetrahydro-2H-pyran-4-yl)-3H-imidazo[4,5-b]pyridin-2-yl)benzamide

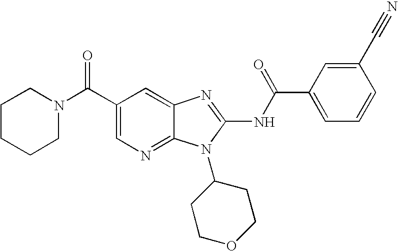

(δ_{H}, 300 MHz, CDCl₃) 1.65 (m, 6H), 1.82 (dd, 2H), 3.03 (m, 2H), 3.53 (m, 6H), 4.19 (dd, 2H), 5.13 (m, 1H), 7.57 (t, 1H), 7.64 (d, 1H), 7.77 (m, 1H), 8.31 (d, 1H), 8.52 (m, 1H), 8.58 (s, 1H); ESI, 459.1 [M+H].

### 3-Cyano-N-[3-(4-methoxy-benzyl)-6-(piperidine-1-carbonyl)-3H-imidazo[4,5-b]pyridin-2-yl]-benzamide

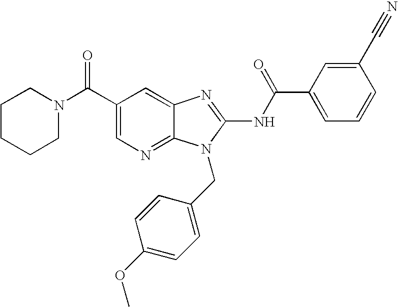

(δ_{H}, 300 MHz, CDCl₃) 1.67 (m, 6H), 3.61 (m, 4H), 3.72 (s, 3H), 5.50 (s, 2H), 6.82 (d, 1H), 7.15 (d, 2H), 7.26 (m, 1H), 7.59 (m, 2H), 7.79 (d, 1H), 8.36 (s, 1H), 8.54 (d, 1H), 8.65 (s, 1H); ESI, 495.1 [M+H].

### 3-Cyano-N-(6-(piperidine-1-carbonyl)-3-(pyridin-3-ylmethyl)-3H-imidazo[4,5-b]pyridin-2-yl)benzamide

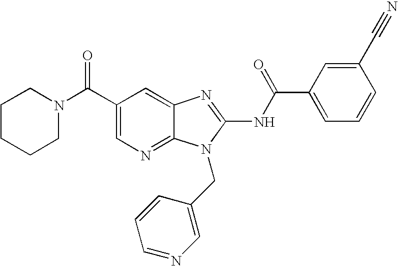

(δ_{H}, 300 MHz, CDCl₃) 1.69 (m, 6H), 3.50 (m, 4H), 5.53 (s, 2H), 7.25 (m, 1H), 7.61 (m, 2H), 7.78 (d, 2H), 7.91 (d, 1H), 8.34 (s, 1H), 8.52 (m, 2H), 8.60 (s, 1H), 8.89 (s, 1H); ESI, 466.2 [M+H].

### N-tert-Butyl-2-(3-cyanobenzamido)-3-(3-methoxypropyl)-N-methyl-3H-imidazo[4,5-b]pyridine-6-carboxamide

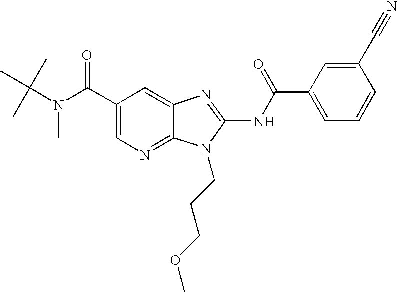

(δ_{H}, 300 MHz, CDCl₃) 1.47 (s, 9H), 2.18 (m, 2H), 2.89 (s, 3H), 3.25 (s, 3H), 3.45 (t, 2H), 4.45 (t, 2H) 7.53 (t, 1H), 7.61 (d, 1H), 7.73 (d, 1H), 8.34 (d, 1H), 8.52 (d, 1H), 8.64 (s, 1H), ESI, 449.2 [M+H].

### (S)-3-Cyano-N-(6-(2-(hydroxymethyl)pyrrolidine-1-carbonyl)-3-(3-methoxypropyl)-3H-imidazo[4,5-b]pyridin-2-yl)benzamide

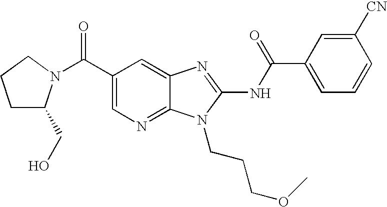

(δ_{H}, 400 MHz, CDCl₃) 1.80 (m, 2H), 1.95 (m, 1H), 2.18 (m, 3H), 3.28 (s, 3H), 3.48 (m, 2H), 3.58 (m, 2H), 3.93 (m, 2H), 4.45 (m, 3H), 7.54 (m, 1H), 7.75 (m, 1H), 7.92 (m, 1H), 8.47 (m, 2H), 8.60 (m, 1H); ESI, 463 [M+H].

### 3-Cyano-N-(3-methyl-6-(piperidine-1-carbonyl)-3H-imidazo[4,5-b]pyridin-2-yl)benzamide

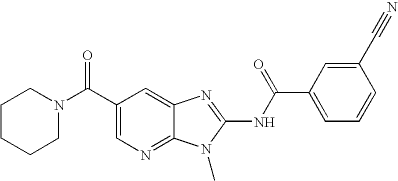

(δ_{H}, 300 MHz, CDCl₃) 1.69 (m, 6H), 3.59 (m, 4H), 3.83 (s, 3H), 7.56 (t, 1H), 7.64 (d, 1H), 7.76 (m, 1H), 8.34 (d, 1H), 8.52 (m, 1H), 8.65 (t, 1H); ESI, 389.3 [M+H].

### N-(3-(2-Aminoethyl)-6-(piperidine-1-carbonyl)-3H-imidazo[4,5-b]pyridin-2-yl)-3-cyanobenzamide

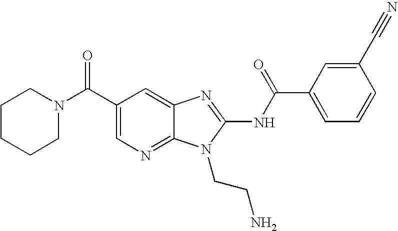

(δ_{H}, 300 MHz, CD₃OD) 1.74 (m, 6H), 3.48 (s, 2H), 3.58 (t, 2H), 3.74 (s, 2H), 4.71 (t, 2H), 7.64 (t, 1H), 7.81 (d, 1H), 7.86 (dd, 1H), 8.32 (d, 1H), 8.54 (dd, 1H), 8.62 (s, 1H); ESI, 418.1 [M+H].

### 3-Cyano-N-(6-(piperidine-1-carbonyl)-3-(2-(tetrahydrofuran-2-yl)ethyl)-3H-imidazo[4,5-b]pyridin-2-yl)benzamide

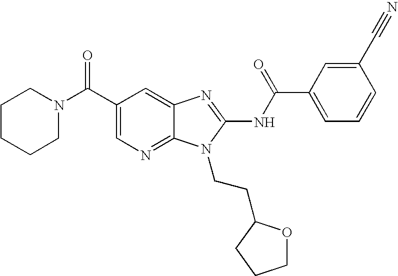

(δ_{H}, 400 MHz, CDCl₃) 1.50-1.75 (m, 8H), 1.90 (m, 2H), 2.12 (m, 2H), 3.43 (m, 4H), 3.56 (m, 2H), 3.95 (m, 1H), 4.51 (m, 2H), 7.58 (t, 1H), 7.68 (s, 1H), 7.79 (d, 1H), 8.47 (s, 1H), 8.52 (d, 1H), 8.68 (s, 1H); ESL 473 [M+H].

### 3-Cyano-N-(6-(3-hydroxyazetidine-1-carbonyl)-3-(3-methoxypropyl)-3H-imidazo[4,5-b]pyridin-2-yl)benzamide

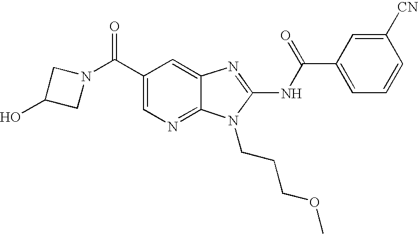

(δ_{H}, 300 MHz, CDCl₃) 2.21 (m, 2H), 3.29 (s, 3H), 3.51 (m, 2H), 4.18 (m, 2H), 4.49 (m, 4H), 4.77 (s, 1H), 7.57 (t, 1H), 7.78 (d, 1H), 7.97 (s, 1H), 8.54 (m, 3H); ESI, 435 [M+H].

### 3-Cyano-N-[3-(3-methoxy-benzyl)-6-(piperidine-1-carbonyl)-3H-imidazo[4,5-b]pyridin-2-yl]-benzamide

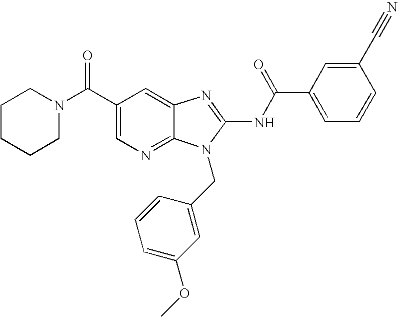

(δ_{H}, 300 MHz, CDCl₃) 1.83 (m, 6H), 3.51 (m, 4H), 3.76 (s, 3H), 5.50 (s, 2H), 6.81 (d, 1H), 7.14 (d, 2H), 7.25 (m, 1H), 7.58 (m, 2H), 7.78 (d, 1H), 8.36 (s, 1H), 8.54 (d, 1H), 8.65 (s, 1H); ESI, 495.2 [M+H].

### 3-Cyano-N-(3-(3-methoxypropyl)-6-(4-(piperidin-1-yl)piperidine-1-carbonyl)-3H-imidazo[4,5-b]pyridin-2-yl)benzamide

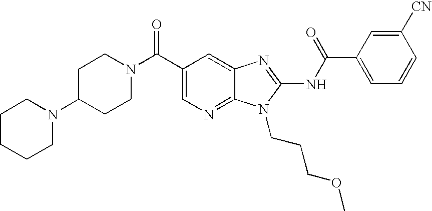

(δ_{H}, 300 MHz, CDCl₃) 1.41 (m, 1H), 1.75 (m, 2H), 1.85 (m, 5H), 2.22 (m, 4H), 2.78 (m, 2H), 3.06 (m, 2H), 3.30 (s, 3H), 3.56 (m, 5H), 4.52 (m, 4H), 7.59 (m, 1H), 7.79 (m, 2H), 8.40 (s, 1H), 8.49 (m, 1H), 8.62 (m, 1H); ESI, 530 [M+H].

### 2-(3-Cyanobenzamido)-N-ethyl-N-isopropyl-3-(3-methoxypropyl)-3H-imidazo[4,5-b]pyridin-6-carboxamide

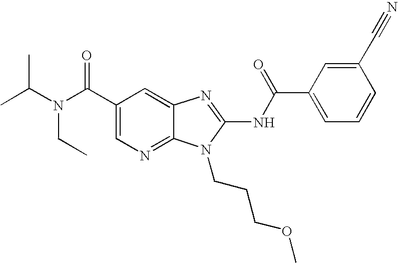

(δ_{H}, 300 MHz, CDCl₃) 1.40 (s, 9H), 2.23 (m, 2H), 3.32 (s, 3H), 3.43 (m, 2H), 3.51 (t, 2H), 4.06 (m, 1H), 4.50 (t, 2H), 7.59 (t, 2H), 7.80 (m, 1H), 8.29 (d, 1H), 8.55 (m, 1H), 8.68 (s, 1H); ESI, 449.2 [M+H].

### N-[6-(4-Benzyl-piperazine-1-carbonyl)-3-(3-methoxy-propyl)-3H-imidazo[4,5-b]pyridin-2-yl]-3-cyano-benzamide

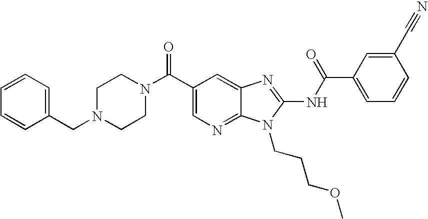

(δ_{H}, 300 MHz, d₆DMSO) 2.00 (m, 2H), 3.10 (s, 3H), 3.35 (t, 2H), 3.55 (m, 8H), 4.30 (m, 4H), 7.40 (m, 5H), 7.62 (m, 1H), 7.73 (d, 1H), 7.92 (m, 1H), 8.28 (d, 1H), 8.45 (d, 1H), 8.52 (s, 1H); ESI, 538 [M+H].

### 3-Cyano-N-[3-(2-hydroxy-ethyl)-6-(piperidine-1-carbonyl)-3H-imidazo[4,5-b]pyridin-2-yl]-benzamide

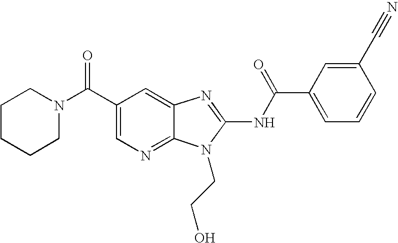

(δ_{H}, 300 MHz, CDCl₃) 1.67 (r, 6H), 3.63 (m, 4H), 4.08 (m, 3H), 4.63 (m, 2H), 7.59 (t, 1H), 7.70 (s, 1H), 7.80 (d, 1H), 8.35 (s, 1H), 8.49 (d, 1H), 8.59 (s, 1H); ESI, 419.2 [M+H].

### 2-(3-Cyanobenzamido)-N-isopropyl-3-(3-methoxypropyl)-N-methyl-3H-imidazo[4,5-b]pyridin-6-carboxamide

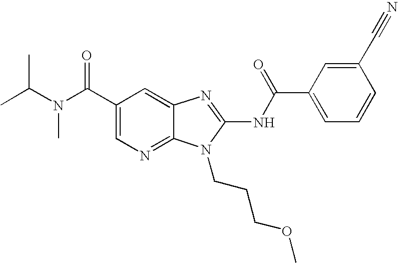

(δ_{H}, 300 MHz, CDCl₃) 1.25 (d, 6H), 2.23 (m, 2H), 3.00 (s, 3H), 3.31 (s, 3H), 3.52 (t, 2H), 4.06 (m, 1H), 4.52 (t, 2H), 7.59 (t, 1H), 7.69 (s, 1H), 7.80 (m, 1H), 8.37 (d, 1H), 8.53 (m, 1H), 8.67 (s, 1H); ESI, 435.2 [M+H].

### Methyl 3-(2-(3-cyanobenzamido)-6-(piperidine-1-carbonyl)-3H-imidazo[4,5-b]pyridin-3-yl)propanoate

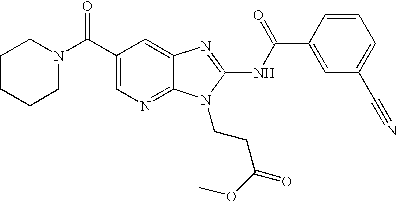

(δ_{H}, 400 MHz, CDCl₃) 1.55-1.85 (m, 6H), 3.10 (m, 2H), 3.40-3.85 (m, 7H), 4.75 (m, 2H), 7.65 (t, 1H), 7.72 (s, 1H), 7.86 (d, 1H), 8.42 (s, 1H), 8.60 (d, 1H), 8.69 (s, 1H); ESI, 461 [M+H].

### 3-Cyano-N-[3-(3-hydroxy-propyl)-6-(piperidine-1-carbonyl)-3H-imidazo[4,5-b]pyridin-2-yl]-benzamide

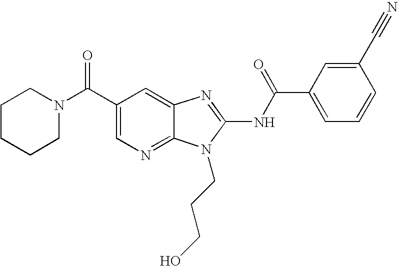

(δ_{H}, 300 MHz, CDCl₃) 1.65 (m, 6H), 2.15 (s, 2H), 3.45 (m, 6H), 4.10 (s, 1H), 4.57 (t, 1H), 7.59 (t, 1H), 7.71 (s, 1H), 7.80 (d, 1H), 8.35 (s, 1H), 8.49 (d, 1H), 8.61 (s, 1H); ESI, 433.2 [M+H].

### (+/−)-2-(3-Cyano-benzoylamino)-3-(3-methoxy-propyl)-3H-imidazo[4,5-b]pyridine-6-carboxylic acid (1-ethyl-pyrrolidin-2-ylmethyl)-amide

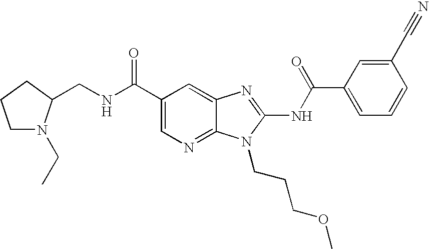

(δ_{H}, 300 MHz, d₆DMSO) 1.28 (m, 3H), 2.00 (m, 6H), 3.20 (m, 2H), 3.40 (s, 3H), 3.45 (t, 2H), 3.50 (m, 1H), 3.65 (m, 4H), 4.40 (t, 2H), 7.75 (m, 1H), 8.05 (d, 1H), 8.16 (d, 1H), 8.50 (d, 1H), 8.60 (s, 1H), 8.75 (d, 1H), 9.05 (bt, 1H), 9.22 (bs, 1H); ESL 490 [M+H].

### 3-Cyano-N-(3-(2-cyanoethyl)-6-(piperidine-1-carbonyl)-3H-imidazo[4,5-b]pyridin-2-yl)benzamide

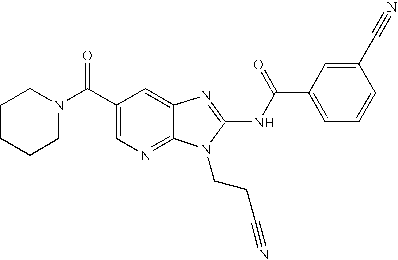

(δ_{H}, 300 MHz, CDCl₃) 1.65 (m, 6H), 3.13 (t, 2H), 3.56 (m, 4H), 4.67 (t, 2H), 7.57 (t, 1H), 7.66 (d, 1H), 7.78 (m, 1H), 8.33 (d, 1H), 8.50 (m, 1H), 8.59 (t, 1H); ESI, 428.1 [M+H].

### N-(5-(Azetidine-1-carbonyl)-3-(3-methoxypropyl)-3H-imidazo[4,5-b]pyridin-2-yl)-3-cyanobenzamide

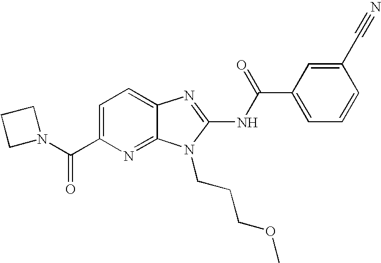

(δ_{H}, 300 MHz, CDCl₃) 2.20 (m, 2H), 2.40 (m, 2H), 3.28 (s, 3H), 3.46 (t, 2H), 4.29 (t, 2H), 4.45 (t, 2H), 4.86 (t, 2H), 7.56 (t, 1H), 7.64 (d, 1H), 7.77 (d, 1H), 8.12 (d, 1H), 8.51 (d, 2H), 8.66 (s, 1H); ESI, 419 [M+H].

### 3-Cyano-N-(3-(3-methoxypropyl)-6-(4-phenylpiperidine-1-carbonyl)-3H-imidazo[4,5-b]pyridin-2-yl)benzamide

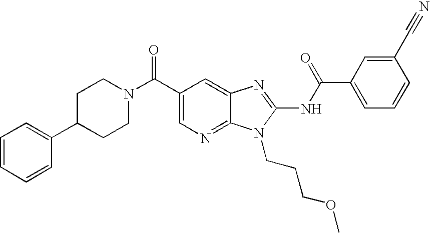

(δ_{H}, 300 MHz, CDCl₃) 1.60-2.10 (m, 4H), 2.22 (m, 2H), 2.84 (m, 2H), 3.30 (m, 4H), 3.51 (m, 2H), 3.97 (m, 1H), 4.51 (m, 3H), 7.23 (m, 3H), 7.33 (m, 2H), 7.57 (m, 1H), 7.78 (m, 2H), 8.43 (s, 1H), 8.53 (d, 1H), 8.65 (s, 1H); ESI, 523 [M+H].

### (S)-3-Cyano-N-(3-(3-methoxypropyl)-6-(3-methylmorpholine-4-carbonyl)-3H-imidazo[4,5-b]pyridin-2-yl)benzamide

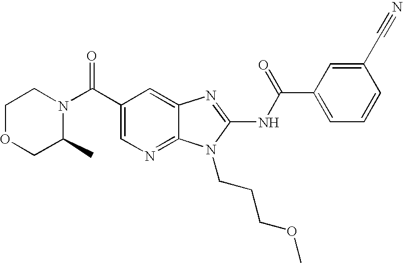

(δ_{H}, 400 MHz, CDCl₃) 1.40 (d, 3H), 2.20 (m, 2H), 3.29 (s, 3H), 3.49 (m, 4H), 3.67 (m, 3H), 3.92 (d, 1H), 4.48 (t, 2H), 7.56 (m, 1H), 7.70 (s, 1H), 7.77 (d, 1H), 8.34 (s, 1H), 8.51 (d, 1H), 8.64 (s, 1H), 10.05 (bs, 1H); ESI, 463 [M+H].

### 3-Cyano-N-(3-cyclopropyl-6-(piperidine-1-carbonyl)-3H-imidazo[4,5-b]pyridin-2-yl)benzamide

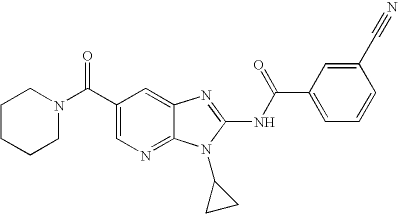

(δ_{H}, 300 MHz, CDCl₃) 1.36 (m, 2H), 1.42 (m, 2H), 1.62 (m, 6H), 3.51 (m, 5H), 7.55 (t, 1H), 7.62 (d, 1H), 7.75 (m, 1H), 8.33 (d, 1H), 8.52 (d, 1H), 8.63 (s, 1H); ESI, 415.2 [M+H].

### 3-Cyano-N-(6-(isoindoline-2-carbonyl)-3-(3-methoxypropyl)-3H-imidazo[4,5-b]pyridin-2-yl)benzamide

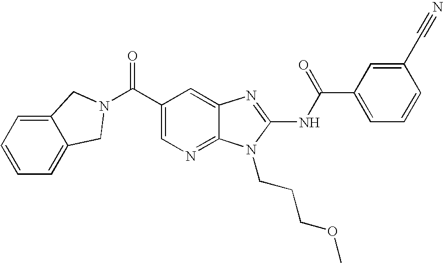

(δ_{H}, 300 MHz, CDCl₃) 2.25 (m, 2H), 3.32 (s, 3H), 3.52 (t, 2H), 4.52 (t, 2H), 4.88 (s, 2H), 5.08 (s, 2H), 7.18 (d, 1H), 7.34 (m, 3H), 7.58 (m, 1H), 7.80 (m, 2H), 8.55 (m, 2H), 8.69 (s, 1H); ESI, 481 [M+H].

### (+/−)-3-Cyano-N-[6-(2-ethyl-piperidine-1-carbonyl)-3-(3-methoxy-propyl)-3H-imidazo[4,5-b]pyridin-2-yl]-benzamide

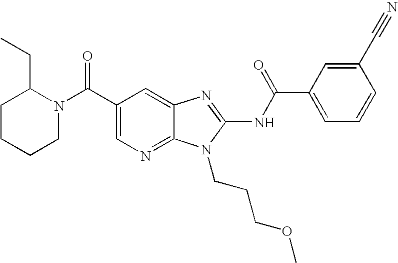

(δ_{H}, 300 MHz, CDCl₃) 0.90 (m, 3H), 1.40-1.90 (m, 10H), 2.23 (m, 3H), 3.31 (s, 3H), 3.51 (t, 2H), 4.52 (t, 2H), 7.59 (t, 1H), 7.68 (s, 1H), 7.81 (d, 1H), 8.35 (s, 1H), 8.52 (d, 1H), 8.66 (s, 1H); ESI, 475 [M+H].

### N-(6-(Azepane-1-carbonyl)-3-(3-methoxypropyl)-3H-imidazo[4,5-b]pyridin-2-yl)-3-cyanobenzamide

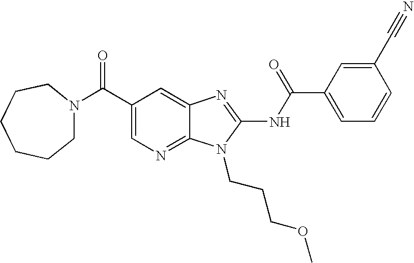

(δ_{H}, 300 Hz, CDCl₃) 1.65 (m, 6H), 1.88 (m, 2H), 2.22 (m, 2H), 3.30 (s, 3H), 3.50 (m, 4H), 3.72 (t, 2H), 4.50 (t, 2H), 7.58 (m, 1H), 7.66 (s, 1H), 7.79 (d, 1H), 8.36 (d, 1H), 8.53 (d, 1H), 8.67 (s, 1H); ESI, 461 [M+H].

### N-(3-(2-(1H-Imidazol-5-yl)ethyl)-6-(piperidine-1-carbonyl)-3H-imidazo[4,5b]pyridin-2-yl)-3-cyanobenzamide

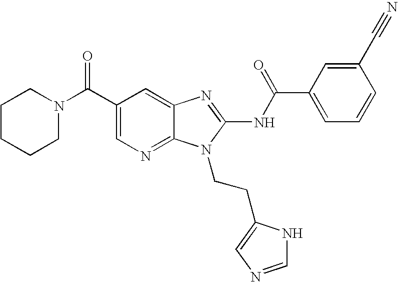

(δ_{H}, 400 MHz, CDCl₃) 1.65 (m, 6H), 3.35 (m, 4H), 3.68 (m, 2H), 4.65 (m, 2H), 7.05 (s, (m, 2H), 7.70 (d, 1H), 8.10 (s, 1H), 8.21 (s, 1H), 8.45 (m, 2H), 14.40 (bs, 1H); [M+H].

### 2-(3-Cyanobenzamido)-N,N-diethyl-3-(3-methoxypropyl)-3H-imidazo[4,5b]pyridine-6-carboxamide

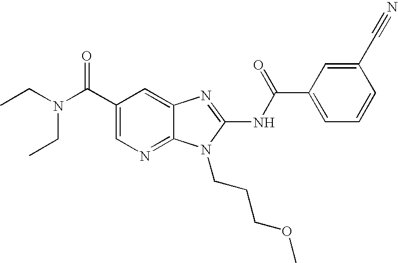

(δ_{H}, 300 MHz, CDCl₃) 1.24 (m, 6H), 2.22 (m, 2H), 3.30 (s, 3H), 3.49 (m, 4H), 3.51 (t, 2H), 4.49 (t, 2H), 7.60 (m, 2H), 7.78 (m, 1H), 8.34 (d, 1H), 8.55 (d, 1H), 8.67 (s, 1H); ESI, 435 [M+H].

### 2-(3-Cyanobenzamido)-3-(3-methoxypropyl)-N-methyl-N-propyl-3H-imidazo[4,5-b]pyridine-6-carboxamide

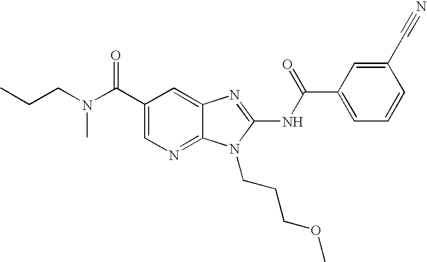

(δ_{H}, 400 MHz, CDCl₃) 0.70-1.05 (m, 3H), 1.68 (m, 2H), 2.23 (m, 2H), 3.07 (m, 3H), 3.29 (m, 4H), 3.50 (m, 3H), 4.48 (t, 2H), 7.57 (m, 1H), 7.62 (m, 1H), 7.77 (d, 1H), 8.35 (s, 1H), 8.54 (d, 1H), 8.67 (s, 1H); ESI, 435 [M+H].

### 3-Cyano-N-(3-(3-methoxypropyl)-6-(morpholine-4-carbonyl)-3H-imidazo[4,5-b]pyridin-2-yl)benzamide

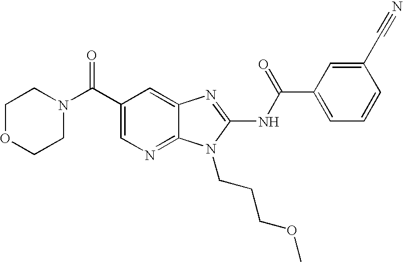

(δ_{H}, 300 MHz, CDCl₃) 2.19 (m, 2H), 3.28 (s, 3H), 3.48 (t, 2H), 3.70 (bs, 8H), 4.46 (t, 2H), 7.54 (t, 1H), 7.67 (d, 1H), 7.76 (m, 1H), 8.33 (d, 1H), 8.51 (m, 1H), 8.65 (s, 1H); ESI, 449.1 [M+H].

### 3-Cyano-N-[3-(3-methoxy-propyl)-6-(2-propyl-pyrrolidine-1-carbonyl)-3H-imidazo[4,5-b]pyridin-2-yl]-benzamide

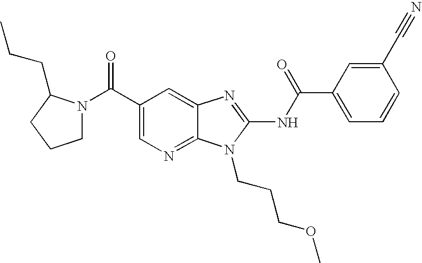

(δ_{H}, 300 MHz, CDCl₃) 0.99 (m, 3H), 1.44 (m, 3H), 1.77 (m, 2H), 2.01 (m, 2H), 2.22 (m, 3H), 3.30 (s, 3H), 3.52 (m, 4H), 4.32 (m, 1H), 4.51 (m, 2H), 7.59 (t, 1H), 7.79 (m, 2H), 8.50 (m, 2H), 8.65 (s, 1H); ESI, 475 [M+H].

### 2-(3-Cyanobenzamido)-N-cyclohexyl-3-(3-methoxypropyl)-3H-imidazo[4,5-b]pyridine-6-carboxamide

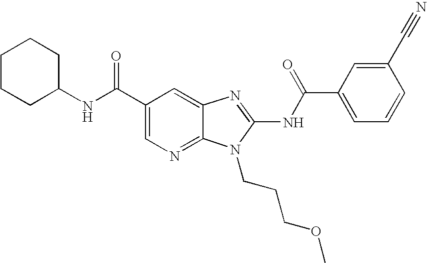

(δ_{H}, 400 MHz, CDCl₃) 1.23 (m, 4H), 1.42 (m, 2H), 1.79 (m, 2H), 2.05 (m, 2H), 2.20 (m, 2H), 3.28 (s, 3H), 3.49 (t, 2H), 4.00 (m, 1H), 4.48 (t, 2H), 6.07 (d, 1H), 7.56 (m, 1H), 7.77 (d, 1H), 8.02 (s, 1H), 8.52 (d, 1H), 8.64 (m, 2H); ESI, 461 [M+H].

### 3-Cyano-N-[3-(3-methoxy-propyl)-6-(pyrrolidine-1-carbonyl)-3H-imidazo[4,5-b]pyridin-2-yl]-benzamide

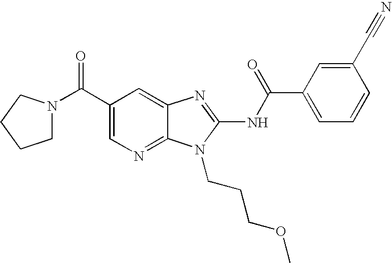

(δ_{H}, 300 MHz, d₆DMSO) 1.75 (m, 4H), 2.00 (m, 2H), 3.20 (s, 3H), 3.33 (t, 2H), 3.40 (m, 4H), 4.28 (t, 2H), 7.63 (m, 1H), 7.80 (d, 1H), 7.92 (d, 1H), 8.35 (d, 1H), 8.42 (d, 1H), 8.50 (s, 1H); ESI, 433 [M+H].

### N-(6-(Azetidine-1-carbonyl)-3-(3-methoxypropyl)-3H-imidazo[4,5-b]pyridin-2-yl)-3-cyanobenzamide

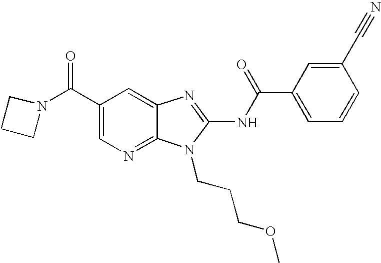

(δ_{H}, 300 MHz, CDCl₃) 2.20 (m, 2H), 2.39 (m, 2H), 3.29 (s, 3H), 3.49 (t, 2H), 4.30 (t, 2H), 4.40 (t, 2H), 4.47 (t, 2H), 7.55 (m, 1H), 7.75 (d, 1H), 7.93 (d, 1H), 8.53 (m, 2H), 8.64 (m, 2H); ESI, 419 [M+H].

### 2-(3-Cyano-benzoylamino)-3-(3-methoxy-propyl)-3H-imidazo[4,5-b]pyridine-6-carboxylic acid cyclopropylamide

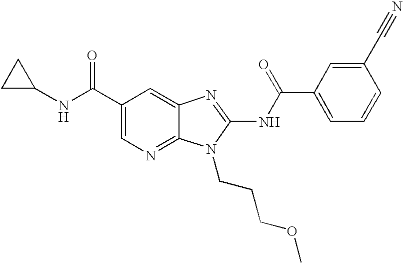

(δ_{H}, 300 MHz, CD₃OD) 0.58 (m, 2H), 0.78 (m, 2H), 2.10 (m, 2H), 2.80 (m, 1H), 3.18 (s, 3H), 3.43 (t, 2H), 4.42 (t, 2H), 7.59 (m, 1H), 7.78 (m, 1H), 8.05 (m, 1H), 8.48 (dd, 1H), 8.56 (s, 1H), 8.62 (d, 1H); ESI, 419 [M+H].

### (R)-3-Cyano-N-(3-(3-methoxypropyl)-6-(3-methylmorpholine-4-carbonyl)-3H-imidazo[4,5-b]pyridin-2-yl)benzamide

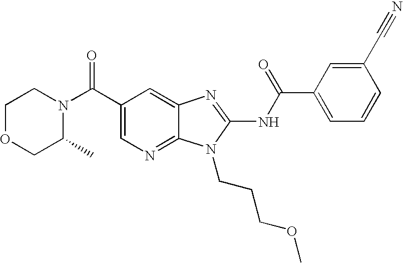

(δ_{H}, 400 MHz, CDCl₃) 1.40 (d, 3H), 2.22 (m, 2H), 3.30 (s, 3H), 3.49 (m, 4H), 3.67 (m, 3H), 3.92 (d, 2H), 4.48 (t, 2H), 7.56 (m, 1H), 7.70 (s, 1H), 7.78 (d, 1H), 8.34 (s, 1H), 8.53 (d, 1H), 8.67 (s, 1H); ESI, 463 [M+H].

### 2-(3-Cyanobenzamido)-N-ethyl-3-(3-methoxypropyl)-N-(pyridin-4-ylmethyl)-3H-imidazo[4,5-b]pyridine-6-carboxamide

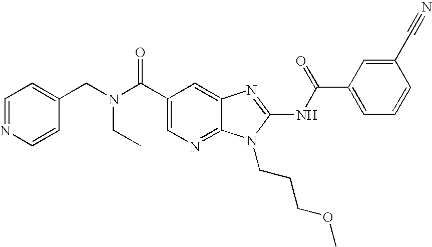

(δ_{H}, 400 MHz, CDCl₃) 1.24 (t, 3H), 2.19 (m, 2H), 3.28 (s, 3H), 3.50 (m, 4H), 4.48 (t, 2H), 4.90 (s, 2H), 7.54 (m, 1H), 7.75 (m, 4H), 8.39 (s, 1H), 8.50 (d, 1H), 8.52 (s, 1H), 8.85 (d, 2H); ESI, 498 [M+H].

### 3-Cyano-N-[3-(3-methoxy-propyl)-6-(4-methyl-piperazine-1-carbonyl)-3H-imidazo[4,5-b]pyridin-2-yl]-benzamide

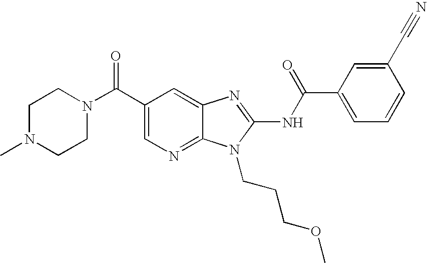

(δ_{H}, 300 MHz, d₆DMSO) 2.00 (m, 2H), 2.75 (s, 3H), 3.10 (s, 3H), 3.35 (t, 2H), 3.55 (m, 8H), 4.30 (t, 2H), 7.62 (m, 1H), 7.73 (d, 1H), 7.86 (m, 1H), 8.37 (d, 1H), 8.45 (d, 1H), 8.52 (s, 1H); ESI, 462 [M+H].

### N-(2-(1H-Imidazol-1-yl)ethyl)-2-(3-cyanobenzamido)-3-(3-methoxypropyl)-N-methyl-3H-imidazo[4,5-b]pyridine-6-carboxamide

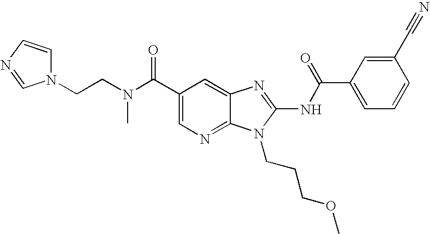

(δ_{H}, 300 MHz, CDCl₃) 2.17 (m, 2H), 3.12 (s, 3H), 3.26 (s, 3H), 3.46 (t, 2H), 4.08 (b, 2H), 4.41 (t, 2H), 4.61 (b, 1H), 7.38 (s, 1H), 7.50 (m, 2H), 7.74 (m, 2H), 8.27 (s, 1H), 8.46 (d, 2H), 8.53 (s, 1H), 9.29 (s, 1H); ESI, 487 [M+H].

### 2-(3-Cyano-benzoylamino)-3-(3-methoxy-propyl)-3H-imidazo[4,5-b]pyridine-6-carboxylic acid (2-morpholin-4-yl-ethyl)-amide

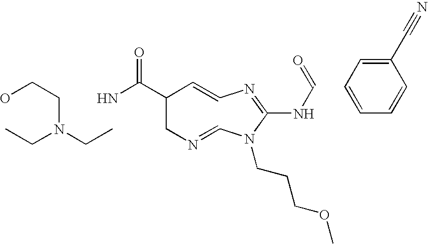

(δ_{H}, 300 MHz, d₆DMSO) 2.00 (m, 2H), 3.05 (s, 3H), 3.20-3.70 (m, 12H), 3.90 (m, 2H), 4.30 (m, 2H), 7.73 (d, 1H), 7.92 (m, 1H), 8.18 (d, 1H), 8.45 (d, 1H), 8.50 (s, 1H), 8.53 (d, 1H), 8.56 (bt, 1H), 9.60 (bs, 1H); ESI, 492 [M+H].

### 2-(3-Cyano-benzoylamino)-3-(3-methoxy-propyl)-3H-imidazo[4,5-b]pyridine-6-carboxylic acid

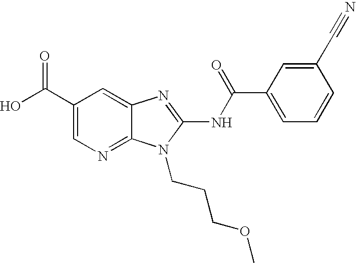

(δ_{H}, 300 MHz, d⁶DMSO) 2.00 (m, 2H), 3.08 (s, 3H), 3.35 (t, 2H), 4.28 (t, 2H), 7.60 (m, 1H), 7.90 (d, 1H), 8.10 (d, 1H), 8.43 (d, 1H), 8.50 (s, 1H), 8.71 (d, 1H), 12.8 (bs, 1H), 13.1 (bs, 1H); m/z ESI 380.3 [M+H].

### N-(3-(3-Acetamidopropyl)-6-(piperidine-1-carbonyl)-3H-imidazo[4,5-b]pyridin-2-yl)-3-cyanobenzamide

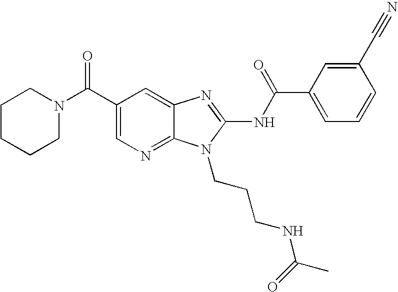

(δ_{H}, 300 MHz, CDCl₃) 1.64 (m, 6H), 2.02 (s, 3H), 2.15 (d, 2H), 3.24 (d, 2H), 3.42 (s, 2H), 3.73 (s, 2H), 4.42 (s, 2H), 7.18 (s, 1H), 7.55 (t, 1H), 7.76 (m, 1H), 8.34 (s, 1H), 8.44 (m, 1H), 8.52 (s, 1H); ESI, 474.1 [M+H].

### Ethyl 2-(3-cyanobenzamido)-3-(3-methoxypropyl)-3H-imidazo[4,5-b]pyridine-5-carboxylate

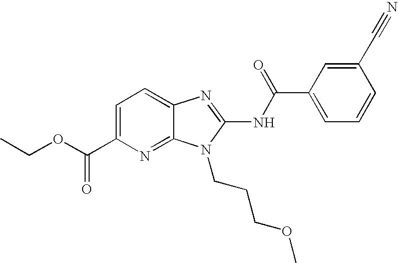

(δ_{H}, 300 MHz, CDCl₃) 1.43 (t, 3H), 2.23 (m, 2H), 3.28 (s, 3H), 3.50 (t, 2H), 4.46 (q, 2H), 4.55 (t, 2H), 7.55 (t, 1H), 7.62 (d, 1H), 7.88 (d, 1H), 8.08 (d, 1H), 8.54 (d, 1H), 8.66 (s, 1H); ESI, 408 [M+H].

### N-(3-(1-Acetylazetidin-3-yl)-6-(piperidine-1-carbonyl)-3H-imidazo[4,5-b]pyridin-2-yl)-3-cyanobenzamide

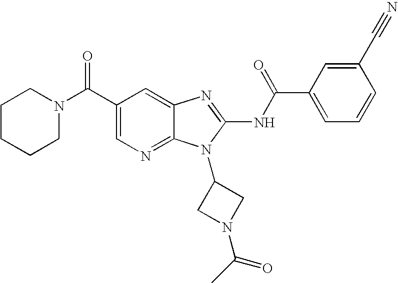

(δ_{H}, 300 MHz, CDCl₃) 1.65 (m, 6H), 2.02 (s, 3H), 3.57 (m, 4H), 4.52 (t, 1H), 4.61 (t, 1H), 4.96 (dd, 1H), 5.12 (dd, 1H), 5.89 (m, 1H), 7.55 (t, 1H), 7.70 (d, 1H), 7.77 (m, 1H), 8.31 (d, 1H), 8.45 (m, 1H), 8.55 (s, 1H); ESL 472.1 [M+H].

### 3-Cyano-N-(6-(piperidine-1-carbonyl)-3-(pyridin-3-ylmethyl)-3H-imidazo[4,5-b]pyridin-2-yl)benzamide

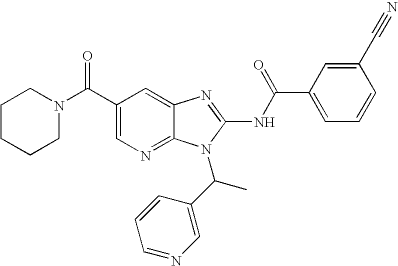

(δ_{H}, 300 MHz, CDCl₃) 1.64 (m, 6H), 2.17 (d, 3H), 3.56 (m, 4H), 6.59 (q, 1H), 7.40 (d, 1H), 7.55 (t, 1H), 7.64 (d, 1H), 7.75 (m, 1H), 8.27 (d, 1H), 8.42 (m, 1H), 8.60 (s, 1H), 8.55 (m, 3H); ESI, 480.2 [M+H].

### 3-Cyano-N-[6-[4-(2-hydroxy-ethyl)-piperidine-1-carbonyl]-3-(3-methoxy-propyl)-3H-imidazo[4,5-b]pyridin-2-yl]-benzamide

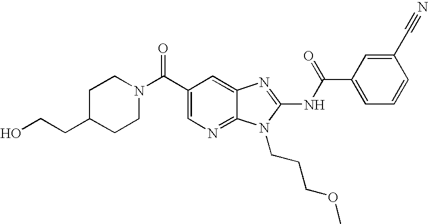

(δ_{H}, 300 MHz, CDCl₃) 1.22 (m, 2H), 1.59 (d, 2H), 1.77 (m, 3H), 2.20 (t, 3H), 2.98 (m, 2H), 3.30 (s, 3H), 3.49 (t, 2H), 3.82 (m, 3H), 4.99 (t, 2H), 4.69 (m, 1H), 7.56 (t, 1H), 7.67 (s, 1H), 7.78 (d, 1H), 8.33 (s, 1H), 8.51 (d, 1H), 8.65 (s, 1H); ESI, 491.2 [M+H].

### N-Butyl-2-(3-cyanobenzamido)-3-(3-methoxypropyl)-N-methyl-3H-imidazo[4,5-b]pyridine-6-carboxamide

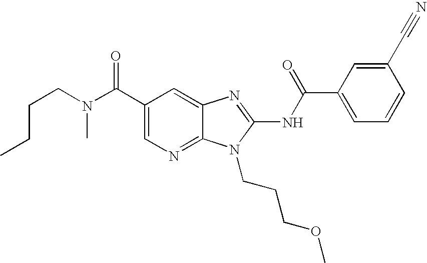

(δ_{H}, 300, CDCl₃) 0.84 (m, 3H), 1.14 (m, 1H), 1.33 (m, 1H), 1.54 (m, 2H), 2.16 (m, 2H), 3.01 (m, 3H), 3.25 (m, 4H), 3.44 (m, 3H), 4.43 (t, 2H), 7.50 (t, 1H), 7.61 (s, 1H), 7.71 (d, 1H), 8.29 (s, 1H), 8.49 (d, 1H), 8.62 (s, 1H); ESI, 449.2 [M+H].

### 3-Cyano-N-(3-(2-(methylamino)ethyl)-6-(piperidine-1-carbonyl)-3H-imidazo[4,5-b]pyridin-2-yl)benzamide

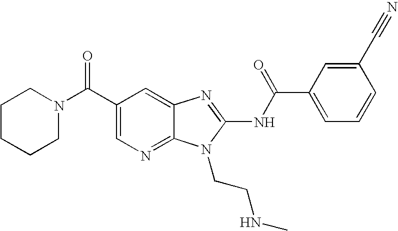

(δ_{H}, 300 MHz, CD₃OD) 1.74 (m, 6H), 2.80 (s, 3H), 3.48 (s, 2H), 3.70 (m, 4H), 4.77 (t, 2H), 7.66 (m, 1H), 7.82 (d, 1H), 7.89 (m, 1H), 8.33 (d, 1H), 8.56 (m, 1H), 8.64 (m, 1H); ESI, 432.1 [M+H].

### 3-Cyano-N-(6-(piperidine-1-carbonyl)-3-(pyrimidin-4-ylmethyl)-3H-imidazo[4,5-b]pyridin-2-yl)benzamide

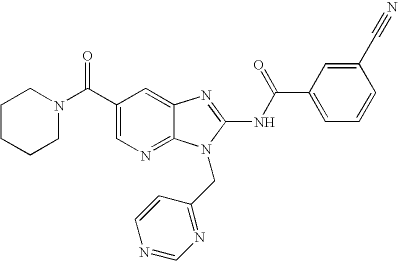

δ_{H}, 300 MHz, CDCl₃) 1.69 (m, 6H), 3.60 (m, 4H), 5.66 (s, 2H), 7.33 (dd, 1H), 7.54 (t, 2H), 7.75 (m, 2H), 8.33 (d, 1H), 8.42 (m, 1H), 8.51 (m, 1H), 8.73 (d, 1H), 9.14 (d, 1H); ESI, 467.2 [M+H].

### 3-Cyano-N-(6-(piperidine-1-carbonyl)-3-((tetrahydrofuran-3-yl)methyl)-3H-imidazo[4,5-b]pyridin-2-yl)benzamide

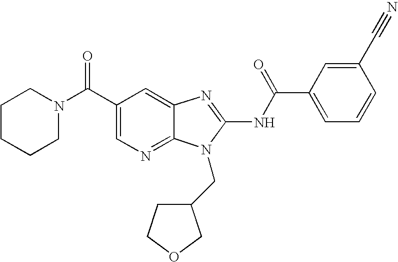

(δ_{H}, 300 MHz, CDCl₃) 1.61 (m, 6H), 1.83 (m, 1H), 2.00 (m, 1H), 3.04 (m, 1H), 3.53 (m, 4H), 3.78 (m, 3H), 3.99 (m, 1H), 4.35 (m, 2H), 7.53 (t, 1H), 7.65 (d, 1H), 7.73 (d, 1H), 8.30 (d, 1H), 8.48 (d, 1H), 8.57 (s, 1H); ESI, 459.1 [M+H].

### Methyl 2-(3-fluorobenzamido)-3-(3-methoxypropyl)-3H-imidazo[4,5b]pyridine-6-carboxylate

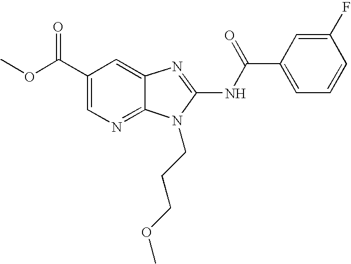

(δ_{H}, 300 MHz, CDCl₃) 2.21 (m, 2H), 3.26 (s, 3H), 3.48 (t, 2H, 3.94 (s, 3H), 4.48 (t, 2H), 7.18 (m, 1H), 7.40 (m, 1H), 8.02 (m, 1H), 8.10 (m, 2H), 8.94 (d, 1H); ESI, 387.2 [M+H].

### 3-Cyano-N-[6-(2,5-dimethyl-pyrrolidine-1-carbonyl)-3-(3-methoxy-propyl)-3H-imidazo[4,5-b]pyridin-2-yl]-benzamide

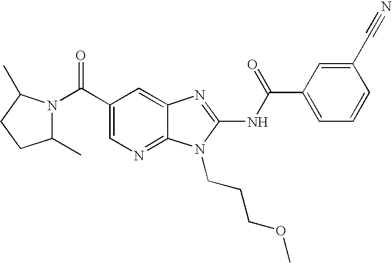

(δ_{H}, 300 MHz, CDCl₃) 1.30 (bs, 5H), 1.75 (bs, 3H), 2.19 (m, 4H), 3.31 (s, 3H), 3.49 (s, 2H), 4.20 (bs, 2H), 4.50 (t, 2H), 7.58 (t, 1H), 7.68 (s, 1H), 7.78 (d, 1H), 8.42 (s, 1H), 8.54 (d, 1H), 8.71 (s, 1H); ESI, 461.2 [M+H].

### (R)-3-Cyano-N-[6-(3-dimethylamino-pyrrolidine-1-carbonyl)-3-(3-methoxy propyl)-3H-imidazo[4,5-b]pyridin-2-yl]-benzamide

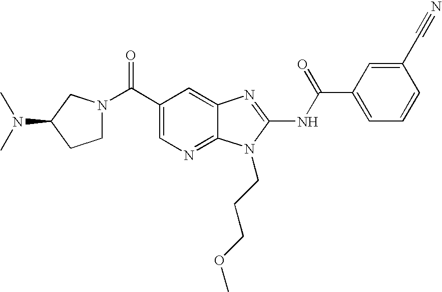

(δ_{H}, 300 MHz, CDCl₃) 2.16 (m, 2H), 2.43 (m, 2H), 2.92 (s, 6H), 3.27 (s, 3H), 3.46 (t, 2H), 3.77 (m, 3H), 3.91 (m, 2H), 4.42 (t, 2H), 7.50 (t, 1H), 7.71 (d, 2H), 7.87 (s, 1H), 8.48 (m, 3H); ESI, 476.1 [M+H].

### 2-(3-Cyano-benzoylamino)-3-(3-methoxy-propyl)-3H-imidazo[4,5-b]pyridine-6-carboxylic acid butylamide

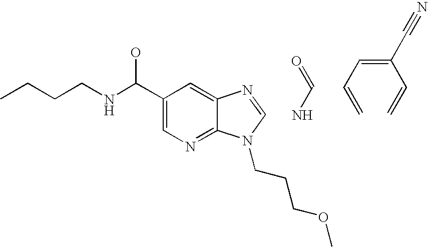

(δ_{H}, 300 MHz, CDCl₃) 0.98 (t, 3H), 1.45 (m, 2H), 1.65 (m, 2H), 2.23 (m, 2H), 3.40 (s, 3H), 3.52 (m 4H), 4.51 (t, 2H), 6.35 (bt, 1H), 7.59 (t, 1H), 7.80 (m, 1H), 8.03 (d, 1H), 8.55 (m, 1H), 8.64 (d, 1H), 8.68 (m, 1H); m/z ESI 435 [M+H].

### 2-(3-Cyanobenzamido)-3-(3-methoxypropyl)-N-(pyridin-2-ylmethyl)-3H-imidazo[4,5-b]pyridine-6-carboxamide

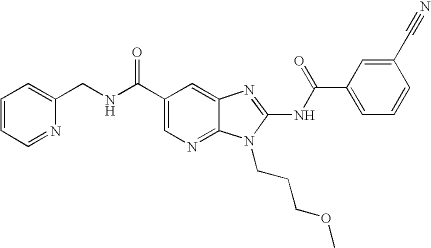

(δ_{H}, 300 MHz, CDCl₃) 2.22 (m, 2H), 3.28 (s, 3H), 3.49 (t, 2H), 4.49 (t, 2H), 4.79 (m, 2H), 7.32 (m, 1H), 7.58 (t, 1H), 7.73 (m, 3H), 8.10 (m, 1H), 8.55 (m, 2H), 8.67 (s, 1H), 8.81 (m, 1H); ESI, 470.3 [M+H].

### 3-Cyano-N-(6-(piperidine-1-carbonyl)-3-(tetrahydro-2H-pyran-3-yl)-3H-imidazo[4,5-b]pyridin-2-yl)benzamide

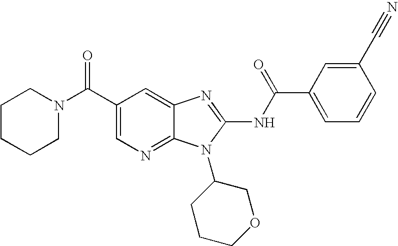

(δ_{H}, 300 MHz, CDCl₃) 1.60 (m, 6H), 1.95 (m, 3H), 2.91 (m, 1H), 3.54 (m, 5H), 3.98 (m, 2H), 4.42 (t, 1H), 5.11 (m, 1H), 7.53 (t, 1H), 7.62 (m, 1H), 7.74 (d, 1H), 8.27 (m, 1H), 8.50 (d, 1H), 8.57 (s, 1H); ESI, 459.1 [M+H].

### 3-Cyano-N-(6-(piperidine-1-carbonyl)-3-(tetrahydrofuran-3-yl)-3H-imidazo[4,5-b]pyridin-2-yl)benzamide

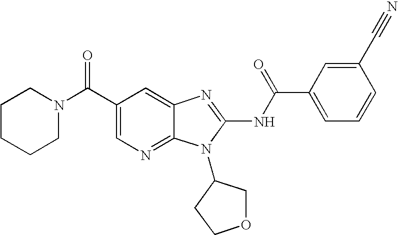

(δ_{H}, 300 MHz, CDCl₃) 1.70 (m, 6H), 2.45 (m, 1H), 2.78 (m, 1H), 3.51 (m, 4H), 4.10 (m, 1H), 4.20 (t, 1H), 4.32 (dd, 1H), 4.47 (m, 1H), 5.73 (m, 1H), 7.56 (t, 1H), 7.65 (d, 1H), 7.76 (m, 1H), 8.33 (d, 1H), 8.52 (m, 1H), 8.61 (s, 1H); ESI, 445.1 [M+H].

### 2-(3-Cyano-benzoylamino)-3-(3-methoxy-propyl)-3H-imidazo[4,5-b]pyridine-6-carboxylic acid ethyl ester

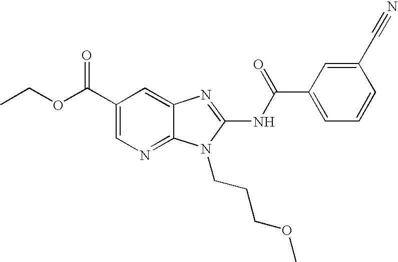

(δ_{H}, 300 MHz, CDCl₃) 1.43 (t, 3H), 2.22 (m, 2H), 3.30 (s, 3H), 3.50 (t, 2H), 4.45 (q, 2H), 4.52 (t, 2H), 7.59 (m, 1H), 7.80 (dd, 1H), 8.18 (d, 1H), 8.56 (dd, 1H), 8.68 (s, 1H), 9.00 (d, 1H); ESI, 408 [M+H].

### 2-(3-Cyanobenzamido)-N-cyclohexyl-3-(3-methoxypropyl)-N-methyl-3H-imidazo[4,5-b]pyridine-6-carboxamide

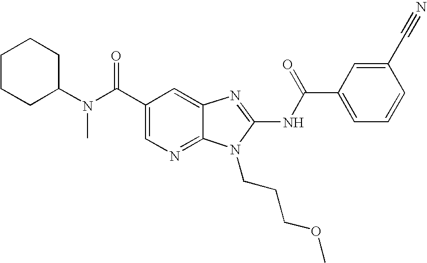

(δ_{H}, 300 MHz, CDCl₃) 1.08 (m, 2H), 1.35-1.90 (m, 9H), 2.21 (m, 2H), 2.80-3.05 (m, 3H), 3.29 (s, 3H), 3.50 (m, 2H), 4.48 (m, 2H), 7.56 (t, 1H), 7.63 (s, 1H), 7.76 (d, 1H), 8.32 (s, 1H), 8.53 (d, 1H), 8.66 (s, 1H); ESI, 475 [M+H].

### 3-Cyano-N-(3-(3-methoxypropyl)-5-(piperidine-1-carbonyl)-3H-imidazo[4,5-b]pyridin-2-yl)benzamide

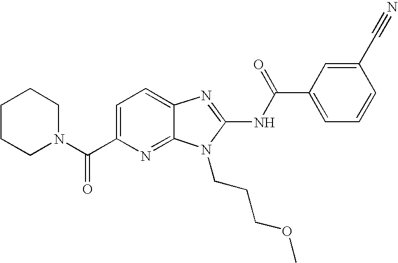

(δ_{H}, 300 Hz, CDCl₃) 1.70 (m, 6H), 2.19 (m, 2H), 3.27 (s, 3H), 3.47 (t, 2H), 3.52 (m, 2H), 3.77 (m, 2H), 4.46 (t, 2H), 7.56 (m, 2H), 7.65 (d, 1H), 7.77 (d, 1H), 8.51 (d, 1H), 8.65 (s, 1H); ESI, 447 [M+H].

### 3-Cyano-N-(3-pentyl-6-(piperidine-1-carbonyl)-3H-imidazo[4,5-b]pyridin-2-yl)benzamide

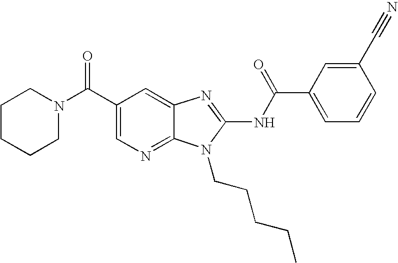

(δ_{H}, 300 Hz, CDCl₃) 0.90 (t, 3H), 1.40 (m, 4H), 1.65 (m, 6H), 1.94 (m, 2H), 3.54 (m, 4H), 4.34 (t, 2H), 7.55 (t, 1H), 7.63 (d, 1H), 7.76 (m, 1H), 8.33 (d, 1H), 8.51 (m, 1H), 8.62 (t, 1H); ESL 445.2 [M+H].

### 3-Cyano-N-[6-(3-hydroxy-piperidine-1-carbonyl)-3-(3-methoxy-propyl)-3H-imidazo[4,5-b]pyridin-2-yl]-benzamide

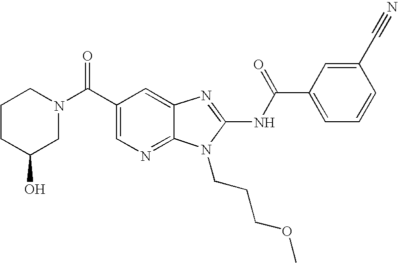

(δ_{H}, 300 MHz, CDCl₃) 1.50-2.10 (m, 4H), 2.22 (m, 2H), 3.31 (s, 3H), 3.53 (m, 4H), 3.80-4.10 (m, 3H), 4.52 (t, 2H), 7.59 (m, 2H), 7.80 (m, 1H), 8.46 (m, 2H), 8.58 (s, 1H); ESI, 463 [M+H].

### (R)-3-Fluoro-N-(3-(3-methoxypropyl)-6-(2-methylpyrrolidine-1-carbonyl)-3H-imidazo[4,5-b]pyridin-2-yl)benzamide

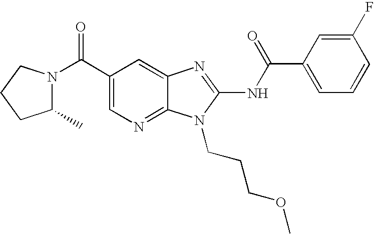

(δ_{H}, 300 MHz, CDCl₃) 1.28 (d, 3H), 1.70 (m, 5H), 2.17 (m, 2H), 3.25 (s, 3H), 3.44 (t, 2H), 3.80 (m, 2H), 4.43 (t, 2H), 7.15 (m, 1H), 7.36 (m, 1H), 7.61 (d, 1H), 7.64 (s, 1H), 7.99 (d, 1H), 8.10 (d, 1H), 8.41 (s, 1H); ESI, 440.2 [M+H].

### 3-Cyano-N-(6-(2-ethylpyrrolidine-1-carbonyl)-3-(3-methoxypropyl)-3H-imidazo[4,5-b]pyridin-2-yl)benzamide

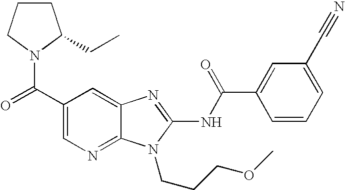

(δ_{H}, 300 MHz, CDCl₃) 0.65-0.95 (m, 3H), 1.20-1.80 (m, 3H), 1.90-2.25 (m, 5H), 3.29 (s, 3H), 3.48 (m, 4H), 4.23 (m, 1H), 4.48 (t, 2H), 7.56 (t, 1H), 7.77 (m, 2H), 8.51 (m, 2H), 8.66 (s, 1H); ESL 461 [M+H].

### 3-Cyano-N-[3-(3-methoxy-propyl)-6-(3-methoxy-pyrrolidine-1-carbonyl)-3H-imidazo[4,5-b]pyridin-2-yl]-benzamide

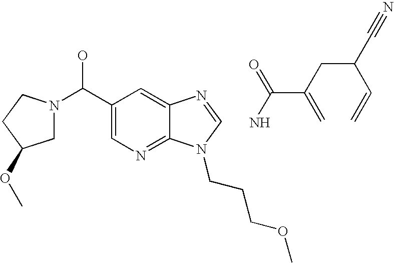

(δ_{H}, 300 MHz, CDCl₃) 1.96 (m, 1H), 2.19 (m, 3H), 3.31 (d, 6H), 3.51 (m, 3H), 3.77 (m, 3H), 3.91 (d, 1H), 4.48 (t, 2H), 7.55 (t, 1H), 7.76 (d, 1H), 7.86 (s, 1H), 8.49 (t, 2H), 8.60 (s, 1H); ESI, 463.2 [M+H].

### (R)-3-Fluoro-N-(3-(3-methoxypropyl)-5-(2-methylpiperidine-1-carbonyl)-3H-imidazo[4,5-b]pyridin-2-yl)benzamide

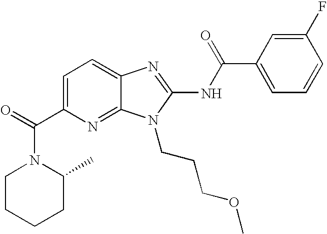

(δ_{H}, 300 MHz, CDCl₃) 1.34 (d, 3H), 1.7 (m, 7H), 2.18 (m, 2H), 3.25 (s, 3H), 3.46 (t, 2H), 4.03 (m, 2H), 4.45 (t, 2H), 7.20 (m, 1H), 7.41 (m, 1H), 7.51 (d, 1H), 7.64 (d, 1H), 7.99 (m, 1H), 8.08 (d, 1H); ESI, 454.2 [M+H].

### 2-(3-Fluorobenzamido)-N-isopropyl-3-(3-methoxypropyl)-N-methyl-3H-imidazo[4,5-b]pyridine-6-carboxamide

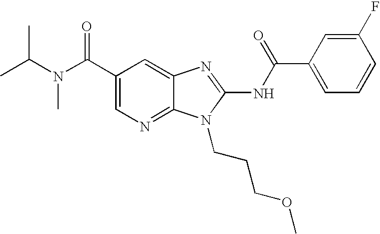

(δ_{H}, 300 MHz, CDCl₃) 1.13 (d, 6H), 2.17 (m, 2H), 2.83 (m, 4H), 3.26 (s, 3H), 3.45 (t, 2H), 4.43 (t, 2H), 7.15 (m, 1H), 7.36 (m, 1H), 7.53 (d, 1H), 7.99 (d, 1H), 8.07 (d, 1H), 8.27 (d, 1H); ESI, 428.2 [M+H].

### 3-Cyano-N-(3-(3-ethoxypropyl)-6-(morpholine-4-carbonyl)-3H-imidazo[4,5-b]pyridin-2-yl)benzamide

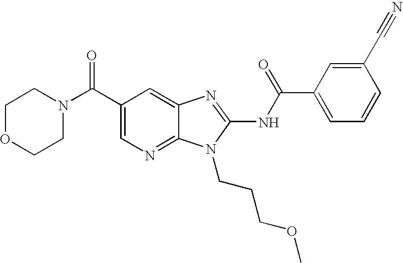

(δ_{H}, 300 MHz, CDCl₃) 1.10 (t, 3H), 2.21 (m, 2H), 3.38 (q, 2H), 3.51 (t, 2H), 3.73 (bs, 8H), 4.49 (t, 2H), 7.56 (t, 1H), 7.67 (d, 1H), 7.77 (m, 1H), 8.35 (d, 1H), 8.52 (m, 1H), 8.63 (t, 1H); ESL 463.2 [M+H].

### 2-(3-Fluorobenzamido)-N-isopropyl-3-(3-methoxypropyl)-N-methyl-3H-imidazo[4,5-b]pyridine-5-carboxamide

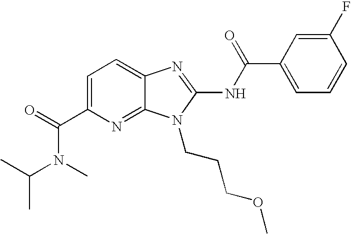

(δ_{H}, 300 MHz, CDCl₃) 1.24 (d, 6H), 2.18 (m, 2H), 2.96 (d, 3H), 3.26 (s, 3H), 3.46 (t, 2H), 4.19 (m, 1H), 4.43 (m, 2H), 7.19 (m, 1H), 7.40 (m, 1H), 7.54 (t, 1H), 7.61 (d, 1H), 8.01 (m, 1H), 8.09 (d, 1H); ESI, 428.1 [M+H].

### N-(3-(4-Fluorobenzyl)-6-(piperidine-1-carbonyl)-3H-imidazo[4,5-b]pyridin-2-yl)-3-cyanobenzamide

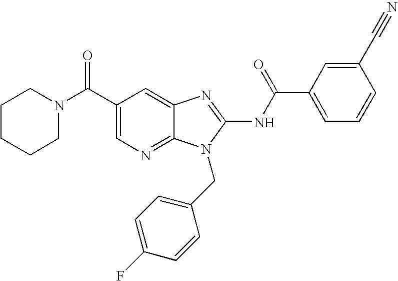

(δ_{H}, 300 MHz, CDCl₃) 1.64 (m, 6H), 3.58 (m, 4H), 5.50 (s, 2H), 7.00 (t, 2H), 7.57 (m, 3H), 7.70 (d, 10H), 7.79 (d, 1H), 8.37 (d, 1H), 8.49 (d, 1H), 8.60 (s, 1H); ESI, 483.1 [M+H].

### 3-Cyano-N-[6-(3-hydroxymethyl-piperidine-1-carbonyl)-3-(3-methoxy-propyl)-3H-imidazo[4,5-b]pyridin-2-yl]-benzamide

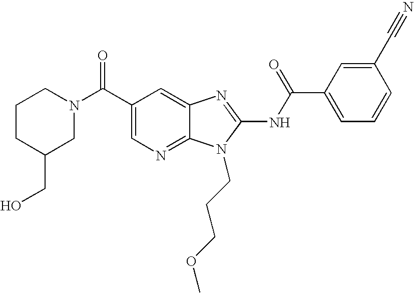

(δ_{H}, 300 MHz, CDCl₃) 1.70 (m, 5H), 2.21 (s, 2H), 3.01 (s, 1H), 3.30 (s, 3H), 3.54 (m, 5H), 4.08 (m, 2H), 4.49 (s, 2H), 7.57 (s, 1H), 7.75 (m, 2H), 8.36 (m, 1H), 8.52 (s, 1H), 8.65 (s, 1H); ESI, 477.2 [M+H].

Although the foregoing invention has been described in some detail for purposes of illustration, it will be readily apparent to one skilled in the art that changes and modifications may be made without departing from the scope of the invention described herein.

We claim:

1. A compound of formula 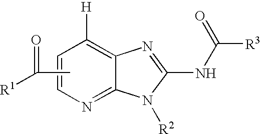
wherein
R¹ is selected from the group consisting of OR⁴, NR⁵R⁶, heterocyclyl and substituted heterocyclyl;
R² is selected from the group consisting of
(a) C₁-C₂₀ hydrocarbon;
(b) C₃-C₂₀ hydrocarbon in which
(i) from one to three —CH₂— are replaced by —O—, —S(O)ₘ—, —NH— or —(C═O)—, wherein m is 0, 1 or 2; or
(ii) one 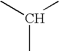
is replaced by 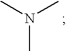
(c) heteroaryl and
(d) heteroarylalkyl;
R³ is selected from the group consisting of aryl, arylalkyl, heteroaryl, heteroarylalkyl, substituted aryl, substituted arylalkyl, substituted heteroaryl and substituted heteroarylalkyl;
R⁴ is selected from the group consisting of H, alkyl, aryl, arylalkyl, heteroaryl, heteroarylalkyl, substituted aryl, substituted arylalkyl, substituted heteroaryl and substituted heteroarylalkyl;
R⁵ is selected from the group consisting of H, C₁-C₂₀ hydrocarbon, heterocyclyl, heterocyclylalkyl, substituted alkyl, oxaalkyl, substituted aryl, substituted arylalkyl, substituted heterocyclyl and substituted heterocyclylalkyl; and
R⁶ is selected from the group consisting of H, (C₁-C₆)alkyl, (C₁-C₆)alkoxy, aryl and substituted (C₁-C₆)alkyl.

2. A compound according to claim 1 of formula II:

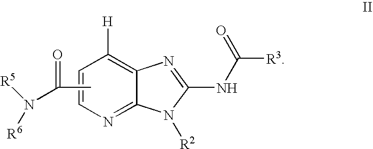

3. A compound according to claim 1 of formula III:

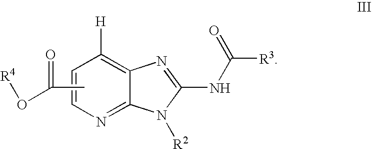

4. A compound of formula IV according to claim 1 wherein R¹ is an optionally substituted nitrogen-attached heterocycle:

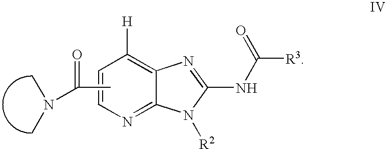

5. A compound according to claim 2 wherein R⁵ is C₁-C₂₀ hydrocarbon.

6. A compound according to claim 2 wherein R⁶ is hydrogen or (C₁-C₃)alkyl and R⁵ is —(CH₂)ₙ-cyc, wherein n is 1 to 4 and cyc is carbocyclyl or heterocyclyl, said carbocyclyl or heterocyclyl optionally substituted with from one to three halogen, (C₁-C₃)alkyl, hydroxy or (C₁-C₃)alkoxy.

7. A compound according to claim 6 wherein said cyc is chosen from optionally substituted phenyl, pyridinyl, imidazolyl and pyrrolidinyl.

8. A compound according to claim 4 wherein said nitrogen attached heterocycle is a four- or seven-membered heterocycle.

9. A compound according to claim 4 wherein said nitrogen attached heterocycle is chosen from morpholine, thiomorpholine and piperazine.

10. A compound according to claim 4 wherein said nitrogen attached heterocycle is chosen from 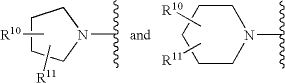
wherein R¹⁰ and R¹¹ are independently chosen from H, (C₁-C₃)alkyl, halogen, halo(C₁-C₃)alkyl, hydroxy, hydroxy(C₁-C₃)alkyl, (C₁-C₃)oxaalkyl and (C₁-C₃)alkoxy, or
taken together R¹⁰ and R¹¹ form a fused six-membered ring optionally substituted with (C₁-C₃)alkyl, halogen, halo(C₁-C₃)alkyl, hydroxy or (C₁-C₃)alkoxy.

11. A compound according to claim 1 wherein R¹ is optionally substituted aryl or heteroaryl.

12. A compound according to claim 1 wherein R³ is: 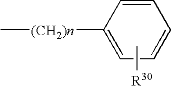
where n is 0 or an integer selected from 1-4; and
R³⁰ is selected from H, halogen, cyano, nitro, formyl alkoxy, alkyl, haloalkyl, alkynyl and heteroaryl.

13. A compound according to claim 12 wherein R³⁰ is meta fluoro or meta cyano.

14. A compound according to claim 1 wherein R³ is heteroaryl or substituted heteroaryl.

15. A compound according to claim 1 wherein R² is chosen from methoxyphenyl, 1-acetylpiperidinyl, 1-acetylpyrrolidinyl, 1-acetylazetidinyl tetrahydrofuranyl, tetrahydrofuranylmethyl, tetrahydropyranyl, pyridinylmethyl, (imidazolyl)ethyl, methylpiperidinyl, pyrimidinylmethyl, (acetylamino)(C₁-C₆)alkyl, hydroxy(C₁-C₆)alkyl, methylthio(C₁-C₆)alkyl, α-pyridinylethyl, cyclopropyl, [(C₁-C₆)alkoxycarbonyl](C₁-C₆)alkyl, (methylamino)(C₁-C₆)alkyl, α(methoxyphenyl)ethyl and (dimethoxyphenyl)methyl.

16. A compound according to claim 1 wherein R² is oxaalkyl.

17. A pharmaceutical composition comprising a pharmaceutically acceptable carrier and a therapeutically effective amount of at least one compound according to claim 1.

18. A method of treating a disorder which is mediated by adenosine receptor function, which comprises administering to a subject in need of such treatment a therapeutically effective amount of a compound according to claim 1.

19. A method according to claim 18 wherein the disorder is selected from the group consisting of central nervous system (CNS) and peripheral nervous system (PNS) diseases; neurodegenerative diseases; cardiovascular diseases; cognitive disorders; CNS injury; renal ischemia; acute and chronic pain; affective disorders; cognitive disorders; central nervous system injury; cerebral ischemia; myocardial ischemia; muscle ischemia; sleep disorders; eye disorders, restless leg syndrome and diabetic neuropathy.

20. A method according to claim 19 wherein the CNS and PNS disorders are movement disorders.

21. A method according to claim 20 wherein the movement disorder is selected from the group consisting of a disorder of the basal ganglia which results in dyskinesias Huntington's disease, multiple system atrophy, progressive supernuclear palsy, essential tremor, myoclonus, corticobasal degeneration, Wilson's disease, progressive pallidal atrophy, Dopa-responsive dystoma-Parkinsonism, spasticity, Alzheimer's disease and Parkinson's disease.

22. A method according to claim 20 wherein the movement disorder is Parkinson's disease.

23. A method according to claim 19 for treating restless leg syndrome.
